(19) United States
(12) Patent Application Publication
(10) Pub. No.: US 2025/0207144 A1
(43) Pub. Date: Jun. 26, 2025

(54) ENDOGENOUS PLANT EXPRESSION ENHANCER

(71) Applicant: Aramis Biotechnologies Inc., Quebec (CA)

(72) Inventors: Pierre-Olivier Lavoie, Quebec (CA); Marc-Andre D'Aoust, Quebec (CA); Ko Kato, Ikoma-shi (JP); Shotaro Yamasaki, Ikoma-shi (JP)

(73) Assignee: Aramis Biotechnologies Inc., Quebec (CA)

(21) Appl. No.: 18/912,580

(22) Filed: Oct. 10, 2024

Related U.S. Application Data
(62) Division of application No. 17/439,155, filed on Sep. 14, 2021, now Pat. No. 12,146,145.

(51) Int. Cl. C12N 15/82

(52) U.S. Cl. CPC C12N 15/8216; C12N 15/8258

(57) ABSTRACT

An isolated expression enhancer active in a plant, portion of a plant or plant cell, the expression enhancer is provided. The isolated expression enhancer may be selected from the group consisting of nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5). Methods for using the isolated expression enhancer are also provided.

### CROSS-REFERENCE TO RELATED APPLICATIONS

The present application is a continuation of U.S. application Ser. No. 17/439,155 filed Sep. 14, 2021, which is a national phase filing under 35 U.S.C. § 371 of International Application No. PCT/CA2019/050319, filed on Mar. 14, 2019, the contents of which is incorporated herein by reference in its entirety.

### INCORPORATION OF THE SEQUENCE LISTING

The contents of the electronic sequence listing (186360018U2 2025_02_24.xml; Size: 121,896 bytes; and Date of Creation: Feb. 24, 2025) is herein incorporated by reference in its entirety.

### FIELD OF INVENTION

The present invention relates to expression enhancers that are active in plants. The present invention also relates to the expression of proteins of interest in plants and provides methods and compositions for production of proteins of interest in plants.

### BACKGROUND OF THE INVENTION

Plants offer great potential as production systems for recombinant proteins. One approach to producing foreign proteins in plants is to generate stable transgenic plant lines. However this is a time consuming and labor intensive process. An alternative to transgenic plants is the use of plant virus-based expression vectors. Plant virus-based vectors allow for the rapid, high level, transient expression of proteins in plants.

High level transient expression of foreign proteins in plants has been obtained using of vectors based on RNA plant viruses, including comoviruses, such as Cowpea mosaic virus (CPMV; see, for example, WO2007/135480; WO2009/087391; US 2010/0287670, Sainsbury F. et al., 2008, Plant Physiology; 148: 121-1218: Sainsbury F. et al., 2008, Plant Biotechnology Journal; 6: 82-92; Sainsbury F. et al., 2009, Plant Biotechnology Journal; 7: 682-693; Sainsbury F. et al. 2009, Methods in Molecular Biology. Recombinant Proteins From Plants, vol. 483:25-39). Modifications of the 5′UTR of the RNA-2 of the comovirus cowpea mosaic virus (CPMV) have resulted in additional expression enhancer activity (as determined level of expression of a nucleic acid of interest or a protein of interest), when compared to the wild type CPMV 5′UTR. For example, mutation of the start codon at position 161 in a CPMV RNA-2 vector (U162C; HT) increases the levels of expression of a protein encoded by a sequence inserted after the start codon at position 512. This permits the production of high levels of foreign proteins without the need for viral replication and was termed the CPMV-HT system (WO2009/087391; Sainsbury and Lomonossoff, 2008, Plant Physiol. 148, 1212-1218). In pEAQ expression plasmids (Sainsbury et al., 2009, Plant Biotechnology Journal, 7, pp 682-693; US 2010/0287670), the sequence to be expressed is positioned between the 5′UTR and the 3′ UTR. The 5′UTR in the pEAQ series carries the U162C (HT) mutation.

Additional modification of the CPMV 5′ UTR region, have been described that further increase expression of a nucleic acid of interest within a plant. For example, “CMPV HT+” (comprising nucleotides 1-160 of the CPMV 5′ UTR with modified ATGs at position 115-117, and at position 161-163; WO2015/143567; which is incorporated herein by reference), and “CPMVX” (where X=160, 155, 150, or 114 nucleic acids in length; WO 2015/103704; which is incorporated herein by reference). An example of CMPVX is the expression enhancer “CPMV 160”. Expression of a nucleic acid sequence operatively linked to CPMV HT+” resulted in a significant increase in production of a protein of interest that was encoded by the nucleic acid sequence, when compared to the production of the same protein of interest using the same nucleic acid sequence operatively linked to the “CPMV HT” expression enhancer (see FIGS. 2 and 3 of WO2015/143567). Furthermore, expression of a nucleic acid sequence operatively linked to the “CPMV 160” expression enhancer resulted in a significant increase in production of a protein of interest encoded by the nucleic acid sequence, when compared to the production of the same protein of interest using the same nucleic acid sequence operatively linked to the “CPMV HT+” expression enhancer (see FIGS. 2 and 3 of WO2015/143567).

Diamos et.al (Frontiers in Plant Science. 2016, vol 7 pp. 1-15; which is incorporated herein by reference) describe several expression enhancers that may be used to increase production of proteins in plants (see Table 2 of Diamos et. al.), including the expression enhancer NbPsaK2 3′. As shown in FIG. 4 of Diamos et.al. (2016), production of protein of interest encoded by a nucleci acid that was operatively linked to NbPsaK2 3′ resulted in enhanced protein production when compared to the production of the same protein encoded by the same nucleic acid sequence operatively linked to other truncated psaK expression enhancers.

### SUMMARY OF THE INVENTION

The present invention relates to expression enhancers that are active in plants. The present invention also relates to the expression of proteins of interest in plants, and provides methods and compositions for the production of proteins of interest in plants.

It is an object of the invention to provide an improved expression enhancer active in a plant.

According to the present invention there is provided an isolated expression enhancer active in a plant, the expression enhancer selected from the group consisting of:

```
  (SEQ ID NO: 1)
nbEPI42;
  (SEQ ID NO: 2)
nbSNS46;
  (SEQ ID NO: 3)
nbCSY65;
  (SEQ ID NO: 4)
nbHEL40;
  (SEQ ID NO: 5)
nbSEP44;
```

and a nucleic acid having from 90-100% sequence identity to the nucleotide sequence set forth in any one of SEQ ID NO's:1-5. Wherein, the expression enhancer, when operatively linked to a nucleic acid of interest, for example a heterologous nucleic acid of interest, results in expression of the nucleic acid of interest. Additionally, the expression enhancer, when operatively linked to a nucleic acid of interest, for example a heterologous nucleic acid of interest, may increase the level of expression of the nucleic acid of interest, or the heterologous nucleic acid of interest, when compared to the level of expression of the same nucleic acid or heterologous nucleic acid of interest that is not operatively linked to the expression enhancer, or for example, when operatively linked to the prior art expression enhancer CPMV 160 (SEQ ID NO:6).

The present disclosure also provides for a nucleic acid sequence comprising one of the isolated expression enhancers as described above, the expression enhancer operatively linked with a heterologous nucleotide sequence encoding a protein of interest. The heterologous nucleotide sequence may encode a viral protein or an antibody, for example which is not to be considered limiting, the viral protein may be an influenza protein or a norovirus protein. If the protein of interest is an influenza protein then it may include M2, a hemagglutinin protein selected from the group of H1, H2, H3, H4, H5, H6, H7, H8, H9, H10, H11, H12, H13, H14, H15, H16, an influenza type B hemagglutinin, or a combination thereof. If the protein of interest is a norovirus protein, then it may include a VP1 protein, a VP2 protein, or a combination thereof, selected from the group of GI.1, GI.2, GI.3, GI.5, GI.7, GII.1, GII.2, GII.3, GII.4, GII.5, GII.6, GII.7, GII.12, GII.13, GII.14, GII.17 and GII.21.

The present invention also provides a plant expression system comprising one or more than one of the nucleic acid sequence described above. The plant expression system may further comprise a comovirus 3′ UTR.

The present invention also provides a plant expression system comprising one or more than one of the isolated nucleic acid sequence operatively linked with a heterologous nucleic acid, or nucleotide sequence, as described above. The plant expression system may further comprise a comovirus 3′ UTR.

Also disclosed herein is a method of producing a protein of interest in a plant or in a portion of a plant comprising, introducing into the plant or in the portion of a plant the plant expression system as described above, comprising the one or more than one of nucleic acid sequence, and incubating the plant, the portion of a plant, or plant cell, under conditions that permit expression of each of the heterologous nucleotide sequence encoding the protein of interest. For example, the protein of interest may be a viral protein, such as an influenza protein or a norovirus protein. If the protein of interest is an influenza protein then it may include M2, a hemagglutinin protein selected from the group of H1, H2, H3, H4, H5, H6, H7, H8, H9, H10, H11, H12, H13, H14, H15, H16, an influenza type B hemagglutinin, and a combination thereof. If the protein of interest is a norovirus protein, then it may include a VP1 protein, a VP2 protein, or a combination thereof, selected from the group of GI.1, GI.2, GI.3, GI.5, GII.1, GII.2, GII.3, GII.4, GII.5, GII.6, GII.7, GII.12, GII.13, GII.14, GII.17 and GII.21.

A method of producing a multimeric protein of interest, is also described herein. The method involves co-expressing two or more than two of the nucleic acid sequence as described above, in a plant, the portion of a plant, or plant cell, in a transient or stable manner, wherein each of the two or more than two of the nucleic acid sequence encodes a component of the multimeric protein, and incubating the plant, the portion of a plant, or plant cell, under conditions that permit expression of each of the heterologous nucleotide sequence encoding the multimeric protein of interest.

Also provided herein is a plant, a portion of a plant, or plant cell that is transiently transformed, or stably transformed, with plant expression system as described above.

A plant-based expression system comprising an expression enhancer as described herein results in expression of the nucleic acid of interest. Furthermore, the plant-based expression system comprising an expression enhancer as described herein may result in increasing or enhancing expression of a nucleotide sequence encoding a heterologous open reading frame that is operatively linked to the expression enhancer, as described herein. The increase in expression may be determined by comparing the level of expression obtained using the expression enhancer as described herein with the level of expression of the same nucleotide sequence encoding the heterologous open reading frame but not operatively linked to an expression enhancer, or for example, when operatively linked to the prior art expression enhancer CPMV 160 (SEQ ID NO:6).

The plant based expression systems, vectors, constructs and nucleic acids comprising one or more than one expression enhancer as described herein may also have a number of properties such as, for example, containing convenient cloning sites for genes or nucleotide sequences of interest, they may be used to easily transform plants in a cost-effective manner, they may cause efficient local or systemic transformation of inoculated plants. In addition, the transformation of a plant should provide a good yield of useful protein material.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1A is prior art and shows the relative titer of influenza H1 California, H3 Victoria, H5 Indonesia and B Wisconsin produced in plants by expressing a nucleic acid encoding each of these proteins wherein the nucleic acid is operatively linked to CPMV HT expression enhancer (described in WO 2009/087391) or to a CPMV 160+expression enhancer (described in WO 2015/103704). FIG. 1B is prior art and shows the relative titer of H1 California, H3 Victoria, B Brisbane, B Brisbane+H1Tm, B Massachusetts, B Massachusetts+H1Tm, B Wisconsin and B Wisconsin+H1Tm, produced in plants by expressing a nucleic acid encoding each of these proteins wherein the nucleic acid is operatively linked to a CPMV HT expression enhancer (described in WO 2009/087391) or to a CPMV 160+expression enhancer (described in WO 2015/103704).

FIG. 2A shows relative activity of the Dasher protein (Dasher GFP; FPB-27 269; from ATUM), a cytosolic protein, produced in plants by expressing a nucleic acid encoding the Dasher protein wherein the nucleic acid encoding Dasher is operatively linked to expression enhancers CPMV 160 (SEQ ID NO:6; described in WO 2015/103704), nbMT78 (SEQ ID NO:7; described in U.S. Provisional Application No. 62/643,053 Filed Mar. 14, 2018); atHSP69 (SEQ ID NO:8; described in U.S. Provisional Application No. 62/643,053 Filed Mar. 14, 2018); nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5). FIG. 2B shows relative yield of the cytosolic norovirus GII.4/Sydney 2012 VP1 VLPs, following gradient centrifugation, the VLPs produced in plants by expressing a nucleic acid encoding the norovirus VP1 protein wherein each of the nucleic acid encoding the VP1 protein is operatively linked to: the expression enhancer CPMV 160 (SEQ ID NO:6; described in WO 2015/103704), nbMT78 (SEQ ID NO:7; described in U.S. Provisional Application No. 62/643,053, filed Mar. 14, 2018, which is incorporated herein by reference); atHSP69 (SEQ ID NO:8; described in U.S. Provisional Application No. 62/643,053, filed Mar. 14, 2018, which is incorporated herein by reference); nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5).

FIG. 3A shows the hemagglutinin titers of the secreted VLP of a H3 A Singapore influenza virus, the VLP produced in plants by expressing a nucleic acid encoding the H3 A Singapore protein wherein the nucleic acid encoding this protein is operatively linked to expression enhancers CPMV 160 (SEQ ID NO:6; described in WO 2015/103704), nbMT78 (SEQ ID NO:7; described in U.S. Provisional Application No. 62/643,053, filed Mar. 14, 2018, which is incorporated herein by reference); atHSP69 (SEQ ID NO:8; described in U.S. Provisional Application No. 62/643,053 filed Mar. 14, 2018, which is incorporated herein by reference); nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5). FIG. 3B shows the hemagglutinin titers of the secreted VLP of an H1 A Michigan influenza virus, the VLP produced in plants by expressing a nucleic acid encoding the H1 A Michigan protein wherein the nucleic acid encoding this protein is operatively linked to expression enhancers CPMV 160 (SEQ ID NO:6; described in WO 2015/103704), nbMT78 (SEQ ID NO:7; described in U.S. Provisional Application No. 62/643,053, filed Mar. 14, 2018, which is incorporated herein by reference); atHSP69 (SEQ ID NO:8; described in U.S. Provisional Application No. 62/643,053, filed Mar. 14, 2018, which is incorporated herein by reference); nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5). FIG. 3C shows relative yield of the secreted multimeric protein IgG1 (Rituximab) produced in plants by co-expressing a first nucleic acid encoding the light chain (LC) of the Rituximab antibody, and second nucleic acid encoding the heavy chain (HC) of Rituximab antibody. The sequences encoding the LC and HC were operatively linked to expression enhancers CPMV 160 (SEQ ID NO:6; described in WO 2015/103704), nbMT78 (SEQ ID NO:7; described in U.S. Provisional Application No. 62/643,053, filed March 14, 201, which is incorporated herein by reference)); atHSP69 (SEQ ID NO:8; described in U.S. Provisional Application No. 62/643,053, filed Mar. 14, 2018, which is incorporated herein by reference); nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5).

FIG. 4 shows constructs encoding Dasher: FIG. 4A shows construct 4460 (2X35S-5′ UTR CPMV 160-Dasher (FPB-27-609) CPMV 3′UTR/NOS); FIG. 4B shows construct 4467 (2X35S-5′UTR nbMT78-Dasher (FPB-27-609)-CPMV 3′UTR/NOS); FIG. 4C shows construct 4472 (2X35S-5′UTR atHSP69-Dasher (FPB-27-609)-CPMV 3′UTR/NOS); FIG. 4D shows construct 6380) (2X35S-5′UTR nbEPI42-Dasher (FPB-27-609)-CPMV 3′UTR/NOS); FIG. 4E shows construct 6381 (2X35S-5′UTR nbSNS46-Dasher (FPB-27-609) -CPMV 3′UTR/NOS); FIG. 4F shows construct 6382 (2X35S-5′UTR nbCSY65-Dasher (FPB-27-609)-CPMV 3′UTR/NOS); FIG. 4G shows construct 6383 (2X35S-5′UTR nbHEL40)-Dasher (FPB-27-609)-CPMV 3′UTR/NOS); FIG. 4H shows construct 6384 (2X35S-5 UTR nbSEP44-Dasher (FPB-27-609)-CPMV 3′UTR/NOS).

FIG. 5 shows constructs encoding VP1-GII.4 Sydney 2012: FIG. 5A shows construct 4133 (2X35S-5′UTR CPMV 160-VP1 (GII.4 Syd12)-CPMV 3′UTR/NOS); FIG. 5B shows construct 4163 (2X35S-5′UTR nbMT78-VP1(GII.4 Syd12)-CPMV 3′UTR/NOS); FIG. 5C shows construct 4164 (2X35S-5′UTR atHSP69-VP1 (GII.4 Syd12)-CPMV 3′UTR/NOS); FIG. 5D shows construct 6218 (2X35S-5′UTR atEPI42-VP1 (GII.4 Syd12) -CPMV 3′UTR/NOS); FIG. 5E shows construct 6214 (2X35S-5′UTR atSNS46-VP1 (GII.4 Syd12)-CPMV 3′UTR/NOS); FIG. 5F shows construct 6215 (2X35S-5′UTR atCSY65-VP1 (GII.4 Syd12)-CPMV 3′UTR/NOS); FIG. 5G shows construct 6216 (2X35S-5′UTR atHEL40-VP1 (GII.4 Syd12)-CPMV 3′UTR/NOS); FIG. 5H shows construct 6217 (2X35S-5′UTR atSEP44-VP1 (GII.4 Syd12) -CPMV 3′UTR/NOS).

FIG. 6 shows constructs encoding IgG1 (Rituximab): FIG. 6A shows construct 4641 (2X35S-5′UTR CPMV 160-SpPDI-HC IgG1-CPMV 3′UTR/NOS); FIG. 6B shows construct 4642 (2X35S-5′UTR CPMV 160)-SpPDI-LC IgG1-CPMV 3′UTR/NOS); FIG. 6C shows construct 6600 (2X35S-5′UTR nbMT78-SpPDI-LC IgG1-CPMV 3′UTR/NOS); FIG. 6D shows construct 6601 (2X35S-5′UTR nbMT78-SpPDI-HC IgG1-CPMV 3′UTR/NOS); FIG. 6E shows construct 6602 (2X35S-5′UTR atHSP69-SpPD1-LC IgG1-CPMV 3′UTR/NOS); FIG. 6F shows construct 6603 (2X35S-5′UTR atHSP69-SpPD1-HC IgGI-CPMV 3′UTR/NOS); FIG. 6G shows construct 6604 (2X35S-5′UTR nbEPI42-SpPDI-LC IgG1-CPMV 3′UTR/NOS); FIG. 6H shows construct 6605 (2X35S-5′UTR nbEPI42-SpPDI-HC IgG1-CPMV 3′UTR/NOS); FIG. 6I shows construct 6606 (2X35S-5′UTR nbSNS46-SpPDI-LC IgG1-CPMV 3′UTR/NOS); FIG. 6J shows construct 6607 (2X35S-5′UTR nbSNS46-SpPDI-HC IgG1-CPMV 3′UTR/NOS); FIG. 6K shows construct 6608 (2X35S-5′UTR nbCSY65-SpPDI-LC IgG1-CPMV 3′UTR/NOS); FIG. 6L shows construct 6609 (2X35S-5′UTR nbCSY65-SpPDI-HC IgG1-CPMV 3′UTR/NOS); FIG. 6M shows construct 6610 (2X35S-5′UTR nbHEL40-SpPD1-LC IgG1-CPMV 3′UTR/NOS); FIG. 6N shows construct 6611 (2X35S-5′UTR nbHEL40-SpPDI-HC IgGI-CPMV 3′UTR/NOS); FIG. 6O shows construct 6612 (2X35S-5′UTR nbSEP44-SpPDI-LC IgG1-CPMV 3′UTR/NOS); FIG. 6P shows construct 6613 (2X35S-5′UTR nbSEP44-SpPDI-HC IgG1-CPMV 3′UTR/NOS); Sp: signal peptide.

FIG. 7 shows constructs encoding H1-A/Michigan/45/2015: FIG. 7A shows construct 3703 (2X35S-5′UTR CPMV 160-SpPD1-H1 A-Mich-45-2015-CPMV 3′UTR/NOS); FIG. 7B shows construct 4703 (2X35S-5′UTR nbMT78-SpPD1-H1 A-Mich-45-2015-CPMV 3′UTR/NOS); FIG. 7C shows construct 4704 (2X35S-5′UTR nbHSP69-SpPD1-H1 A-Mich-45-2015-CPMV 3 UTR/NOS); FIG. 7D shows construct 6700 (2X35S-5′UTR nbEPI42-SpPDI-H1 A-Mich-45-2015-CPMV 3′UTR/NOS); FIG. 7E shows construct 6701 (2X35S-5′UTR nbSNS46-SpPD1-H1 A-Mich-45-2015-CPMV 3′UTR/NOS); FIG. 7F shows construct 6702 (2X35S-5′UTR nbCSY65-SpPD1-H1 A-Mich-45-2015-CPMV 3′UTR/NOS); FIG. 7G shows construct 6703 (2X35S-5′UTR nbHEL40-SpPD1-H1 A-Mich-45-2015-CPMV 3′UTR/NOS); FIG. 7H shows construct 6704 (2X35S-5′UTR nbSEP44-SpPDI-H1 A-Mich-45-2015-CPMV 3′UTR/NOS); Sp: signal peptide.

FIG. 8 shows constructs encoding H3-A/Singapore/19/0019-16: FIG. 8A shows construct 4008 (2X35S-5′UTR CPMV 160-SpPDI-H3 A-Sing-19-0019-16-CPMV 3′UTR/NOS); FIG. 8B shows construct 6134 (2X35S-5′UTR nbMT78-SpPDI-H3 A-Sing-19-0019-16-CPMV 3′UTR/NOS); FIG. 8C shows construct 6136 (2X35S-5′UTR nbHSP69-SpPD1-H3 A-Sing-19-0019-16-CPMV 3′UTR/NOS); FIG. 8D shows construct 6705 (2X35S-5′UTR nbEPI42-SpPD1-H3 A-Sing-19-0019-16-CPMV 3′UTR/NOS); FIG. 8E shows construct 6706 (2X35S-5′UTR nbSNS46-SpPD1-H3 A-Sing-19-0019-16-CPMV 3′UTR/NOS); FIG. 8F shows construct 6707 (2X35S-5′UTR nbCSY65-SpPD1-H3 A-Sing-19-0019-16-CPMV 3′UTR/NOS); FIG. 8G shows construct 6708 (2X35S-5′UTR nbHEL40-SpPD1-H3 A-Sing-19-0019-16-CPMV 3′UTR/NOS); FIG. 8H shows construct 6709 (2X35S-5′UTR nbSEP44-SpPD1-H3 A-Sing-19-0019-16-CPMV 3′UTR/NOS).

FIG. 9A shows the nucleic acid sequence of nbEPI42 (SEQ ID NO:1);

FIG. 9B shows the nucleic acid sequence of nbSNS46 (SEQ ID NO:2);

FIG. 9C shows the nucleic acid sequence of nbCSY65 (SEQ ID NO:3);

FIG. 9D shows the nucleic acid sequence of nbHEL40 (SEQ ID NO:4);

FIG. 9E shows the nucleic acid sequence of nbSEP44 (SEQ ID NO:5);

FIG. 9F shows the nucleic acid sequence of CPMV 160 (SEQ ID NO:6; prior art; described in WO 2015/103704); FIG. 9G shows the nucleic acid sequence of nbMT78 (SEQ ID NO:7; described in U.S. Provisional Application No. 62/643,053 Filed Mar. 14, 2018); FIG. 9H shows the nucleic acid sequence of atHSP69 (SEQ ID NO:8; described in U.S. Provisional Application No. 62/643,053 Filed Mar. 14, 2018).

FIG. 10 shows the nucleic acid sequences pertaining to Dasher constructs: FIG. 10A shows the nucleic acid sequence of IF-(2X35S+C)_CPMV160.c (SEQ ID NO:9); FIG. 10B shows the nucleic acid sequence of IF-Dasher (27-609).r (SEQ ID NO:10); FIG. 10C shows the amino acid sequence of CPMV 160 5′UTR-Dasher DNA sequence (SEQ ID NO:11); FIG. 10D shows the nucleic acid sequence of Dasher DNA sequence (SEQ ID NO:12); FIG. 10E shows the amino acid sequence of Dasher protein sequence (SEQ ID NO:13); FIG. 10F shows the nucleic acid sequence of IF-nbMT78.c (SEQ ID NO:14); FIG. 10G shows the nucleic acid sequence of nbMT78_Dasher.c (SEQ ID NO:15); FIG. 10H shows the nucleic acid sequence of atHSP69_Dasher.c (SEQ ID NO: 16); FIG. 10I shows the nucleic acid sequence of IF-atHSP69.c (SEQ ID NO: 17); FIG. 10J shows the nucleic acid sequence of nbEPI42+Dasher.c (SEQ ID NO: 18); FIG. 10K shows the nucleic acid sequence of IF-nbEPI42.c (SEQ ID NO: 19); FIG. 10L shows the nucleic acid sequence of nbSNS46+Dasher.c (SEQ ID NO: 20); FIG. 10M shows the nucleic acid sequence of IF-nbSNS46.c (SEQ ID NO: 21); FIG. 10N shows the nucleic acid sequence of nbCSY65+Dasher.c (SEQ ID NO: 22); FIG. 100 shows the nucleic acid sequence of IF-nbCSY65.c (SEQ ID NO: 23); FIG. 10P shows the nucleic acid sequence of nbHEL40+Dasher.c (SEQ ID NO: 24); FIG. 10Q shows the nucleic acid sequence of IF-nbHEL40.c (SEQ ID NO: 25); FIG. 10R shows the nucleic acid sequence of nbSEP44+Dasher.c (SEQ ID NO: 26); FIG. 10S shows the nucleic acid sequence of IF-nbSEP44.c (SEQ ID NO: 27).

FIG. 11 shows the nucleic acid sequence relating to GII.4 Syd 12 VP1 constructs: FIG. 11A shows the nucleic acid sequence of IF-GII4Syd12VP1.r (SEQ ID NO: 28); FIG. 11B shows the nucleic acid sequence of CPMV 160 5′UTR-VP1 (GII.4) DNA sequence (SEQ ID NO:29); FIG. 11C shows the nucleic acid sequence of VP1 (GII.4) DNA sequence (SEQ ID NO:30); FIG. 11D shows the amino acid sequence of VP1 (GII.4) protein sequence (SEQ ID NO:31); FIG. 11E shows the nucleic acid sequence of nbMT78+GII4Syd12.c (SEQ ID NO:32); FIG. 11F shows the nucleic acid sequence of atHSP69+GII4Syd12.c (SEQ ID NO:33); FIG. 11G shows the nucleic acid sequence of nbEPI42+GII4Syd12.c (SEQ ID NO:34); FIG. 11H shows the nucleic acid sequence of nbSNS46+GII4Syd12.c (SEQ ID NO:35); FIG. 11I shows the nucleic acid sequence of nbCSY65+GII4Syd12.c (SEQ ID NO:36); FIG. 11J shows the nucleic acid sequence of nbHEL40+GII4Syd12.c (SEQ ID NO:37); FIG. 11K shows the nucleic acid sequence of nbSEP44+GII4Syd12.c (SEQ ID NO:38).

FIG. 12 shows the nucleic acid sequence relating to Rituximab LC and HC constructs: FIG. 12A shows the nucleic acid sequence of IF**-HC(Ritux).s1-6r (SEQ ID NO:39); FIG. 12B shows the nucleic acid sequence of CPMV 160 5′UTR-PDI+Rituximab HC DNA sequence (SEQ ID NO:40); FIG. 12C shows the nucleic acid sequence of PDI+Rituximab HC DNA sequence (SEQ ID NO:41); FIG. 12D shows the amino acid sequence of PDI+Rituximab HC protein sequence (SEQ ID NO:42); FIG. 12E shows the nucleic acid sequence of IF**-LC (Ritux).s1-6r (SEQ ID NO:43); FIG. 12F shows the nucleic acid sequence of CPMV 160 5′UTR-PDI+Rituximab LC DNA sequence (SEQ ID NO:44); FIG. 12G shows the nucleic acid sequence of PDI+Rituximab LC DNA sequence (SEQ ID NO:45); FIG. 12H shows the amino acid sequence of PDI+Rituximab LC protein sequence (SEQ ID NO:46); FIG. 12I shows the nucleic acid sequence of nbMT78_SpPDI.c (SEQ ID NO:47); FIG. 12J shows the nucleic acid sequence of atHSP69_SpPDI.c (SEQ ID NO:48); FIG. 12K shows the nucleic acid sequence of nbEPI42+PDI.c (SEQ ID NO:49); FIG. 12L shows the nucleic acid sequence of nbSNS46+PDI.c (SEQ ID NO:50); FIG. 12M shows the nucleic acid sequence of nbCSY65+PDI.c (SEQ ID NO:51); FIG. 12N shows the nucleic acid sequence of nbHEL40+PDI.c (SEQ ID NO:52); FIG. 12O shows the nucleic acid sequence of nbSEP44+PDI.c (SEQ ID NO:53).

FIG. 13 shows the nucleic acid sequence relating to H1 a Michigan 45/2015 constructs: FIG. 13A shows the nucleic acid sequence of IF-H1cTMCT.s1-4r (SEQ ID NO:54); FIG. 13B shows the nucleic acid sequence of CPMV 160 5′UTR-PDI+H1 Mich DNA sequence (SEQ ID NO:55); FIG. 13C shows the nucleic acid sequence of PDI+H1 Mich DNA sequence (SEQ ID NO:56); FIG. 13D shows the amino acid sequence of PDI+H1 Mich protein sequence (SEQ ID NO:57).

FIG. 14 shows the nucleic acid sequence relating to H3 A/Singapore 19-0019-16 constructs: FIG. 14A shows the nucleic acid sequence of IF-H3Minn15.r (SEQ ID NO:58); FIG. 14B shows the nucleic acid sequence of CPMV 160 5′UTR-PDI+H3 sing DNA sequence (SEQ ID NO:59); FIG. 14C shows the nucleic acid sequence of PDI+H3 sing DNA sequence (SEQ ID NO:60); FIG. 14D shows the amino acid sequence of PDI+H3 sing protein sequence (SEQ ID NO:61).

FIG. 15 shows the nucleic acid sequence of cloning vectors. constructs 1666, 4467, 4160 and 4170: FIG. 15A shows the nucleic acid sequence of cloning vector 1666 from left to right T-DNA (SEQ ID NO:62); FIG. 15B shows the nucleic acid sequence of construct 4467 from 2X35S promoter to NOS terminator (SEQ ID NO:63); FIG. 15C shows the nucleic acid sequence of cloning vector 4160 from left to right T-DNA (SEQ ID NO:64); FIG. 15D shows the nucleic acid sequence of cloning vector 4170 from left to right T-DNA (SEQ ID NO:65).

FIG. 16A shows construct 1666; FIG. 16B shows construct 4160; FIG. 16C shows construct 4170.

### DETAILED DESCRIPTION

The following description is of a preferred embodiment.

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a use or method, denotes that additional elements and/or method steps may be present, but that these additions do not materially affect the manner in which the recited method or use functions. The term “consisting of” when used herein in connection with a use or method, excludes the presence of additional elements and/or method steps. A use or method described herein as comprising certain elements and/or steps may also, in certain embodiments, consist essentially of those elements and/or steps, and in other embodiments consist of those elements and/or steps, whether or not these embodiments are specifically referred to. In addition, the use of the singular includes the plural, and “or” means “and/or” unless otherwise stated. The term “plurality” as used herein means more than one, for example, two or more, three or more, four or more, and the like. Unless otherwise defined herein, all technical and scientific terms used herein have the same meaning as commonly understood by one of ordinary skill in the art. As used herein, the term “about” refers to an approximately +/−10% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

The term “plant”, “portion of a plant”, “plant portion', “plant matter”, “plant biomass”, “plant material”, plant extract”, or “plant leaves”, as used herein, may comprise an entire plant, tissue, cells, or any fraction thereof, intracellular plant components, extracellular plant components, liquid or solid extracts of plants, or a combination thereof, that are capable of providing the transcriptional, translational, and post-translational machinery for expression of one or more than one nucleic acids described herein, and/or from which an expressed protein of interest or VLP may be extracted and purified. Plants may include, but are not limited to, agricultural crops including for example canola, Brassica spp., maize, Nicotiana spp., (tobacco) for example, Nicotiana benthamiana, Nicotiana rustica, Nicotiana, tabacum, Nicotiana alata, Arabidopsis thaliana, alfalfa, potato, sweet potato (Ipomoea batatus), ginseng, pea, oat, rice, soy bean, wheat, barley, sunflower, cotton, com, rye (Secale cereale), sorghum (Sorghum bicolor, Sorghum vulgare), safflower (Carthamus tinctorius).

The term “plant portion”, as used herein, refers to any part of the plant including but not limited to leaves, stem, root, flowers, fruits, callus tissue or cell cultured plant tissue, a cluster of plant cells, a plant cell, for example a plant cell, cluster of plants cells callus or cultured plant tissue obtained from leaves, stem, root, flowers, fruits, a plant extract obtained from leaves, stem, root, flowers, fruits, or a combination thereof. The term plant cell refers to a cell of plant that is bounded by a plasma membrane and may or may not comprise a cell wall. A plant cell includes a protoplast (or spheroplast) that comprises an enzymatically digested cell and that may be obtained using techniques well known in the art (e.g. Davey M R et al., 2005, Biotechnology Advances 23:131-171; which is incorporated herein by reference). Callus plant tissue or cultured plant tissue may be produced using methods well known in the art (e.g. M K Razdan 2nd Ed., Science Publishers, 2003; which is incorporated herein by reference) The term “plant extract”, as used herein, refers to a plant-derived product that is obtained following treating a plant, a portion of a plant, a plant cell, or a combination thereof, physically (for example by freezing followed by extraction in a suitable buffer), mechanically (for example by grinding or homogenizing the plant or portion of the plant followed by extraction in a suitable buffer), enzymatically (for example using cell wall degrading enzymes), chemically (for example using one or more chelators or buffers), or a combination thereof. A plant extract may be further processed to remove undesired plant components for example cell wall debris. A plant extract may be obtained to assist in the recovery of one or more components from the plant, portion of the plant or plant cell, for example a protein (including protein complexes, protein surprastructures and/or VLPs), a nucleic acid, a lipid, a carbohydrate, or a combination thereof from the plant, portion of the plant, or plant cell. If the plant extract comprises proteins, then it may be referred to as a protein extract. A protein extract may be a crude plant extract, a partially purified plant or protein extract, or a purified product, that comprises one or more proteins, protein complexes, protein suprastructures, and/or VLPs, from the plant tissue. If desired a protein extract, or a plant extract, may be partially purified using techniques known to one of skill in the art, for example, the extract may be subjected to salt or pH precipitation, centrifugation, gradient density centrifugation, filtration, chromatography, for example, size exclusion chromatography, ion exchange chromatography, affinity chromatography, or a combination thereof. A protein extract may also be purified, using techniques that are known to one of skill in the art.

By “nucleotide (or nucleic acid) sequence of interest”, or “coding region of interest”, it is meant any nucleotide sequence, or coding region (these terms may be used interchangeably) that is to be expressed within a plant, portion of a plant, or a plant cell, to produce a protein of interest. Such a nucleotide sequence of interest may encode, but is not limited to, native or modified proteins, an industrial enzyme or a modified industrial enzyme, an agricultural protein or a modified agricultural protein, a helper protein, a protein supplement, a pharmaceutically active protein, a nutraceutical, a value-added product, or a fragment thereof for feed, food, or both feed and food use.

The protein of interest may comprise a native, or a non-native signal peptide; the non-native signal peptide may be of plant origin. For example, which is not to be considered limiting, the non-native signal peptide may be obtained from alfalfa protein disulfide isomerase (PDI SP; nucleotides 32-103 of Accession No. Z11499), potato patatin (PatA SP; located nucleotides 1738-1806 of GenBank Accession number A08215), Kiwi actinidin (Act), Tobacco cysteine proteinase 3 precursor (CP23), Com AZein (AZein), Papaya proteinase I (Papain; Pap) and Thale cress cysteine proteinase RD21A (RD21). The native signal peptide may correspond to that of the protein of interest being expressed.

The nucleotide sequence of interest, or coding region of interest may also include a nucleotide sequence that encodes a pharmaceutically active protein, for example growth factors, growth regulators, antibodies, antigens, and fragments thereof, or their derivatives useful for immunization or vaccination and the like. Such proteins include, but are not limited to a protein that is a human pathogen, a viral protein, for example but not limited to virus like particle (VLP)-forming antigens, one or more proteins from Norovirus, Respiratory syncytial virus (RSV), Rotavirus, influenza virus, human immunodeficiency virus (HIV), Rabies virus, human papiloma virus (HPV), Enterovirus 71 (EV71), or interleukins, for example one or more than one of IL-1 to IL-24, IL-26 and IL-27, cytokines, Erythropoietin (EPO), insulin, G-CSF, GM-CSF, hPG-CSF, M-CSF or combinations thereof, interferons, for example, interferon-alpha, interferon-beta, interferon-gama, blood clotting factors, for example, Factor VIII, Factor IX, or tPA hGH, receptors, receptor agonists, antibodies for example but not limited to rituximab, neuropolypeptides, insulin, vaccines, growth factors for example but not limited to epidermal growth factor, keratinocyte growth factor, transformation growth factor, growth regulators, antigens, autoantigens, fragments thereof, or combinations thereof.

The protein of interest may include an influenza hemagglutinin (HA; see WO 2009/009876, WO 2009/076778, WO 2010/003225, which are incorporated herein by reference). HA is a homotrimeric membrane type I glycoprotein, generally comprising a signal peptide, an HA1 domain, and an HA2 domain comprising a membrane-spanning anchor site at the C-terminus and a small cytoplasmic tail. Nucleotide sequences encoding HA are well known and are available (see, for example, the BioDefense and Public Health Database (Influenza Research Database; Squires et al., 2008, Nucleic Acids Research 36:D497-D503) at URL: biohealthbase.org/GSearch/home.do?decorator=Influenza; or the databases maintained by the National Center for Biotechnology Information (see URL: ncbi.nlm.nih.gov), both of which are incorporated herein by reference).

An HA protein may be of a type A influenza, a type B influenza, or is a subtype of type A influenza HA selected from the group of H1, H2, H3, H4, H5, H6, H7, H8, H9, H10, H11, H12, H13, H14, H15, H16. H17 and H18. In some aspects of the invention, the HA may be from a type A influenza, selected from the group H1, H2, H3, H5, H6, H7 and H9. Fragments of the HAs listed above may also be considered a protein of interest. Furthermore, domains from an HA type or subtype listed above may be combined to produce chimeric HA's (see for example WO2009/076778 which is incorporated herein by reference).

Examples of subtypes comprising HA proteins include A/New Caledonia/20/99 (H1N1), A/Indonesia/5/2006 (H5N1), A/PuertoRico/8/34 (H1N1), A/Brisbane/59/2007 (H1N1), A/Solomon Islands 3/2006 (H1N1), A/California/04/2009 (H1N1), A/California/07/2009 (H1N1), A/chicken/New York/1995, A/Singapore/1/57 (H2N2), A/herring gull/DE/677/88 (H2N8), A/Texas/32/2003, A/mallard/MN/33/00, A/duck/Shanghai/1/2000, A/northern pintail/TX/828189/02, A/Brisbane 10/2007 (H3N2), A/Wisconsin/67/2005 (H3N2), A/Victoria/361/2011 (H3N2), A/Texas/50/2012 (H3N2), A/Hawaii/22/2012 (H3N2), A/New York/39/2012 (H3N2), A/Perth/16/2009 (H3N2), C/Johannesburg/66,A/Anhui/1/2005 (H5N1), A/Vietnam/1194/2004 (H5N1), A/Teal/HongKong/W312/97 (H6N1), A/Equine/Prague/56 (H7N7), A/Turkey/Ontario/6118/68 (H8N4), H7 A/Hangzhou/1/2013, A/Anhui/1/2013 (H7N9), A/Shanghai/2/2013 (H7N9), A/shoveler/Iran/G54/03, A/HongKong/1073/99 (H9N2), A/chicken/Germany/N/1949 (H10N7), A/duck/England/56 (H1IN6), A/duck/Alberta/60/76 (H12N5), A/Gull/Maryland/704/77 (H13N6), A/Mallard/Gurjev/263/82, A/duck/Australia/341/83 (H15N8), A/black-headed gull/Sweden/5/99 (H16N3), B/Malaysia/2506/2004, B/Florida/4/2006, B/Brisbane/60/08, B/Massachusetts/2/2012-like virus (Yamagata lineage), B/Wisconsin/1/2010 (Yamagata lineage), or B/Lee/40.

The HA may also be a modified or chimeric HA, for example, the native transmembrane domain of the HA may be replaced with a heterologous transmembrane domain (WO 2010/148511; which is incorporated herein by reference), the HA may comprise a chimeric ectodomain (WO2012/083445, which is incorporated herein by reference), or the HA may comprises a proteolytic loop deletion (WO 2014/153647, which is incorporated herein by reference).

The protein of interest may also include a norovirus protein or a modified norovirus protein as described in WO2018/170603; which is incorporated herein by reference), or U.S. provisional application 62/593,006 (filed Nov. 11, 2017; which is incorporated herein by reference). Norovirus is a non-enveloped viral strain of the genus norovirus of the family Caliciviridae that is characterized as having a single-stranded, positive-sense RNA. Norovirus strains may include any known norovirus strain, but also modifications to known norovirus strains that are known to develop on a regular basis over time. For example, norovirus strains may include, GI.1, GI.2, GI.3, GI.5, GI.7, GII.1, GII.2, GII.3, GII.4, GII.5, GII.6, GII.7, GII.12, GII.13, GII.14, GII.17 and GII.21, for example but not limited to Hu/GI.1/United States/Norwalk/1968, Hu/GI.2/Leuven/2003/BEL, Hu/GI.3/S29/2008/Lilla Edet/Sweden, Hu/GI.5/Siklos/Hun5407/2013/HUN, Hu/GII.1/Ascension208/2010/USA, Hu/GII.2/CGMH47/2011/TW, Hu/GII.3/Jingzhou/2013402/CHN, Hu/GII.4/Sydney/NSW0514/2012/AU, US96/GII.4/Dresden174/1997/DE_AY741811, FH02/GII.4/FarmingtonHills/2002/US_AY502023, Hnt04; GII.4/Hunter-NSW504D/2004/AU_DQ078814, 2006b: GII.4/Shellharbour-NSW696T/2006/AU_EF684915, NO09: GII.4/Orange-NSW001P/2008/AU_GQ845367, Hu/GII.5/AlbertaEI390/2013/CA, Hu/GII.6/Ohio/490/2012/USA, GII.7/Musa/2010/A1173774, Hu/GII.12/HS206/2010/USA, GII.13/VA173/2010/H9AWU4, GII.14_Saga_2008_JPN_ADE28701 native VP1, Hu/GII.17/Kawasaki323/2014/JP, and Hu/GII.21/Salisbury 150/2011/USA. Norovirus strains also include strains having from about 30-100% or any amount therebetween, amino acid sequence identity, to the VP1 protein, the VP2 protein, or both the VP1 and the VP2 proteins, with any of the above norovirus strains.

The terms “percent similarity”, “sequence similarity”, “percent identity”, or “sequence identity”, when referring to a particular sequence, are used for example as set forth in the University of Wisconsin GCG software program, or by manual alignment and visual inspection (see, e.g., Current Protocols in Molecular Biology, Ausubel et al., eds. 1995 supplement). Methods of alignment of sequences for comparison are well-known in the art. Optimal alignment of sequences for comparison can be conducted, using for example the algorithm of Smith & Waterman, (1981, Adv. Appl. Math. 2:482), by the alignment algorithm of Needleman & Wunsch, (1970, J. Mol. Biol. 48:443), by the search for similarity method of Pearson & Lipman, (1988, Proc. Natl. Acad. Sci. USA 85:2444), by computerized implementations of these algorithms (for example: GAP, BESTFIT, FASTA, and TFASTA in the Wisconsin Genetics Software Package, Genetics Computer Group (GCG), 575 Science Dr., Madison, Wis.).

An example of an algorithm suitable for determining percent sequence identity and sequence similarity are the BLAST and BLAST 2.0 algorithms, which are described in Altschul et al., (1977, Nuc. Acids Res. 25:3389-3402) and Altschul et al., (1990, J. Mol. Biol. 215:403-410), respectively. BLAST and BLAST 2.0 are used, with the parameters described herein, to determine percent sequence identity for the nucleic acids and proteins of the invention. For example the BLASTN program (for nucleotide sequences) may use as defaults a wordlength (W) of 11, an expectation (E) of 10, M=5, N=−4 and a comparison of both strands. For amino acid sequences, the BLASTP program may use as defaults a word length of 3, and expectation (E) of 10,and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands. Software for performing BLAST analyses is publicly available through the National Center for Biotechnology Information (see URL: ncbi.nlm.nih.gov/).

The term “nucleic acid segment” as used herein refers to a sequence of nucleic acids that encodes a protein of interest. In addition to the sequence of nucleic acids, the nucleic acid segment comprise a regulatory region and a terminator that are operatively linked to the sequence of nucleic acids. The regulatory region may comprise a promoter, and an enhancer element (expression enhancer) operatively linked to the promoter.

The term “nucleic acid complex” as used herein refers to a combination of two or more than two nucleic acid segments. The two or more than two nucleic acid segments may be present in a single nucleic acid, so that the nucleic acid complex comprises two, or more than two nucleic acid segments, with each nucleic acid segment under the control of a regulatory region and a terminator. Alternatively, the nucleic acid complex may comprise two or more separate nucleic acids, each of the nucleic acids comprising one or more than one nucleic acid segment, where each nucleic acid segment is under the control of a regulatory region and a terminator. For example a nucleic acid complex may comprise one nucleic acid that comprises two nucleic acid segments, a nucleic acid complex may comprise two nucleic acids, each nucleic acid comprising one nucleic acid segment, or a nucleic acid complex may comprise two or more than two nucleic acids, with each nucleic acid comprising one or more than one nucleic acid segment.

The terms “vector” or “expression vector” as used herein, refer to a recombinant nucleic acid for transferring exogenous nucleic acid sequences into host cells (e.g. plant cells) and directing expression of the exogenous nucleic acid sequences in the host cells. The vector may be introduced to the plant, the portion of the plant, or a plant cell, directly, or the vectors may be introduced in the plant, the portion of the plant, or a plant cell as part of a plant expression system. The vector or expression vector comprise a construct or an expression construct. The construct or expression construct comprises a nucleotide sequence comprising a nucleic acid of interest under the control of, and operably (or operatively) linked to, an appropriate promoter, an expression enhancer, or other regulatory elements for transcription of the nucleic acid of interest in a host cell. As one of skill in the art would appreciate, the construct or expression cassette may comprise a termination (terminator) sequence that is any sequence that is active in the plant host. For example the termination sequence may be derived from the RNA-2 genome segment of a bipartite RNA virus, e.g. a comovirus, the termination sequence may be a NOS terminator, the terminator sequence may be obtained from the 3′UTR of the alfalfa plastocyanin gene, or a combination thereof.

The constructs of the present disclosure may further comprise a 3′ untranslated region (UTR). A 3′ untranslated region contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soy bean storage protein genes, the small subunit of the ribulose-1, 5-bisphosphate carboxylase gene (ssRUBISCO; U.S. Pat. No. 4,962,028; which is incorporated herein by reference), the promoter and/or terminator used in regulating plastocyanin expression.

For example, which is not to be considered limiting, a CPMV 3′UTR+NOS terminator may be used as a 3′UTR sequence that is operatively linked to the 3′ end of the nucleic acid sequence encoding the protein of interest.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a nucleotide sequence of interest, this may result in expression of the nucleotide sequence of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element may comprise a basal promoter element, responsible for the initiation of transcription, as well as other regulatory elements that modify gene expression.

There are several types of regulatory regions, including those that are developmentally regulated, inducible or constitutive. A regulatory region that is developmentally regulated or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example seed-specific a regulatory region, include the napin promoter, and the cruciferin promoter (Rask et al., 1998, J. Plant Physiol. 152:595-599; Bilodeau et al., 1994, Plant Cell 14:125-130). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically, the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I.R.P., 1998, Trends Plant Sci. 3, 352-358). Examples, of potential inducible promoters include, but not limited to, tetracycline-inducible promoter (Gatz, C., 1997, Ann. Rev. Plant Physiol. Plant Mol. Biol. 48, 89-108), steroid inducible promoter (Aoyama, T. and Chua, N. H., 1997, Plant J. 2, 397-404) and ethanol-inducible promoter (Salter, M. G., et al, 1998, Plant Journal 16, 127-132; Caddick, M. X., et al, 1998, Nature Biotech. 16, 177-180) cytokinin inducible IB6 and CKII genes (Brandstatter, I. and Kieber, J. J., 1998, Plant Cell 10, 1009-1019; Kakimoto, T., 1996, Science 274, 982-985) and the auxin inducible element, DR5 (Ulmasov, T., et al., 1997, Plant Cell 9, 1963-1971).

A constitutive regulatory region directs the expression of a gene throughout the various parts of a plant and continuously throughout plant development. Examples of known constitutive regulatory elements include promoters associated with the CaMV 35S transcript. (p35S; Odell et al., 1985, Nature, 313:810-812; which is incorporated herein by reference), the rice actin 1 (Zhang et al, 1991, Plant Cell, 3:1155-1165), actin 2 (An et al., 1996, Plant J., 10:107-121), or tms 2 (U.S. Pat. No. 5,428,147), and triosephosphate isomerase 1 (Xu et. al., 1994, Plant Physiol. 106:459-467) genes, the maize ubiquitin 1 gene (Cornejo et al, 1993, Plant Mol. Biol. 29:637-646), the Arabidopsis ubiquitin 1 and 6 genes (Holtorf et al, 1995, Plant Mol. Biol. 29:637-646), the tobacco translational initiation factor 4A gene (Mandel et al, 1995 Plant Mol. Biol. 29:995-1004); the Cassava Vein Mosaic Virus promoter, pCAS, (Verdaguer et al., 1996); the promoter of the small subunit of ribulose biphosphate carboxylase, pRbcS: (Outchkourov et al., 2003), the pUbi (for monocots and dicots).

The term “constitutive” as used herein does not necessarily indicate that a nucleotide sequence under control of the constitutive regulatory region is expressed at the same level in all cell types, but that the sequence is expressed in a wide range of cell types even though variation in abundance is often observed.

The constructs or expression constructs as described above may be present in a vector (or an expression vector). The vector may comprise border sequences which permit the transfer and integration of the expression cassette into the genome of the organism or host. The construct may be a plant binary vector, for example a binary transformation vector based on pPZP (Hajdukiewicz, et al. 1994). Other example constructs include pBin19 (see Frisch, D. A., L. W. Harris-Haller, et al. 1995, Plant Molecular Biology 27:405-409).

A nucleotide sequence interest that encodes a protein requires the presence of a “translation initiation site” or “initiation site” or “translation start site” or “start site” or “start codon” located upstream of the gene to be expressed. Such initiation sites may be provided either as part of an enhancer sequence or as part of a nucleotide sequence encoding the protein of interest.

The term “native”, “native protein” or “native domain”, as used herein, refers to a protein or domain having a primary amino acid sequence identical to wildtype. Native proteins or domains may be encoded by nucleotide sequences having 100% sequence similarity to the wildtype sequence. A native amino acid sequence may also be encoded by a human codon (hCod) optimized nucleotide sequence or a nucleotide sequence comprising an increased GC content when compared to the wild type nucleotide sequence provided that the amino acid sequence encoded by the hCod-nucleotide sequence exhibits 100% sequence identity with the native amino acid sequence.

By a nucleotide sequence that is “human codon optimized” or an “hCod” nucleotide sequence, it is meant the selection of appropriate DNA nucleotides for the synthesis of an oligonucleotide sequence or fragment thereof that approaches the codon usage generally found within an oligonucleotide sequence of a human nucleotide sequence. By “increased GC content” it is meant the selection of appropriate DNA nucleotides for the synthesis of an oligonucleotide sequence or fragment thereof in order to approach codon usage that, when compared to the corresponding native oligonucleotide sequence, comprises an increase of GC content, for example, from about 1 to about 30%, or any amount therebetween, over the length of the coding portion of the oligonucleotide sequence. For example, from about 1, 2, 4, 6, 8, 10, 12, 14, 16, 18, 20, 22, 24, 26, 28, 30%, or any amount therebetween, over the length of the coding portion of the oligonucleotide sequence. As described below, a human codon optimized nucleotide sequence, or a nucleotide sequence comprising an increased GC content (when compared to the wild type nucleotide sequence) exhibits increased expression within a plant, portion of a plant, or a plant cell, when compared to expression of the non-human optimized (or lower GC content) nucleotide sequence.

The term “single construct” or “single constructs”, as used herein, refers to a nucleic acid comprising a single nucleic acid sequence. The term “dual construct” or “dual constructs”, as used herein, refers to a nucleic acid comprising two nucleic acid sequences.

By co-expression it is meant the introduction and expression of two or more nucleotide sequences, each of the two or more nucleotide sequences encoding a protein of interest, or a fragment of a protein of interest within a plant, portion of a plant or a plant cell. The two or more nucleotide sequences may be introduced into the plant, portion of the plant or the plant cell within one vector, so that each of the two or more nucleotide sequences is under the control of a separate regulatory region (e.g. comprising a dual construct). Alternatively, the two or more nucleotide sequences may be introduced into the plant, portion of the plant or the plant cell within separate vectors (e.g. comprising single constructs), and each vector comprising appropriate regulatory regions for the expression of the corresponding nucleic acid. For example, two nucleotide sequences, each on a separate vector and introduced into separate Agrobacterium tumefaciens hosts, may be co-expressed by mixing suspensions of each A. tumefaciens host in a desired volume (for example, an equal volume, or the ratios of each A. tumefaciens host may be altered) before vacuum infiltration. In this manner, co-infiltration of multiple A. tumifaciens suspensions permits co-expression of multiple transgenes.

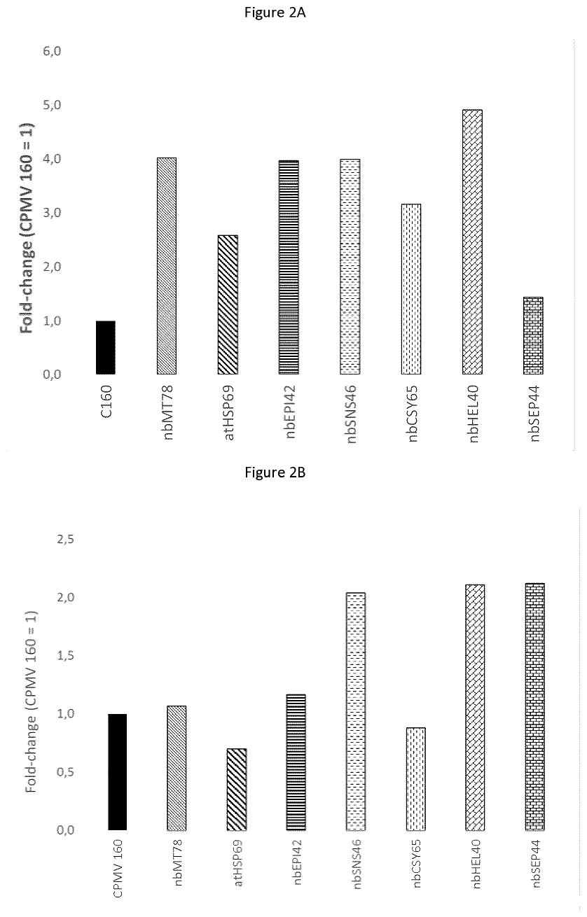

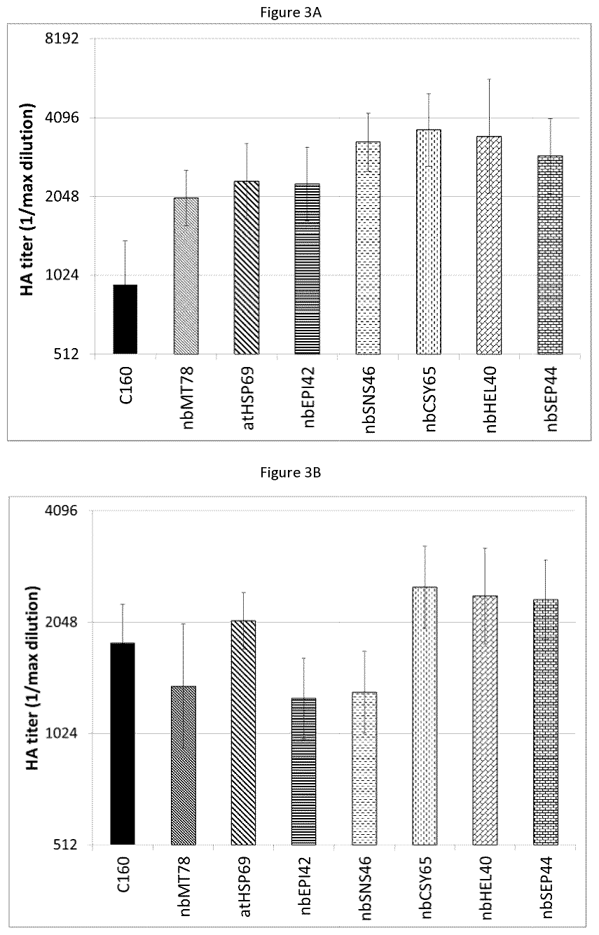

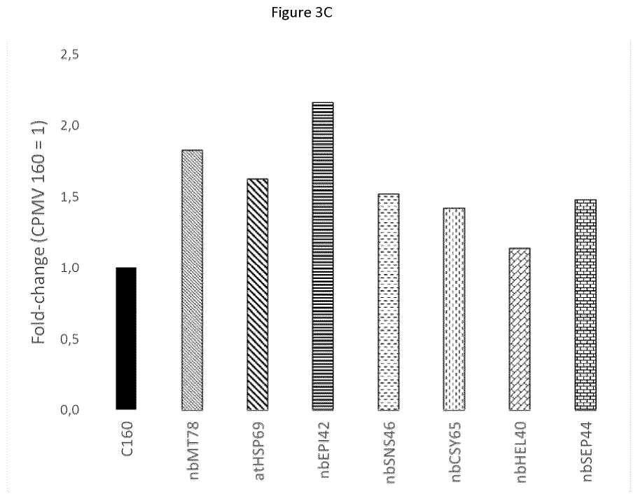

The nucleic acid encoding a protein of interest as described herein may further comprise sequences that enhance expression of the protein of interest in the plant, portion of the plant, or plant cell. Sequences that enhance expression are described herein and for example, may include one or more of, an expression enhancer element obtained from a nucleic acid encoding a secretory protein (SPEE) or an expression enhancer element obtained from a nucleic acid encoding a cytosolic protein (CPEE), in operative association with the nucleic acid encoding the protein of interest. Non-limiting examples of using the expression enhancer as described herein for the expression of a secreted protein includes any protein of interest comprising a signal peptide or signal sequence that targets the protein of interest to the extracellular compartment, for example an antibody (see FIG. 3C), or virus like particles (VLPs) that are known to bud from the plasma membrane, for example, influenza HA (see for example FIGS. 3A and 3B). Non-limiting examples of proteins that are produced cytosolically include any protein of interest that do not comprise a secretory peptide or signal sequence (see for example FIG. 2A), or VLPs that are known to be produced and retained within the cytsol, for example norovirus (see FIG. 2B).

The sequence encoding the protein of interest may also be optimized for human codon usage, increased GC content, or a combination thereof. Co-expression of a nucleic acid encoding a second protein of interest may lead to functional multimeric protein, for example an antibody comprising heavy and light chain components, or to an increased yield of protein. If the protein of interest results in the production of a VLP, then co-expression of two or more proteins may result in an increase yield, increased density, increased integrity, or combination thereof, of the VLPs that comprise the protein of interest. The increase in yield, density, integrity, or combination thereof, may be determined by comparing the yield, density, integrity, or combination thereof, obtained using the expression enhancer as described herein with the yield, density, integrity, or combination thereof, of the same nucleotide sequence encoding the heterologous open reading frame but not operatively linked to an expression enhancer, or for example, when operatively linked to the prior art expression enhancer CPMV 160 (SEQ ID NO:6).

A plant expression system comprising a nucleic acid comprising a regulatory region, operatively linked with one or more than one expression enhancer as described herein and a nucleotide sequence of interest is also provided. The plant expression system may comprise one or more than one vector, one or more than one construct or one or more than one nucleic acid, that comprises the regulatory region operatively linked with one or more than one expression enhancer as described herein and the nucleotide sequence or nucleic acid of interest, along with other components that may be introduced into the plant, the portion of the plant or a plant cell. For example, the plant expression system may also comprise additional vectors, constructs, or nucleic acids, additional Agrobacteria comprising vectors, constructs or nucleic acids for co-expression, one or more than one chemical compound to modify the efficiency of transformation, other components, or a combination thereof.

Furthermore, a nucleic acid comprising a promoter (regulatory region) sequence, operatively linked with an expression enhancer comprising an expression enhancer as described herein, and a nucleotide sequence of interest is described. The nucleic acid may further comprise a sequence encoding a 3′UTR, for example a comovirus 3′ UTR, or a plastocyanin 3′ UTR, and a terminator sequence, for example a NOS terminator, so that the nucleotide sequence of interest is inserted upstream from the 3′UTR.

“Expression enhancer(s)”, “enhancer sequence(s)” or “enhancer element(s)”, as referred to herein, when operatively linked to a nucleic acid of interest, for example a heterologous nucleic acid of interest, results in expression of the nucleic acid of interest. The expression enhancer may also enhance or increase expression of a downstream heterologous open reading frame (ORF) to which they are attached. The expression enhancer may be operatively linked at the 5′end of the enhancer sequence with a regulatory region that is active in a plant, and operatively linked to a nucleotide sequence of interest at the 3′end of the expression enhancer in order to drive expression of the nucleotide sequence of interest within a host, for example a plant, portion of a plant or a plant cell. Expression enhancers described herein include sequences derived from, or that share sequence similarity with, a nucleotide sequence selected from nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5).

By “operatively linked” it is meant that the particular sequences interact either directly or indirectly to carry out an intended function, such as mediation or modulation of expression of a nucleic acid sequence. The interaction of operatively linked sequences may, for example, be mediated by proteins that interact with the operatively linked sequences.

The term “5′UTR” or “5′ untranslated region” or “5′ leader sequence” refers to regions of an mRNA that are not translated. The 5′UTR typically begins at the transcription start site and ends just before the translation initiation site or start codon (usually AUG in an mRNA, ATG in a DNA sequence) of the coding region. The 5′ UTR may modulate the stability and/or translation of an mRNA transcript. If desired, the length of the 5′UTR may be modified by mutation for example substitution, deletion or insertion of the 5′UTR.

The expression enhancer may further comprise one or more “restriction site(s)” or “restriction recognition site(s)”, “multiple cloning site”, “MCS”, “cloning site(s)”, “polylinker sequence”, or “polylinker' to facilitate the insertion of the nucleotide of interest into the plant expression system. Restrictions sites are specific sequence motifs that are recognized by restriction enzymes and are well known in the art. The expression enhancer may comprise one or more restriction sites or cloning sites that are located downstream (3′) of the 5′UTR. The polylinker sequence (multiple cloning site) may comprise any sequence of nucleic acids that are useful for adding and removing nucleic acid sequences, including a nucleotide sequence encoding a protein of interest, to the 3′ end of the 5′UTR. A polylinker sequence may comprise from 4 to about 100 nucleic acids, or any amount therebetween. As would be evident to one of skill in the art, any multiple cloning site (MCS), or an MCS of different length (either shorter or longer) may be used.

Expression systems, or vectors, to produce one or more proteins of interest in a plant using one or more than one expression enhancers as described herein are also provided. The expression systems described herein comprise an expression cassette comprising one or more than one expression enhancer, or a sequence that comprises 80-100% sequence similarity, or any amount therebetween, with the one or more than one expression enhancer. The expression cassette comprising the expression enhancer may further comprise a regulatory region that is active in a plant that is operatively linked to the 5′end of the expression enhancer. A nucleotide sequence of interest may be operatively linked to the 3′end of the expression cassette so that when introduced within a plant, portion of the plant or a plant cell, expression of the nucleotide sequence of interest within a plant is achieved.

Plants, portions of plants, plant cells, plant tissues, whole plants, inoculum, nucleic acids, constructs comprising nucleotide sequences of interest encoding proteins of interest, expression cassettes or expression systems comprising the one or more than one expression enhancer as described herein, and methods of expressing a protein of interest in plants, portions of plants, or plant cells are also provided.

The nucleotide sequence of interest may be fused (operatively linked) to the enhancer sequence comprising a plant regulatory region, using a variety of approaches. For example, which are not to be considered limiting, a nucleotide sequence of interest encoding a protein of interest may be fused to the 3′ end of an expression enhancer immediately after the 5′UTR sequence.

The constructs of the present invention can be introduced into plant cells in a stable or transient manner using Ti plasmids, Ri plasmids, plant virus vectors, direct DNA transformation, micro-injection, electroporation, infiltration, and the like. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison-Wesley, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (Gene 100:247-250 (1991), Scheid et al. (Mol. Gen. Genet. 228:104-112, 1991), Guerche et al. (Plant Science 52:111-116, 1987), Neuhause et al. (Theor. Appl Genet. 75:30-36, 1987), Klein et al., Nature 327:70-73 (1987); Howell et al. (Science 208: 1265, 1980), Horsch et al. (Science 227:1229-1231, 1985), DeBlock et al., Plant Physiology 91:694-701, 1989), Methods for Plant Molecular Biology (Weissbach and Weissbach, eds., Academic Press Inc., 1988), Methods in Plant Molecular Biology (Schuler and Zielinski, eds., Academic Press Inc., 1989), Liu and Lomonossoff (J. Virol Meth, 105:343-348, 2002,), U.S. Pat. Nos. 4,945,050; 5,036,006; 5,100,792; 6,403,865; 5,625,136, (all of which are hereby incorporated by reference).

Transient expression methods may be used to express the constructs of the present invention (see Liu and Lomonossoff, 2002, Journal of Virological Methods, 105:343-348; which is incorporated herein by reference). Alternatively, a vacuum-based transient expression method, as described by Kapila et al. 1997 (incorporated herein by reference) may be used. These methods may include, for example, but are not limited to, a method of Agro-inoculation or Agro-infiltration, however, other transient methods may also be used as noted above. With either Agro-inoculation or Agro-infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis the Agrobacterium infect and transfer t-DNA copies into the cells. The t-DNA is episomally transcribed and the mRNA translated, leading to the production of the protein of interest in infected cells, however, the passage of t-DNA inside the nucleus is transient.

If the nucleotide sequence of interest encodes a product that is directly or indirectly toxic to the plant, then by using the method of the present invention, such toxicity may be reduced throughout the plant by selectively expressing the nucleotide sequence of interest within a desired tissue or at a desired stage of plant development. In addition, the limited period of expression resulting from transient expression may reduce the effect when producing a toxic product in the plant. An inducible promoter, a tissue-specific promoter, or a cell specific promoter may be used to selectively direct expression of the sequence of interest.

U.S. Provisional Application No. 62/643,053 (filed Mar. 14, 2018; which is incorporated herein by reference) describes the identification of 15 plant-derived expression enhancer elements (UTRs) identified using polysomal analysis. Two of these UTRs (nbMT78, SEQ ID NO:7; and atHSP69, SEQ ID NO:8) are included herein for comparative purposes. The expression enhancers described herein (endogenous expression enhancers) were identified by analysing the 5′ region of UTRs that were identified using the polysomal analysis described in U.S. Provisional Application No. 62/643,053. However, the endogenous expression enhancers described herein were not identified within the group of UTRs identified as a result of the polysomal analysis (described in U.S. Provisional Application No. 62/643,053), nor were the endogenous expression enhancers described herein identified using polysomal analysis.

Examples of expression enhancers (endogenous expression enhancers) as described herein include:

```
nbEPI42;
  (SEQ ID NO: 1)
ACTTTAATTTGCTGATTTTCAACAAAATCAAGAATTTCAGCA;
nbSNS46;
  (SEQ ID NO: 2)
ATTCAGTGCTTAACTGGTTATTGAGTAAGTTATCAAAAAGCAAAAA;
nbCSY65;
  (SEQ ID NO: 3)
ACTTTTCTAATCAATCATCAAACAGAACGCAGAAAATTTCCTAAAA
ACAAAAAAAAGGCATACAA;
nbHEL40;
  (SEQ ID NO: 4)
ACTCCATTTGAATCTATCAAACCAAAACACATTGAGCAAA;
nbSEP44;
  (SEQ ID NO: 5)
ACTTCAATCACTCCACACTTTATTCTCTTTCAAAACCTACACTC;
```

The enhancer sequence may be selected from any one of SEQ ID NO's:1-5, or a nucleotide sequence that exhibits 100%, 99%, 98%, 97%, 96%, 95%, or 90%, or any amount therebetween, sequence identity to the sequence as set forth in any one of SEQ ID NO's:1-15, wherein, the expression enhancer, when operatively linked to a nucleic acid of interest, results in the expression of the nucleic acid of interest, or increases the level of expression of the nucleic acid of interest when compared to the level of expression of the same nucleic acid of interest that is not operatively linked to the expression enhancer, or for example, when operatively linked to the prior art expression enhancer CPMV 160 (SEQ ID NO:6). Each of the enhancer sequences shown in SEQ ID NO's:1-5 may be modified using methods known to one of skill in the art, including deletion, insertion, and/or substitution of one or more than one nucleotide of the enhancer sequence, to produce an expression enhancer that results in a similar or increased enhancer activity, or that results in another beneficial property of the expression enhancer (see for example Diamos et.al., Frontiers in Plant Science. 2016, vol 7 pp. 1-15; Dvir S. et. al., 2013, PNAS published online Jul. 15, 2013; Leppek K et. al., 2018, Nature Reviews Mol. Cell Biol. 19:158-174; which are incorporated herein by reference). For example, a beneficial property may include improved transcriptional initiation, improved mRNA stability, improved mRNA translation, or a combination thereof.

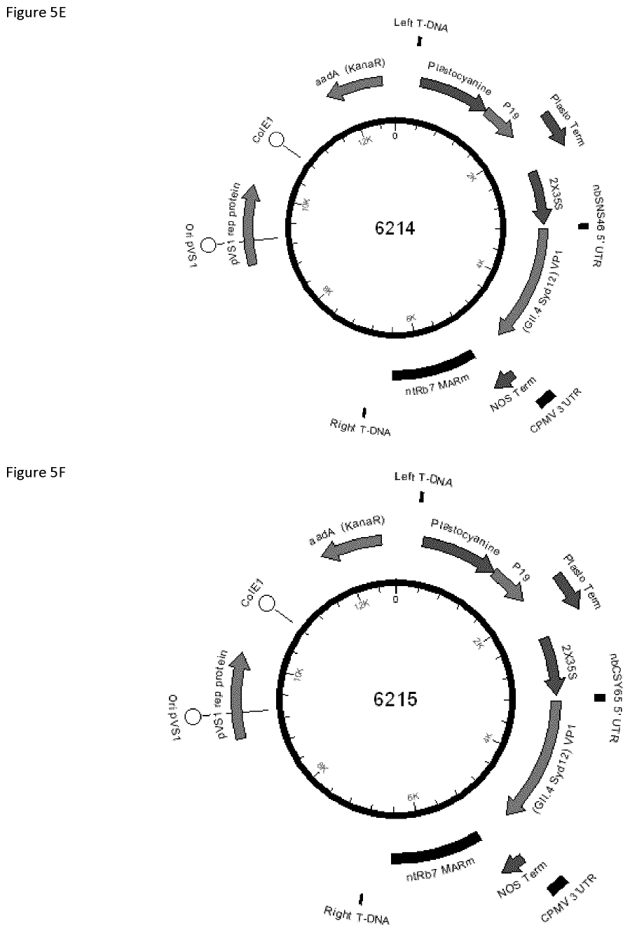

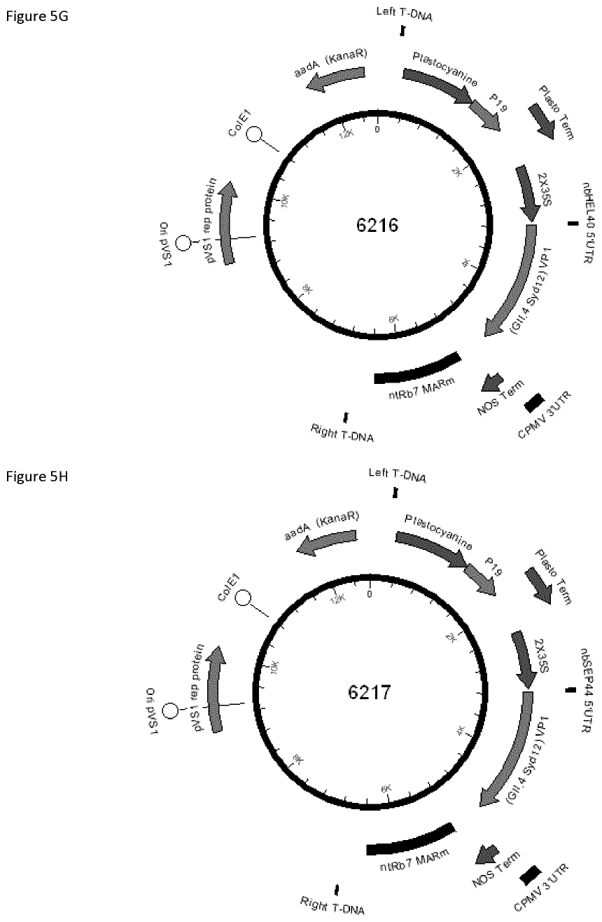

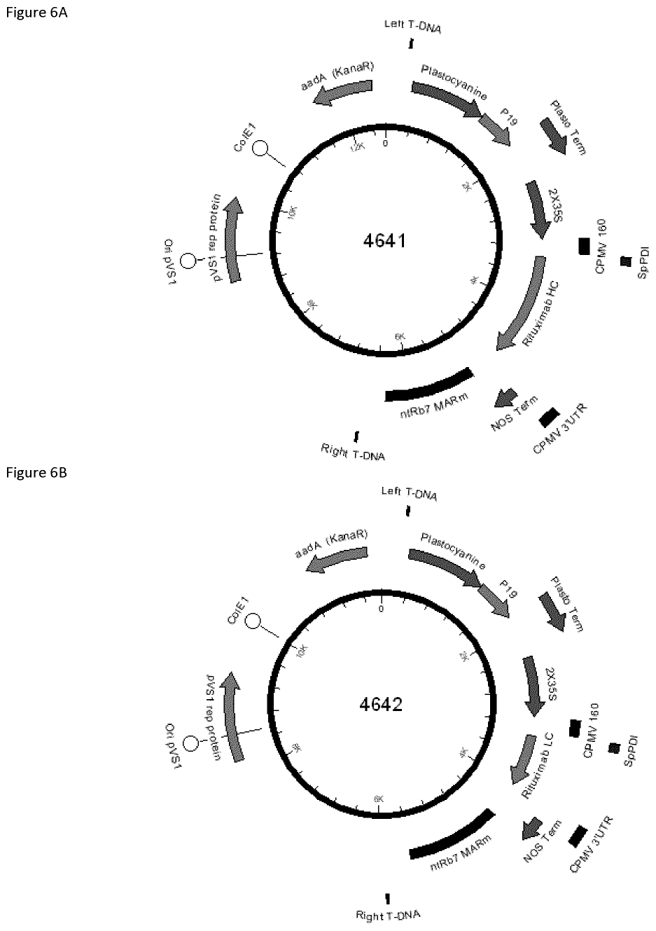

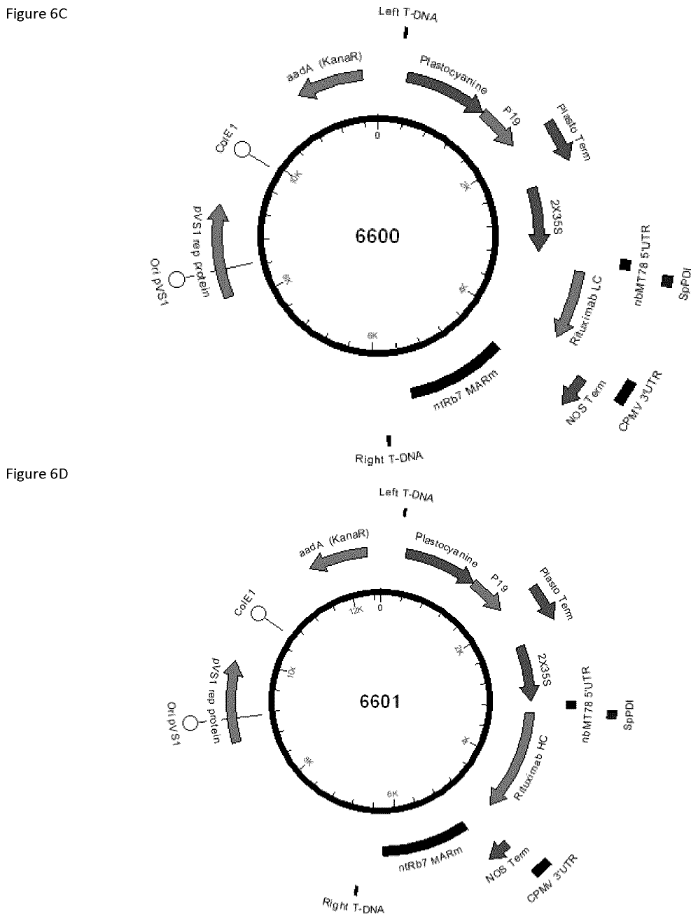

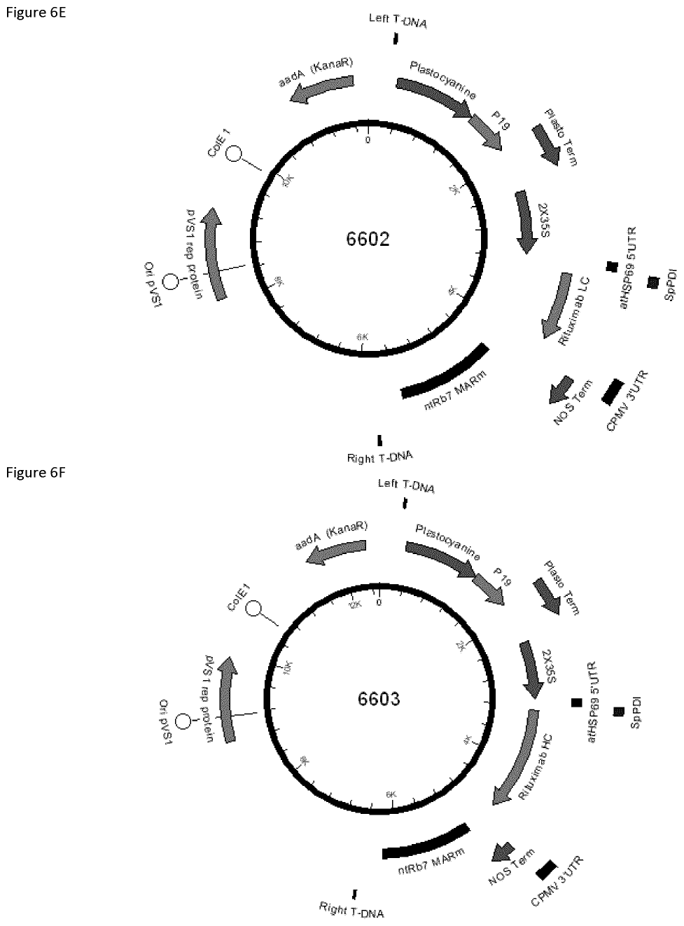

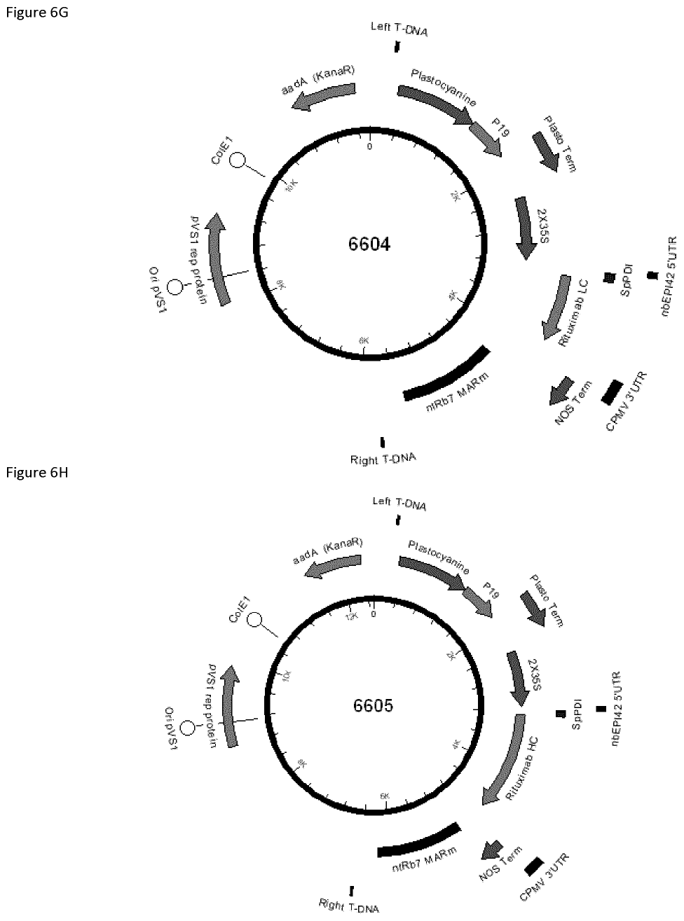

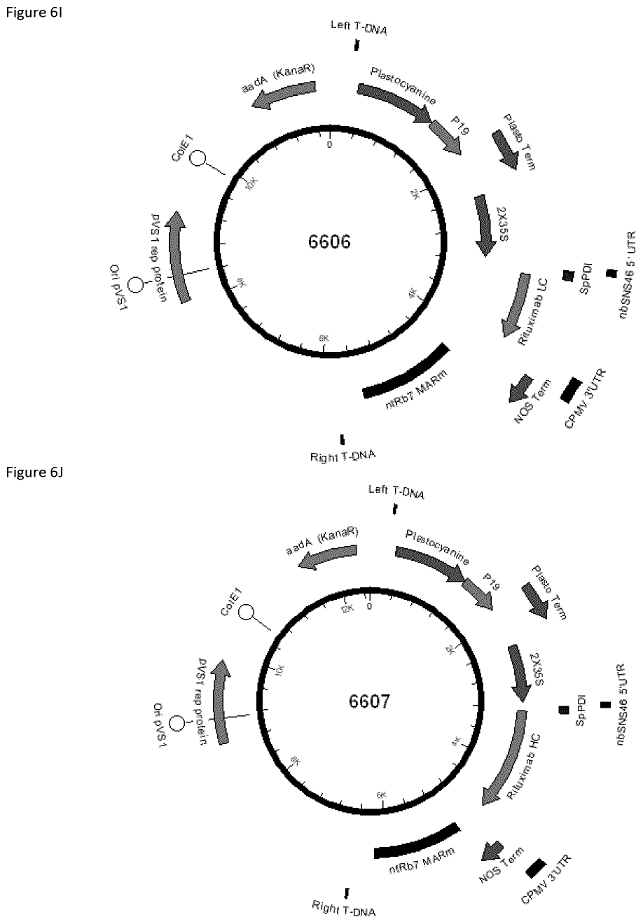

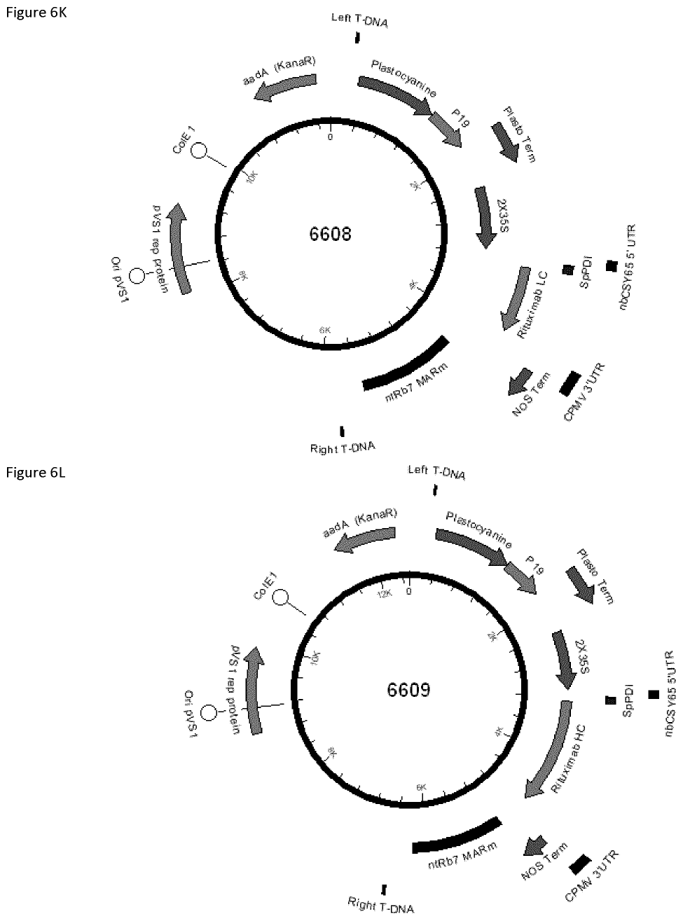

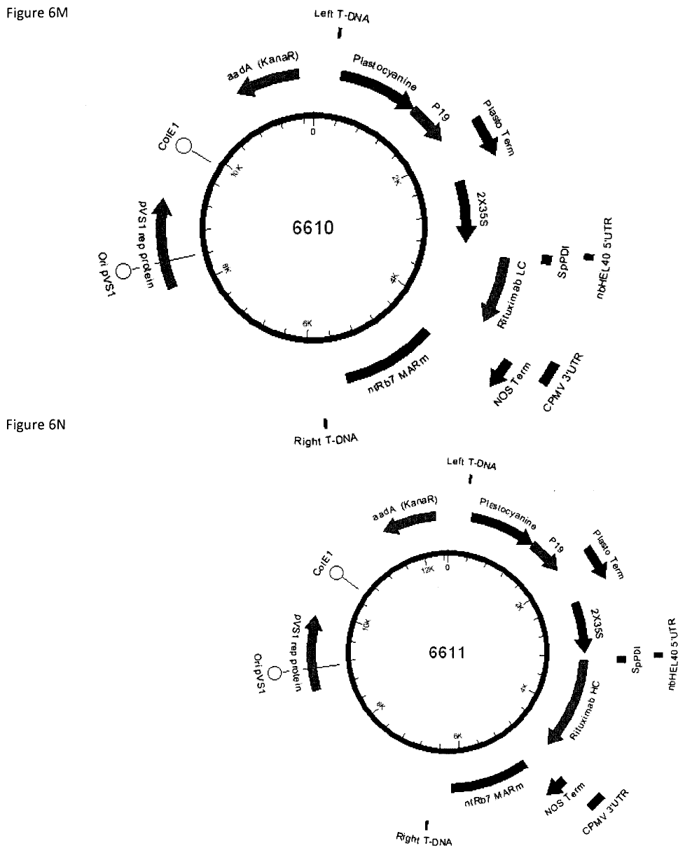

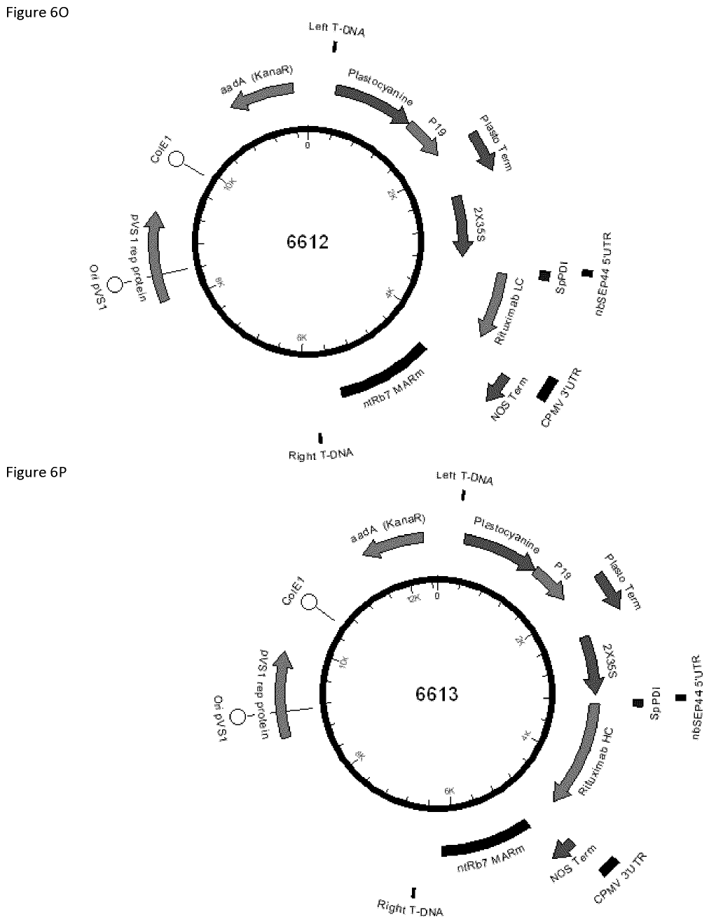

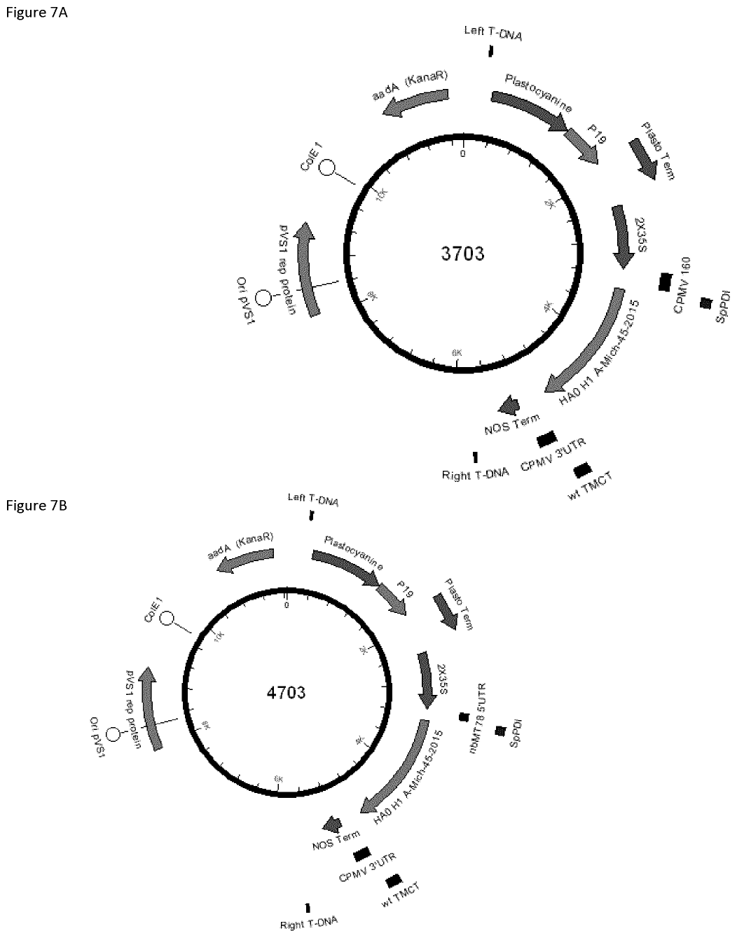

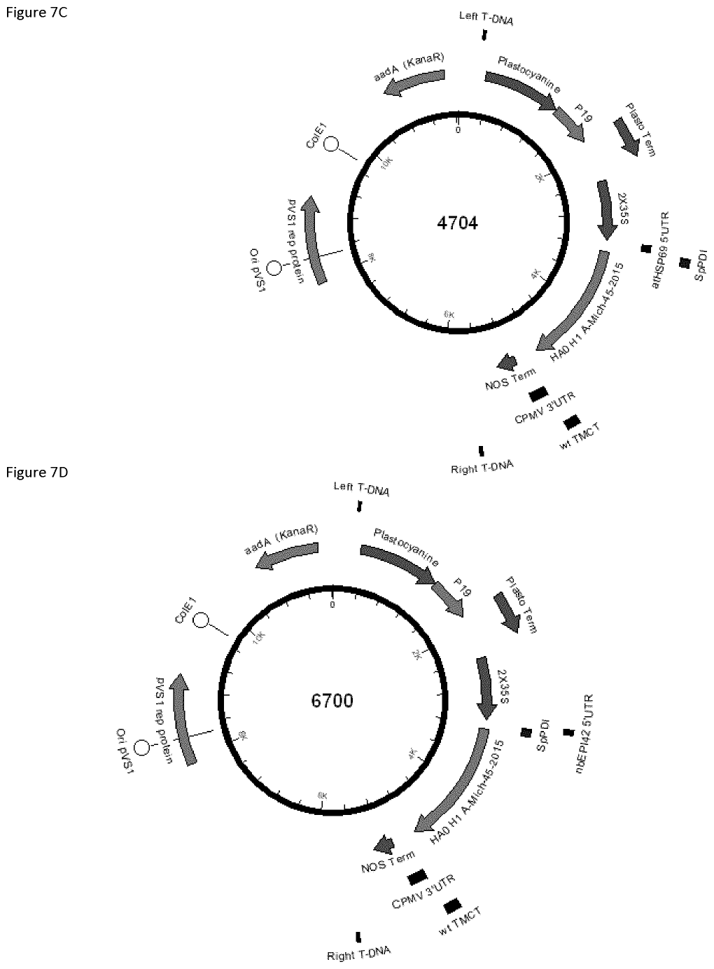

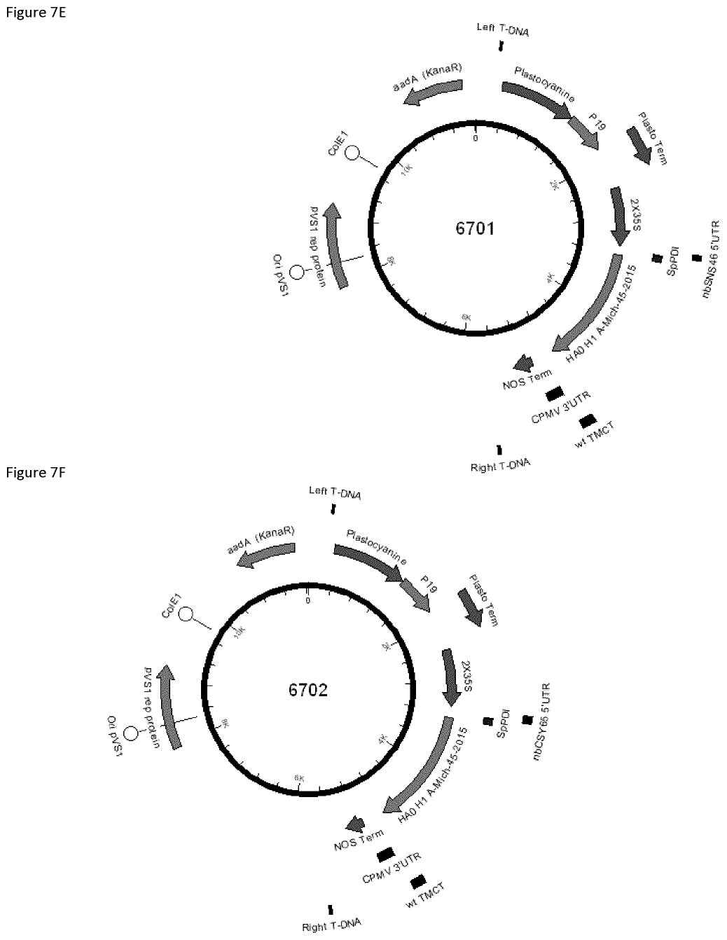

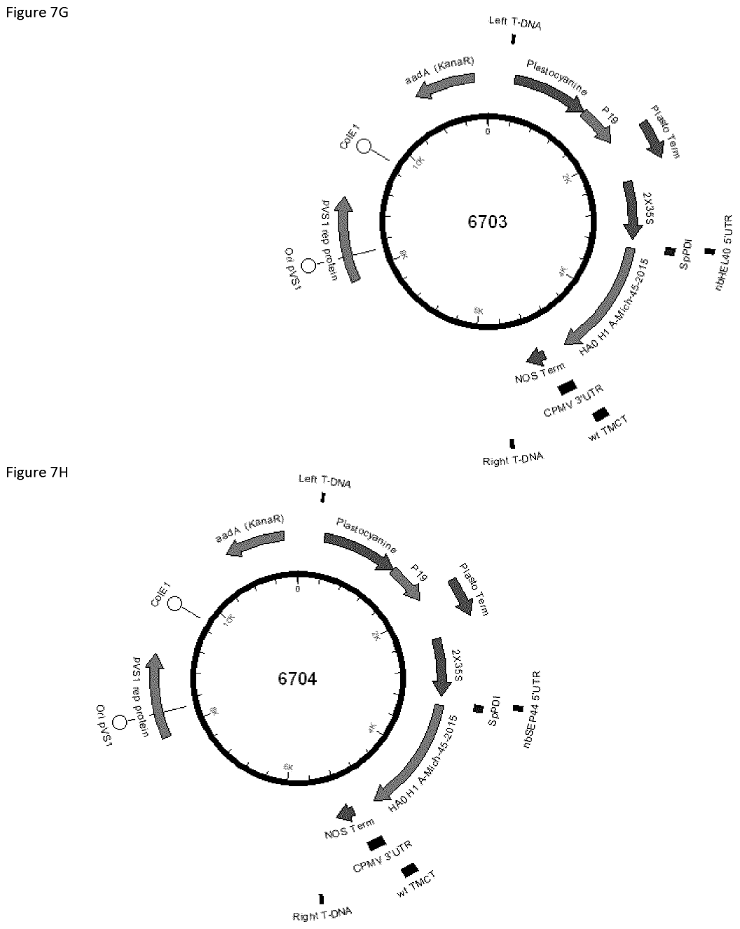

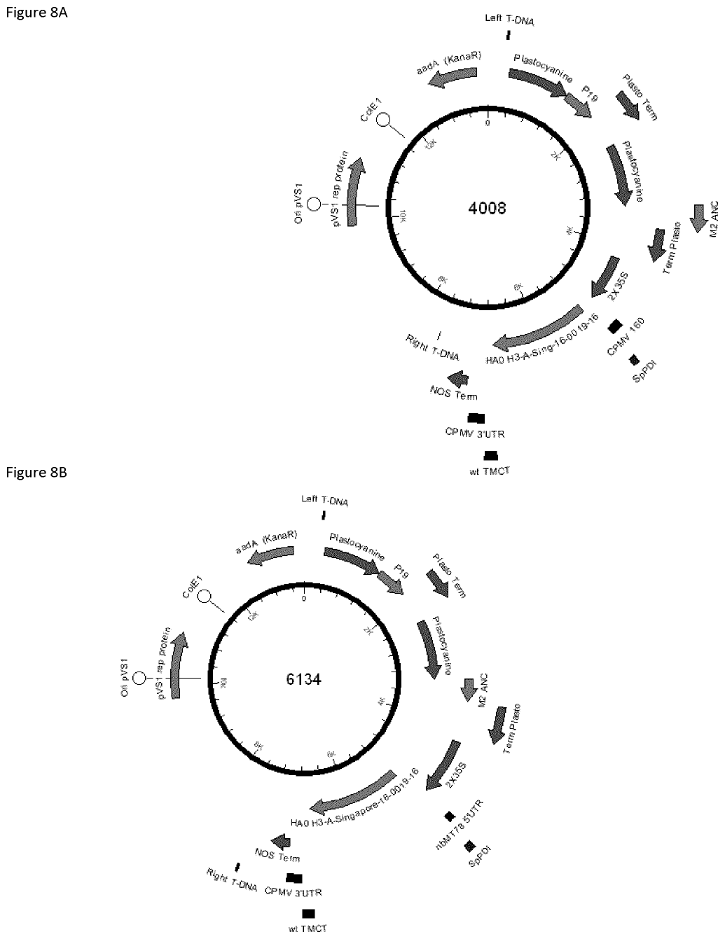

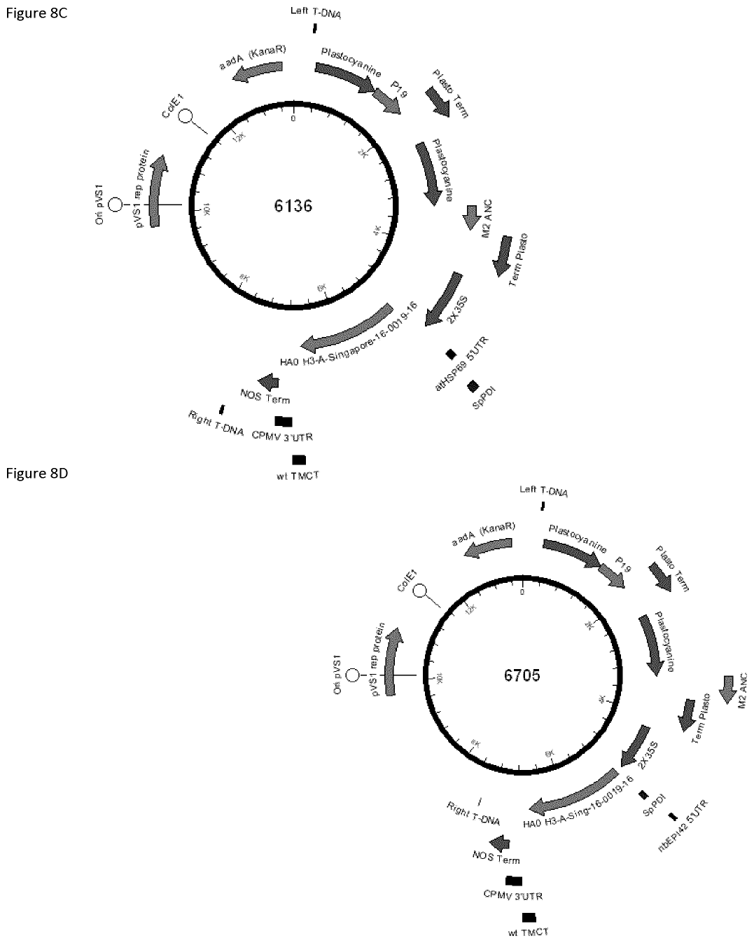

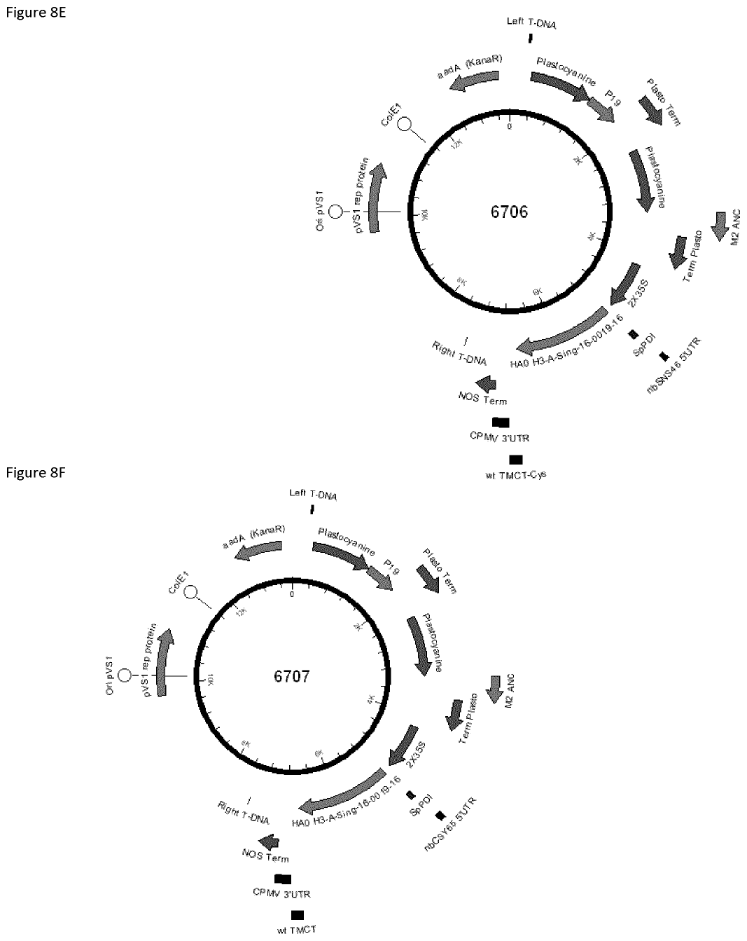

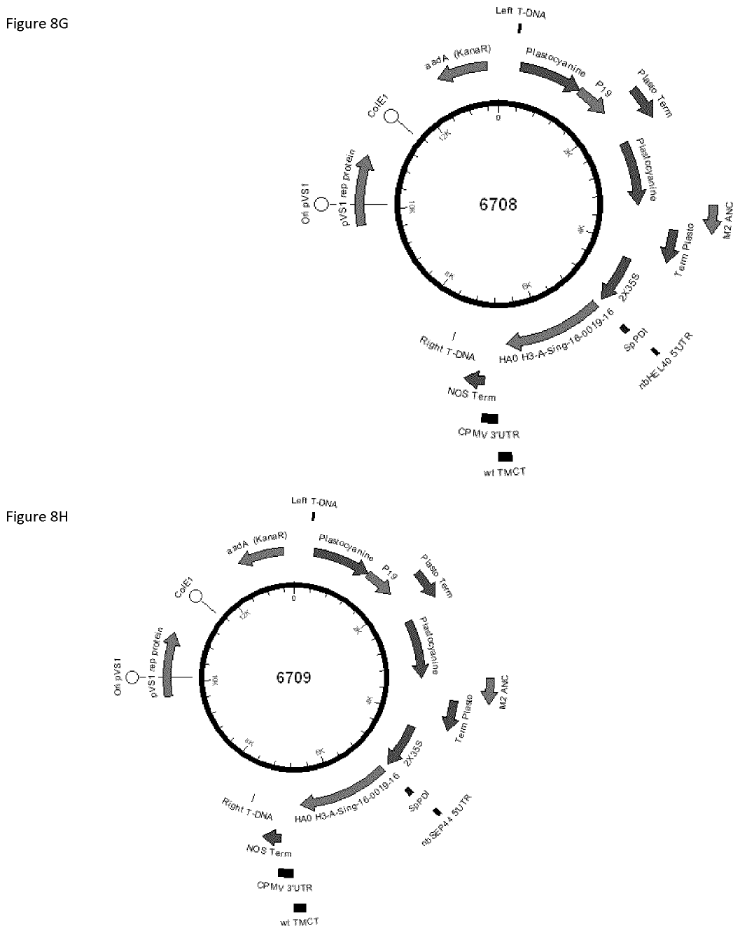

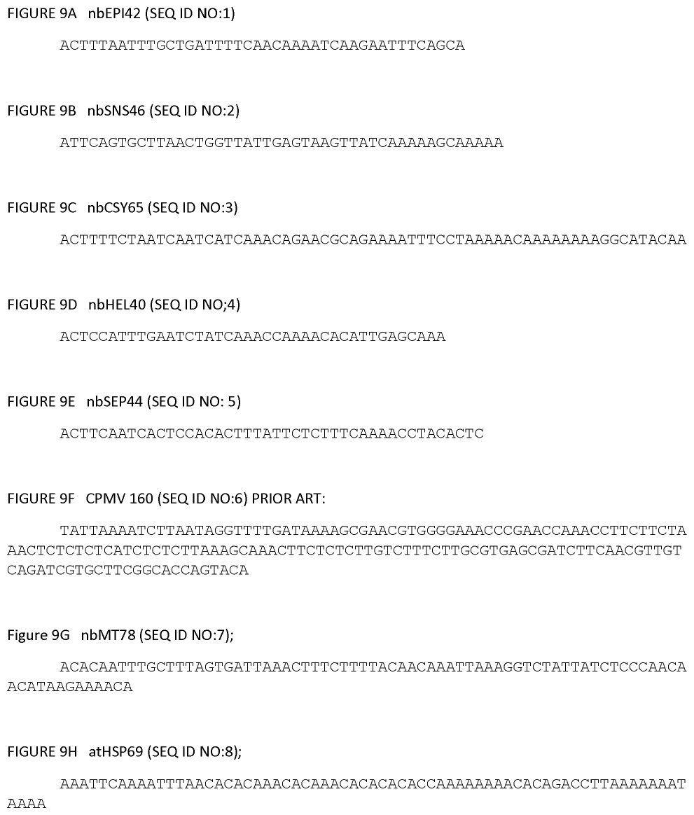

Use of one or more than one of the above expression enhancer of SEQ ID NO's:1-5, was observed to result in the expression of the nucleic acid of interest, or result in an increased expression of a nucleic acid of interest, or a protein of interest (an increase in the yield, the activity, or both the yield and activity, of the protein of interest) as shown with reference to FIGS. 2-5.

With reference to FIGS. 2A, 2B, 3A and 3B, each of the expression enhancers, nbEPI42 (SEQ ID NO:1); nbSNS46 (SEQ ID NO:2); nbCSY65 (SEQ ID NO:3); nbHEL40 (SEQ ID NO:4); and nSEP44 (SEQ ID NO:5) when operatively linked to a nucleic acid sequence encoding a protein of interest, were observed to result in a similar, or an increased, expression or activity of the protein, either Dasher (Dasher GFP; FPOB-27E-269; from ATUM.bio; FIG. 2A), norovirus (GII.4/Syd/12; FIG. 2B), influenza hemagglutinin (H3 A/Sing 19-0019-16, FIG. 3A; and H1 A/Mich/45/15, FIG. 3B;), when compared to the activity of the prior art expression enhancer sequence CMPV 160 (SEQ ID NO:1; WO 2015/103704) operatively linked to the same nucleic acid sequence encoding the same proteins of interest, and expressed under similar conditions, or where indicated, when compared to the activity of the nbMT78 or atHSP69 (both described in U.S. Provisional Application No. 62/643,053, filed Mar. 14, 2018, which is incorporated herein by reference) operatively linked to the same nucleic acid sequence encoding the same proteins of interest, and expressed under similar conditions.

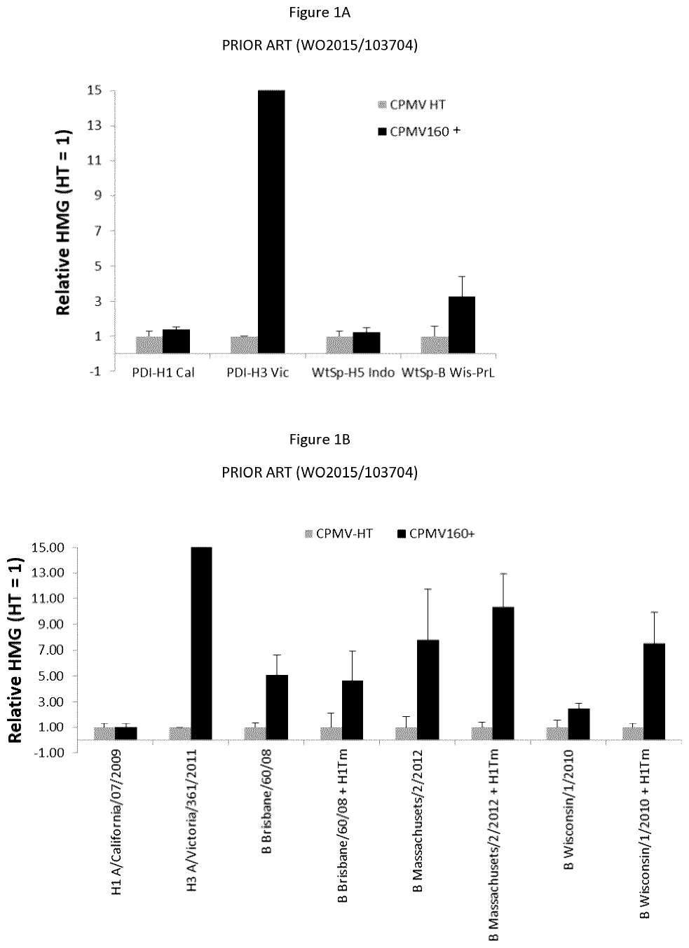

The activity of the prior art expression enhancer CPMV 160 (SEQ ID NO:6), operatively linked to a nucleic acid sequence encoding a protein of interest, is shown relative to the prior art expression enhancer, CPMV-HT, in FIGS. 1A and 1B. The CPMV HT enhancer element refers to a nucleotide sequence encoding the 5′UTR regulating the Cowpea Mosaic Virus (CPMV) RNA2 polypeptide or a modified CPMV sequence as described in WO2009/087391; Sainsbury F., and Lomonossoff G. P., (2008, Plant Physiol. 148; pp. 1212-1218). The CPMV 160 expression enhancer refers to a nucleotide sequence comprising a truncated 5′UTR from CPMV RNA2 as describe in WO 2015/103704. The CPMV 160 and CPMV 160+expression enhancers both comprise the first 160 nucleic acids of the 5′UTR of CMPV RNA 2, however, the CPMV 160+expression enhancer further comprises a multiple cloning site and a plant kozak sequence at the 3′ end of the expression enhancer.

Therefore, the expression enhancers described herein may be used within a plant expression system comprising a regulatory region that is operatively linked with the expression enhancer sequence and a nucleotide sequence of interest.

For example, the present invention provides a method of producing a protein of interest, or increasing production of a protein of interest, for example but not limited to an influenza HA protein, modified influenza HA protein, norovirus protein, or modified norovirus protein, in plants. The method involves introducing a nucleic acid comprising an expression enhancer as described herein operatively linked to nucleotide sequence encoding the protein of interest, for example the influenza HA protein, a modified influenza HA protein, a norovirus protein, or a modified norovirus protein, into the plant, portion of the plant, or plant cell, and expressing the protein in the plant, portion of the plant, or plant cell in a transient or stable manner. Where, the increase in expression may be determined by comparing the level of expression of the nucleotide sequence operatively linked to an expression enhancer, with the level of expression of the same nucleotide sequence that is not operatively linked to the expression enhancer, or for example, when operatively linked to the prior art expression enhancer CPMV 160 (SEQ ID NO:6). Alternatively, the method may comprise providing a plant, portion of the plant, or plant cell that comprises an expression enhancer as described herein operatively linked to nucleotide sequence encoding the protein of interest, for example the influenza HA protein, modified influenza HA protein, norovirus protein, modified norovirus protein, or multimeric protein, and expressing the nucleic acid encoding the protein in the plant, portion of the plant or plant cell in a transient or stable manner.

Furthermore, the present invention provides plant matter, a plant extract, or a protein extract comprising a protein of interest, for example an influenza HA protein, modified influenza HA protein, norovirus protein, or modified norovirus protein. The plant matter, plant extract, or protein extract may be used to induce immunity, for example, to influenza or norovirus infection in a subject. Alternatively, the protein of interest, for example influenza HA protein, modified influenza HA protein, norovirus protein, or modified norovirus protein may be purified or partially purified, and the purified or partially purified preparation may be used to induce immunity, for example, to influenza or norovirus infection in a subject, or the protein of interest, for example the influenza HA protein, modified influenza HA protein, norovirus protein, or modified norovirus protein may be used within a composition for inducing an immune response, and a pharmaceutically acceptable carrier, adjuvant, vehicle, or excipient.

The expression enhancers described herein may be used for the production of any protein of interest or for the production of virus like particles (VLPs). For example, with reference to FIG. 2B, and FIGS. 3A and 3B, the expression enhancers described herein are shown to be effective for the production of Norovirus, and influenza, VLPs, respectively.

Therefore, the present invention also provides a method of producing, or increasing production of VLPs comprising an influenza HA protein, modified influenza HA protein, norovirus protein, or modified norovirus protein, in plants. For example, the method may involve introducing a nucleic acid comprising an expression enhancer as described herein, for example, one or more than one of SEQ ID NO:1-5, operatively linked to nucleotide sequence encoding an influenza HA protein, a modified influenza HA protein, a norovirus protein, or a modified norovirus protein, into the plant, portion of the plant, or plant cell in a transient or stable manner, and expressing the protein in the plant, portion of the plant, or plant cell, in order to produce a VLP comprising the influenza HA protein, modified influenza HA protein, norovirus protein, or modified norovirus protein. The increase in expression may be determined by comparing the level of expression of the nucleotide sequence operatively linked to an expression enhancer, with the level of expression of the same nucleotide sequence that is not operatively linked to the expression enhancer, or for example, when operatively linked to the prior art expression enhancer CPMV 160 (SEQ ID NO:6). Alternatively, the method may comprise providing a plant, portion of the plant, or plant cell that comprises an expression enhancer as described herein operatively linked to nucleotide sequence encoding the influenza HA protein, modified influenza HA protein, norovirus protein, or modified norovirus protein, and expressing the nucleic acid encoding the protein in order to produce a VLP comprising the influenza HA protein, modified influenza HA protein, norovirus protein, or modified norovirus protein.

Furthermore, the present invention provides plant matter, a plant extract, or a protein extract comprising VLPs comprising an influenza HA protein, modified influenza HA protein, norovirus protein, or modified norovirus protein. The plant matter, plant extract, or protein extract may be used to induce immunity to norovirus infection in a subject. Alternatively, VLPs comprising the influenza HA protein, modified influenza HA protein, norovirus protein, or modified norovirus protein may be purified or partially purified, and the purified or partially purified preparation may be used to induce immunity to a norovirus infection in a subject, or the VLPs comprising influenza HA protein, modified influenza HA protein, norovirus protein, or modified norovirus protein may be used within a composition for inducing an immune response, and a pharmaceutically acceptable carrier, adjuvant, vehicle, or excipient.

The expression enhancers described herein may also be used for the production of a multimeric protein of interest, for example an antibody. As shown with reference to FIG. 3C, co-expression of two nucleic acids encoding the light chain (LC) and the heavy chain (HC) of an antibody, for example Rituximab, may be expressed in a plant, when each of the nucleic acid sequences are operatively linked to the same or different expression enhancers described herein. For example, co-expression of a first nucleic acid, encoding the HC of rituximab, and operatively linked to the expression enhancer nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5), along with a second nucleic acid, encoding the LC of rituximab, and operatively linked to either the same expression enhancer, nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5), respectively, resulted in expression of the multimeric protein, or an increase in expression of the multimeric protein, when compared to co-expression of first and second nucleic acids encoding the same HC and LC sequences, but each of the first and second nucleic acids operatively linked to the prior art expression enhancer CPMV 160 (SEQ ID NO:6). The first and second nucleic acids may also be co-expressed using other combination of the expression enhancers described herein, in a similar manner as described in U.S. Provisional Application No. 62/643,053 (filed Mar. 14, 2018, which is incorporated herein by reference).

Therefore, the present invention also provides a method of producing, or increasing production of a multimeric protein in plants. For example, the method may involve introducing, in a transient or stable manner, a first nucleic acid comprising an expression enhancer as described herein operatively linked to nucleotide sequence encoding a first protein component, and a second nucleic acid comprising an expression enhancer as described herein operatively linked to nucleotide sequence encoding a second protein component, into the plant, portion of the plant, or plant cell, and co-expressing the first and second nucleic acids in the plant, portion of the plant, or plant cell, in order to produce the multimeric protein. The increase in expression may be determined by comparing the level of expression of the first and second nucleic acid, each operatively linked to an expression enhancer(s), with the level of expression of the same first and second nucleic acids, each of which are not operatively linked to the expression enhancer(s), or for example, when operatively linked to the prior art expression enhancer CPMV 160 (SEQ ID NO:6). Alternatively, the method may comprise providing a plant, portion of the plant, or plant cell that comprises the first nucleic acid comprising an expression enhancer as described herein operatively linked to nucleotide sequence encoding a first protein component, and a second nucleic acid comprising an expression enhancer as described herein operatively linked to nucleotide sequence encoding a second protein component and co-expressing the first and second nucleic acid sequences to produce the multimeric protein.

Furthermore, the present invention provides plant matter, a plant extract, or a protein extract comprising the multimeric protein, or the multimeric protein may be purified or partially purified.

As described herein, there is provided a nucleic acid construct comprising an expression enhancer sequence operatively linked to a nucleotide sequence of interest encoding a protein of interest. Also provided are plant expression systems and vectors, comprising the construct or one or more than one nucleic acid comprising an enhancer sequence as described herein. Also provided is a plant expression system, a vector, a construct, or a nucleic acid, comprising a plant regulatory region, in operative association with an enhancer sequence that is operatively linked to a nucleotide sequence of interest, the nucleotide sequence of interest encoding a protein of interest. The enhancer sequence may be selected from any one of SEQ ID NO's:1, 5, or a nucleotide sequence that exhibits 100%, 99%, 98%, 97%, 96%, 95%, or 90%, or any amount therebetween, sequence identity to the sequence as set forth in any one of SEQ ID NO's:1-5, wherein, the expression enhancer, when operatively linked to a nucleic acid of interest, results in the expression of the nucleic acid of interest, or increases the level of expression of the nucleic acid of interest when compared to the level of expression of the same nucleic acid of interest that is not operatively linked to the expression enhancer, or for example, when operatively linked to the prior art expression enhancer CPMV 160 (SEQ ID NO:6).

The enhancer sequence of any one of SEQ ID NO's:1-5 may be modified using methods known to one of skill in the art, including deletion, insertion, and/or substitution of one or more than one nucleotide of the enhancer sequence, to produce an expression enhancer that results in a similar or increased enhancer activity, or that results in another beneficial property of the expression enhancer. For example, a beneficial property may include improved transcriptional initiation, improved mRNA stability, improved mRNA translation, or a combination thereof.

The enhancer sequence of the present invention may be used to express a protein of interest in a host organism for example a plant. In this case, the protein of interest may also be heterologous to the host organism in question and introduced into the plant cells using transformation techniques know in the art. A heterologous gene in an organism may replace an endogenous equivalent gene, i.e. one which normally performs the same or a similar function, or the inserted sequence may be additional to the endogenous gene or other sequence.

The invention further provides an expression cassette comprising in series, a promoter or plant regulatory region, operatively linked to an expression enhancer sequence as described herein which is fused with a nucleotide sequence of interest, a 3′UTR sequence, and a terminator sequence. The enhancer sequence may be defined by, any one of SEQ ID NO's:1-5, or a nucleotide sequence that exhibits 100%, 99%, 98%, 97%, 96%, 95%, or 90%, sequence identity, or any amount therebetween, to the sequence as set forth in any one of SEQ ID NO's:1-5. The enhancer sequence may also be modified using techniques known to one of skill in the art, provided that the enhancer sequence results in the expression of the nucleic acid of interest, or increases the level of expression of the nucleotide sequence of interest, for example, determined by comparing the level of expression of the nucleotide sequence operatively linked to an expression enhancer, with the level of expression of the same nucleotide sequence that is not operatively linked to the expression enhancer. or for example. when operatively linked to the prior art expression enhancer CPMV 160 (SEQ ID NO:6).

The sequences described in the present application are listed in Table 1.

TABLE 1
List of nucleic acid and amino acid sequences:

| | SEQ ID | |
|---|---|---|
| | NO: | Fig # |
| nbEPI42 | 1 | 9A |
| nbSNS46 | 2 | 9B |
| nbCSY65 | 3 | 9C |
| nbHEL40 | 4 | 9D |
| nbSEP44 | 5 | 9E |
| CPMV 160 | 6 | 9F |
| nbMT78 | 7 | 9G |
| atHSP69 | 8 | 9H |
| IF-(2X35S + C)_CPMV160.c | 9 | 10A |
| IF-Dasher(27-609).r | 10 | 10B |
| CPMV 160 5′UTR-Dasher | 11 | 10C |
| Dasher (na) | 12 | 10D |
| Dasher (aa) | 13 | 10E |
| IF-nbMT78.c | 14 | 10F |
| nbMT78_Dasher.c | 15 | 10G |
| atHSP69_Dasher.c | 16 | 10H |
| IF-atHSP69.c | 17 | 10I |
| nbEPI42 + Dasher.c | 18 | 10J |
| IF-nbEPI42.c | 19 | 10K |
| nbSNS46 + Dasher.c | 20 | 10L |
| IF-nbSNS46.c | 21 | 10M |
| nbCSY65 + Dasher.c | 22 | 10N |
| IF-nbCSY65.c | 23 | 10O |
| nbHEL40 + Dasher.c | 24 | 10P |
| IF-nbHEL40.c | 25 | 10Q |
| nbSEP44 + Dasher.c | 26 | 10R |
| IF-nbSEP44.c | 27 | 10S |
| IF-GII4Syd12VP1.r | 28 | 11A |
| CPMV 160 5′UTR-VP1 | 29 | 11B |
| (GII.4) | | |
| VP1 (GII.4) (na) | 30 | 11C |
| VP1 (GII.4) (aa) | 31 | 11D |
| nbMT78 + GII4Syd12.c | 32 | 11E |
| atHSP69 + GII4Syd12.c | 33 | 11F |
| nbEPI42 + GII4Syd12.c | 34 | 11G |
| nbSNS46 + GII4Syd12.c | 35 | 11H |
| nbCSY65 + GII4Syd12.c | 36 | 11I |
| nbHEL40 + GII4Syd12.c | 37 | 11J |
| nbSEP44 + GII4Syd12.c | 38 | 11K |
| IF**-HC(Ritux).s1-6r | 39 | 12A |
| CPMV 160 5′UTR- | 40 | 12B |
| PDI + Rituximab HC | | |
| PDI + Rituximab HC (na) | 41 | 12C |
| PDI + Rituximab HC (aa) | 42 | 12D |
| IF**-LC(Ritux).s1-6r | 43 | 12E |
| CPMV 160 5′UTR- | 44 | 12F |
| PDI + Rituximab LC (na) | | |
| PDI + Rituximab LC (na) | 45 | 12G |
| PDI + Rituximab LC (aa) | 46 | 12H |
| nbMT78_SpPDI.c | 47 | 12I |
| atHSP69_SpPDI.c | 48 | 12J |
| nbEPI42 + PDI.c | 49 | 12K |
| nbSNS46 + PDI.c | 50 | 12L |
| nbCSY65 + PDI.c | 51 | 12M |
| nbHEL40 + PDI.c | 52 | 12N |
| nbSEP44 + PDI.c | 53 | 12O |
| IF-H1cTMCT.s1-4r | 54 | 13A |
| CPMV 160 5′UTR- | 55 | 13B |
| PDI + H1 Mich (na) | | |
| PDI + H1 Mich (na) | 56 | 13C |
| PDI + H1 Mich (aa) | 57 | 13D |
| IF-H3Minn15.r | 58 | 14A |
| CPMV 160 5′UTR- | 59 | 14B |
| PDI + H3 sing (na) | | |
| PDI + H3 sing (na) | 60 | 14C |
| PDI + H3 sing (aa) | 61 | 14D |
| Cloning vector 1666 | 62 | 15A |
| Construct 4467 | 63 | 15B |
| Cloning vector 4160 | 64 | 15C |
| Cloning vector 4170 | 65 | 15D |

The present invention will be further illustrated in the following examples.

### Example 1

### Constructs

The following constructs comprising the enhancers identified above were prepared as follows:

2X35S/nbMT78 5′UTR/Dasher/CPMV 3′UTR/NOS term (Construct number 4467; SEQ ID NO:75)

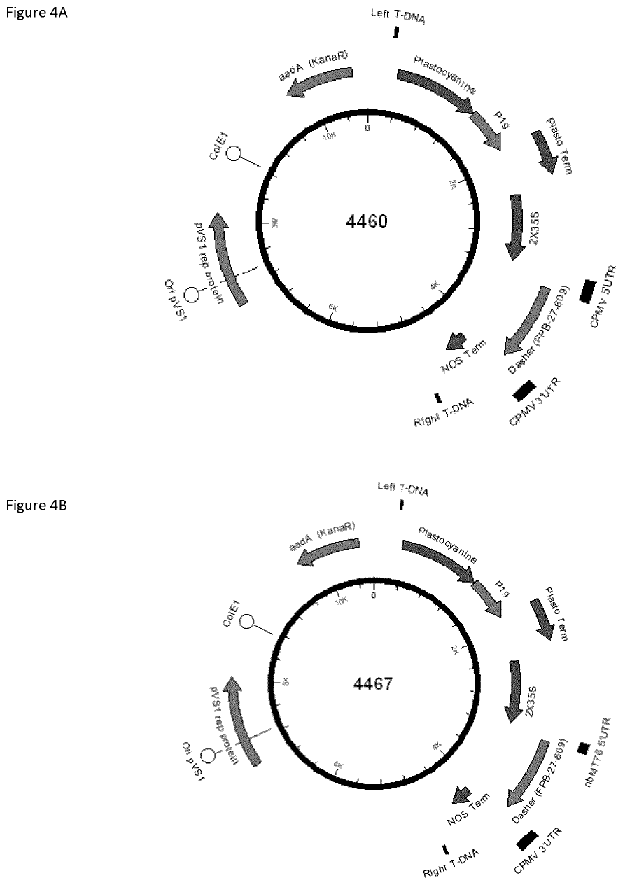

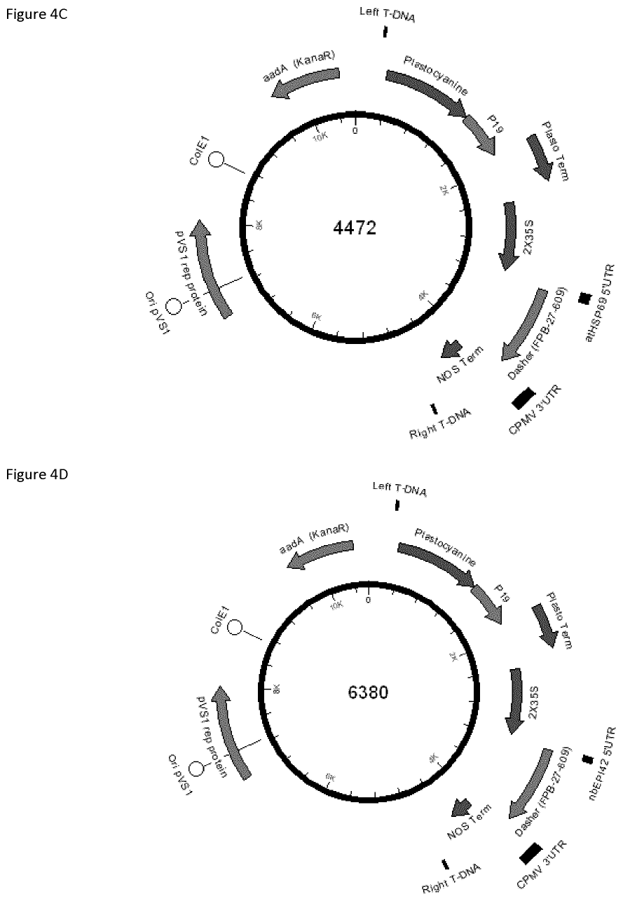

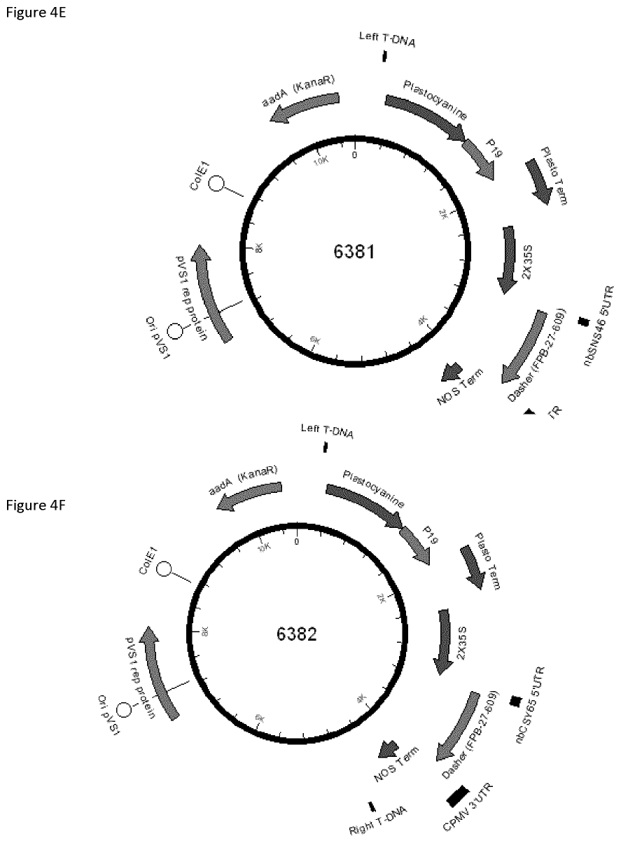

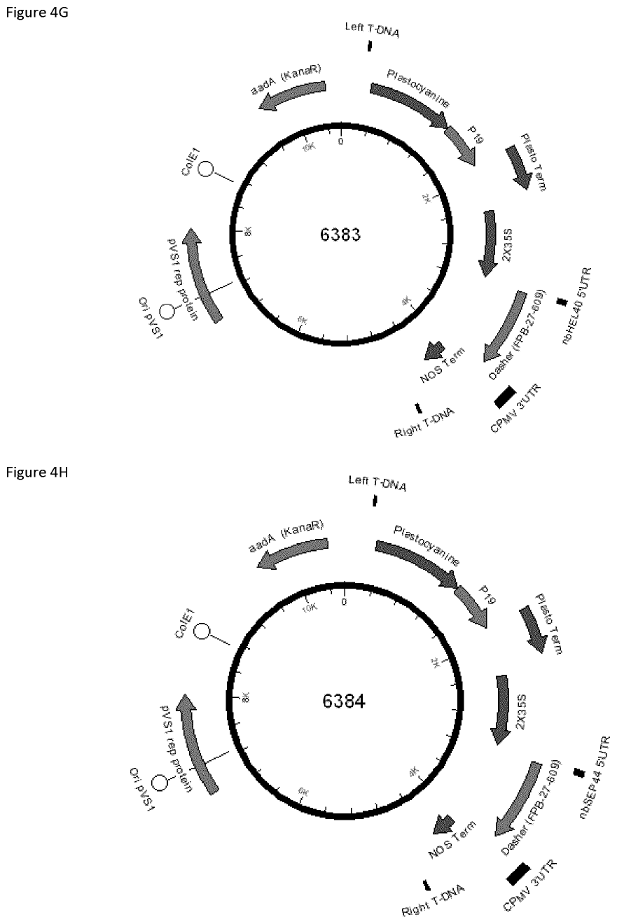

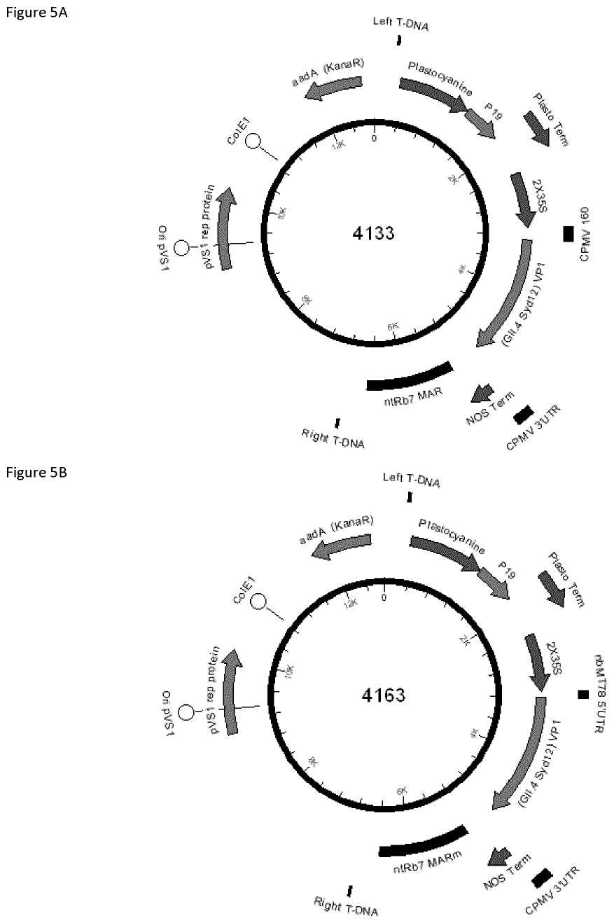

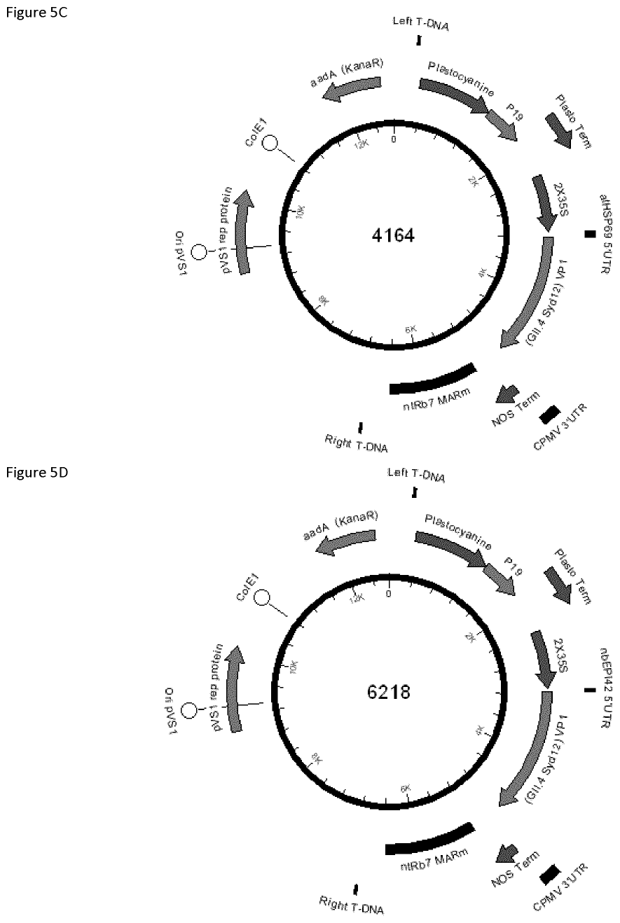

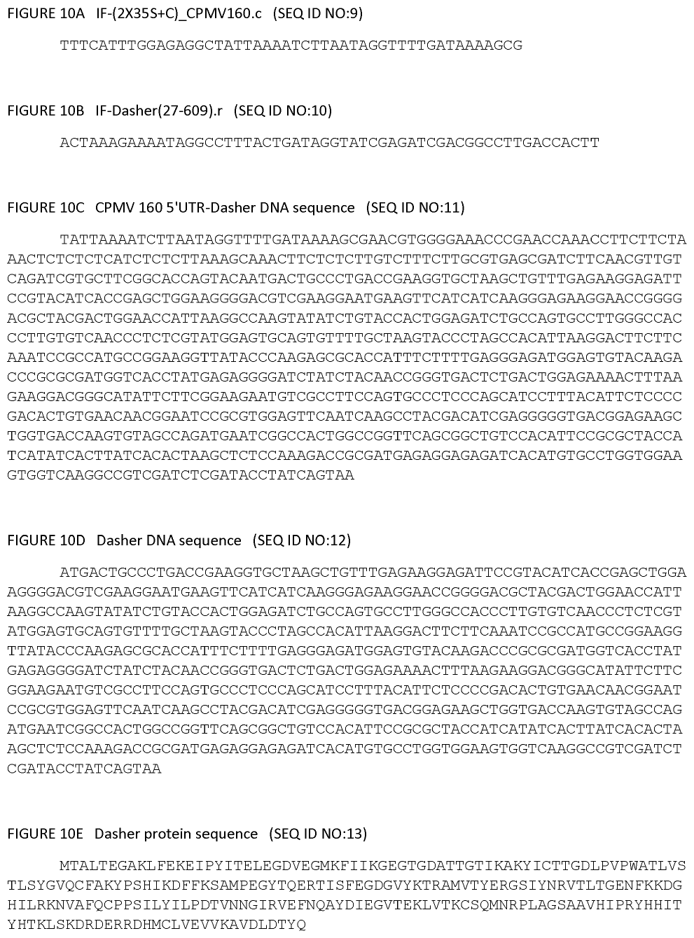

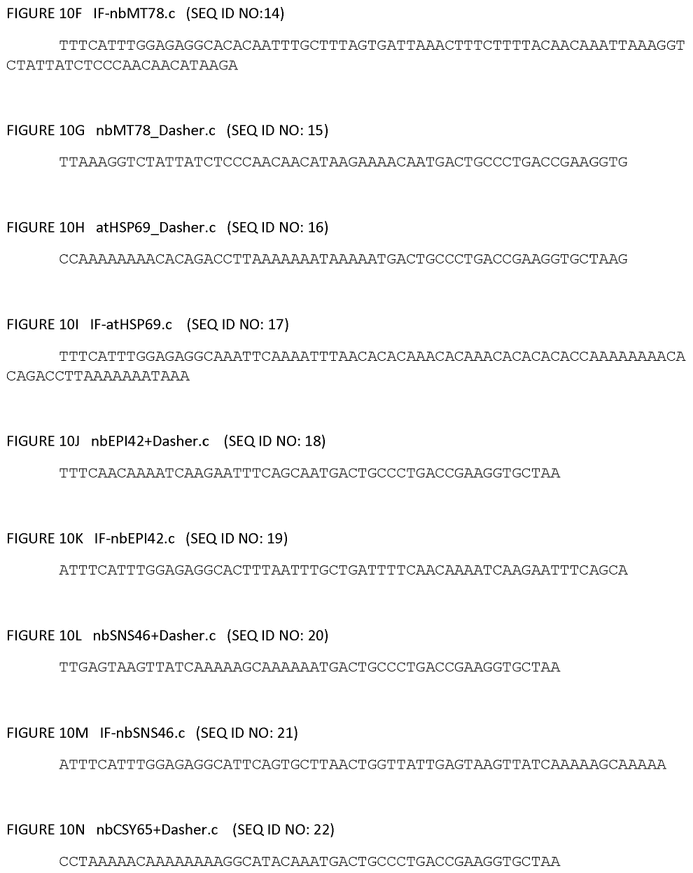

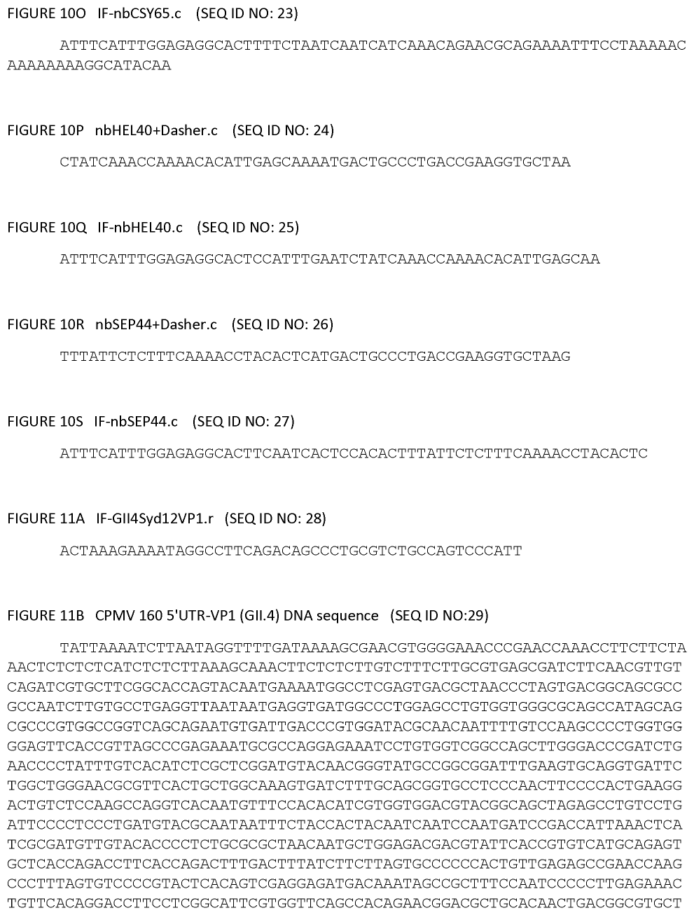

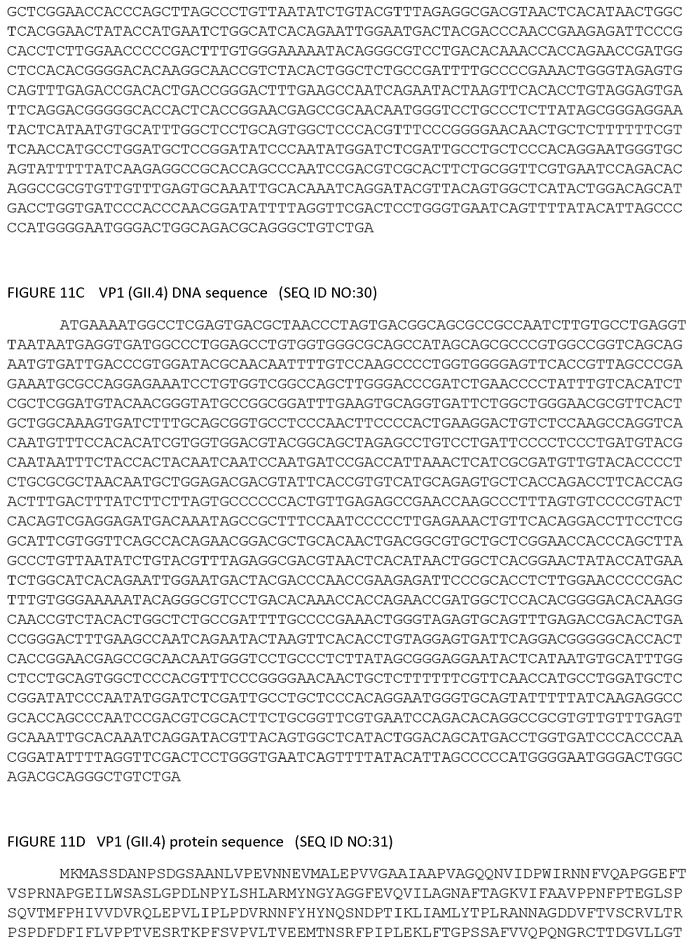

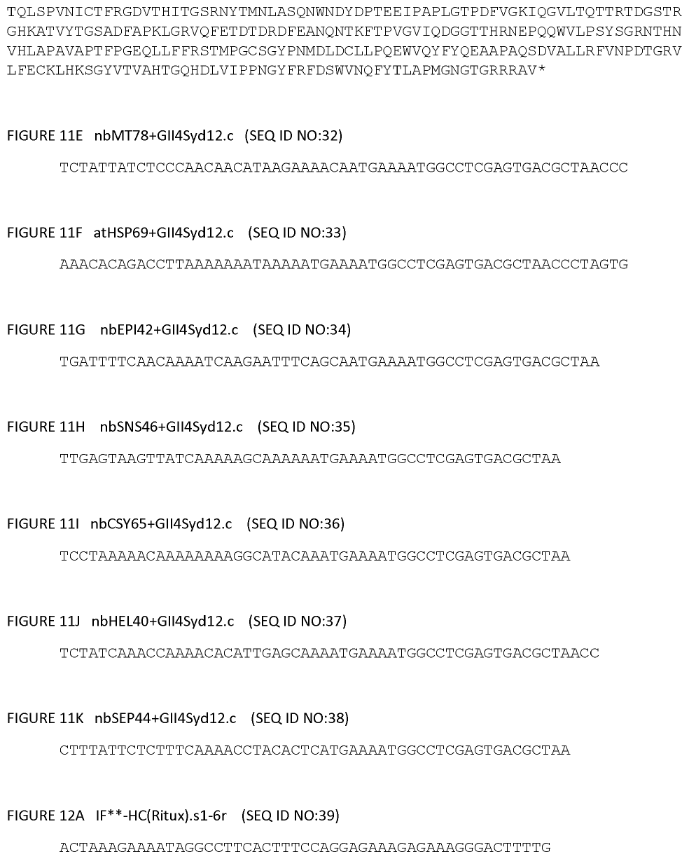

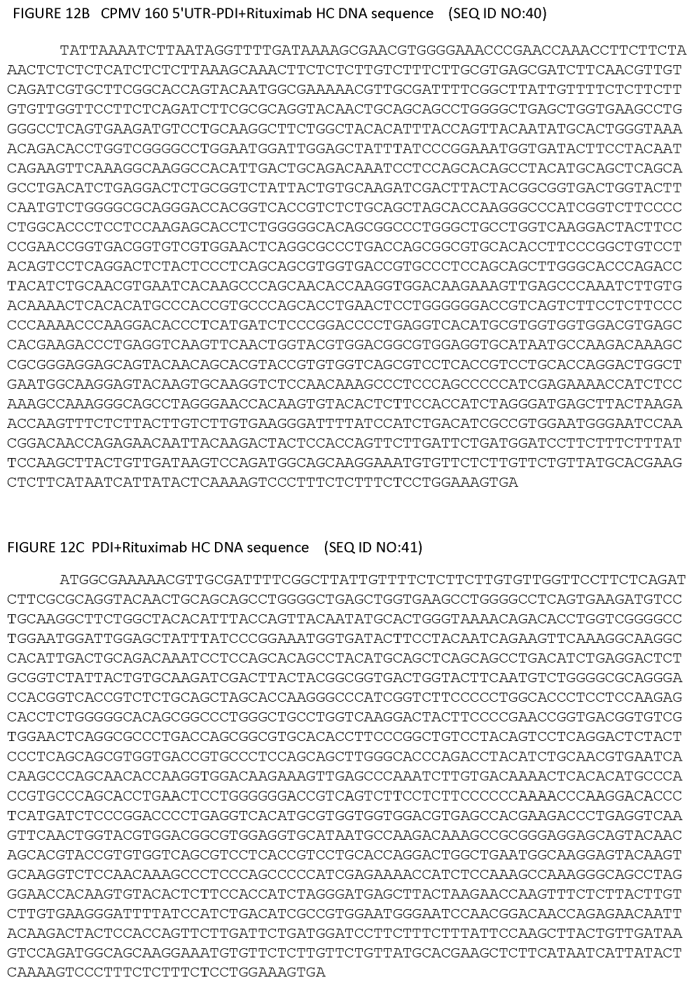

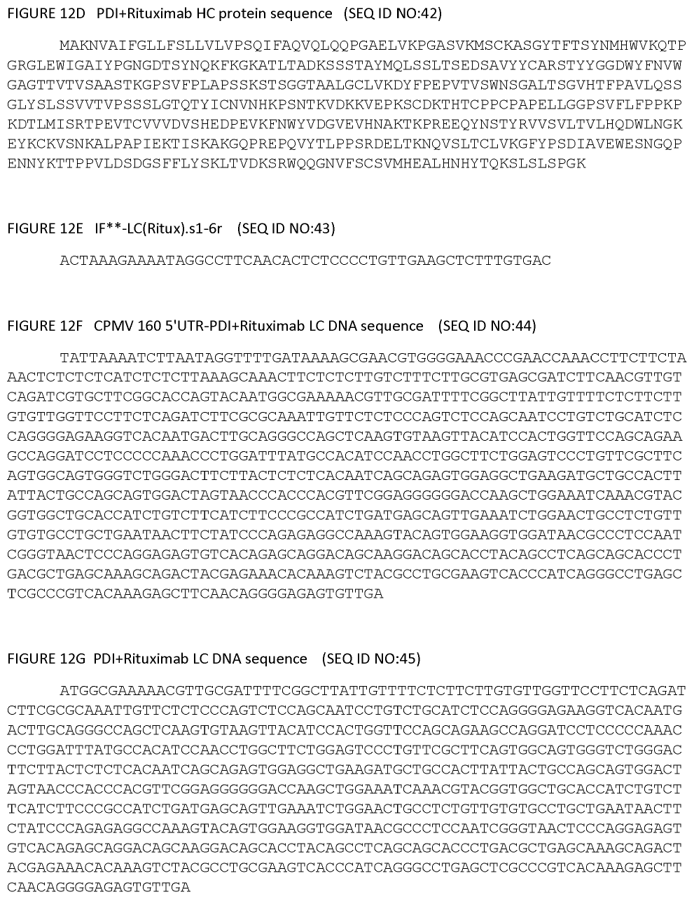

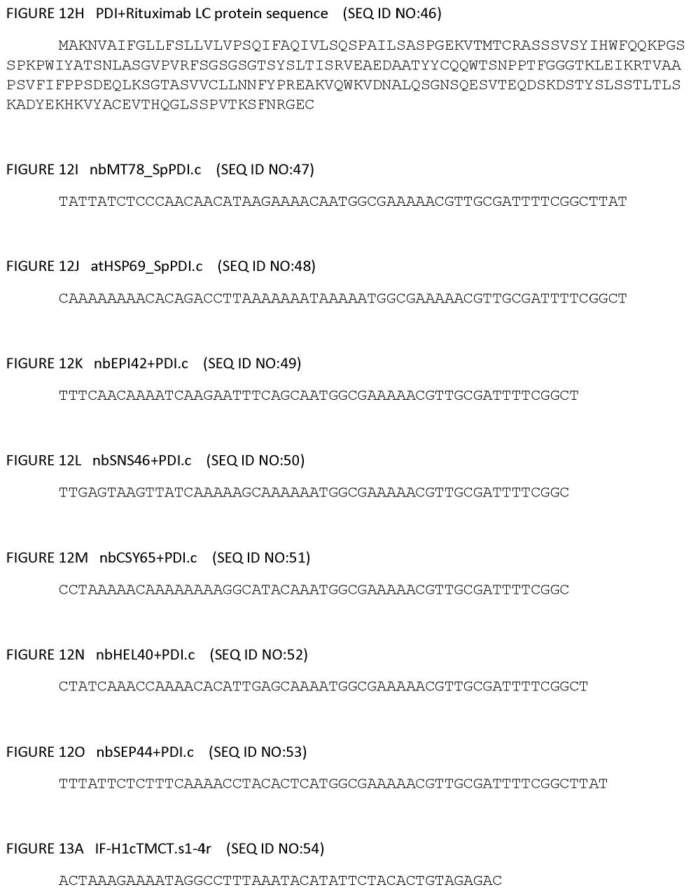

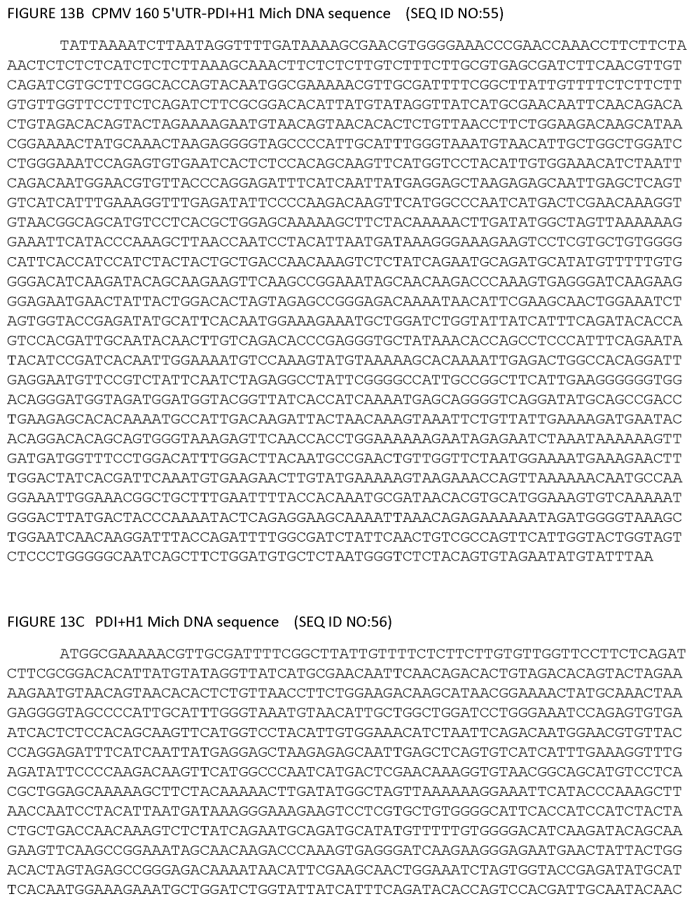

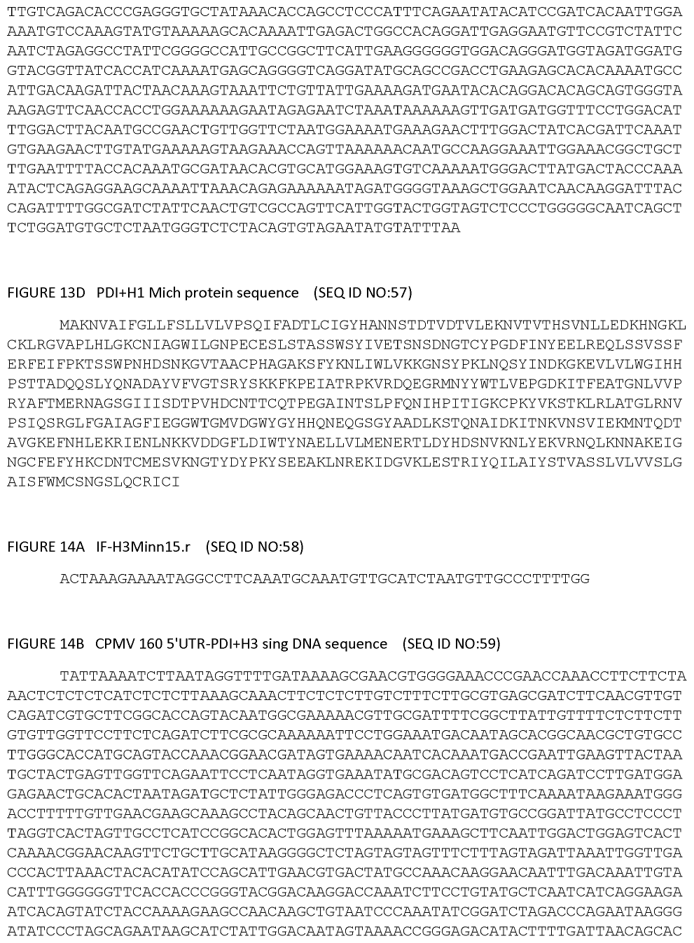

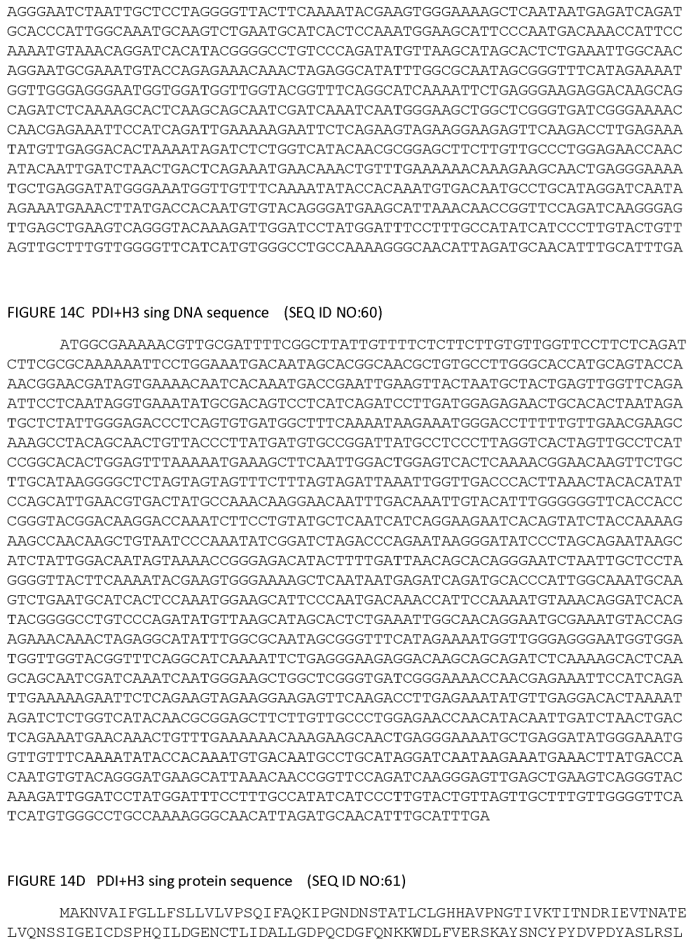

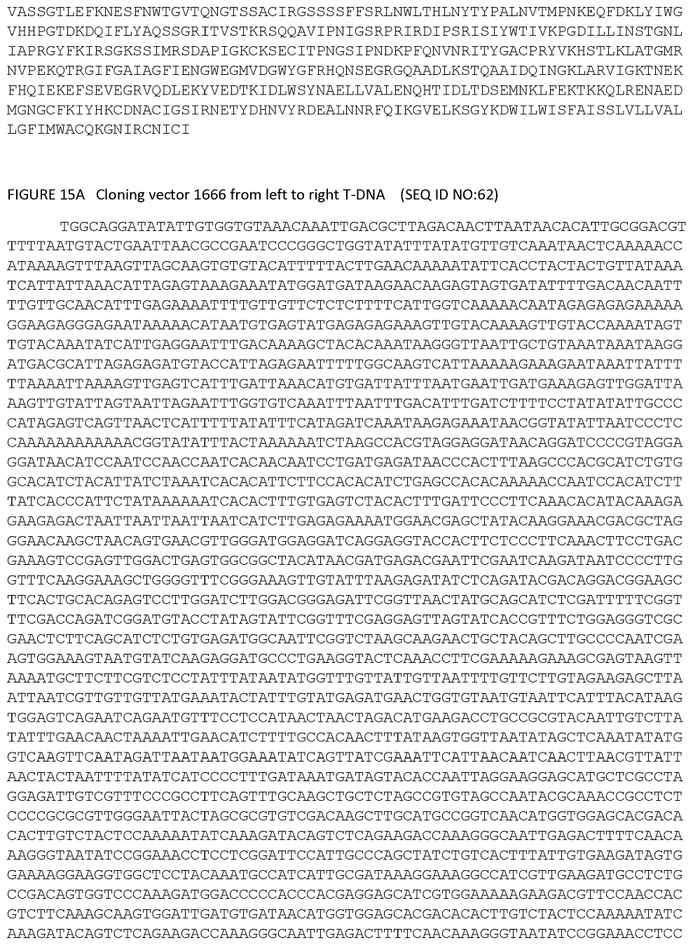

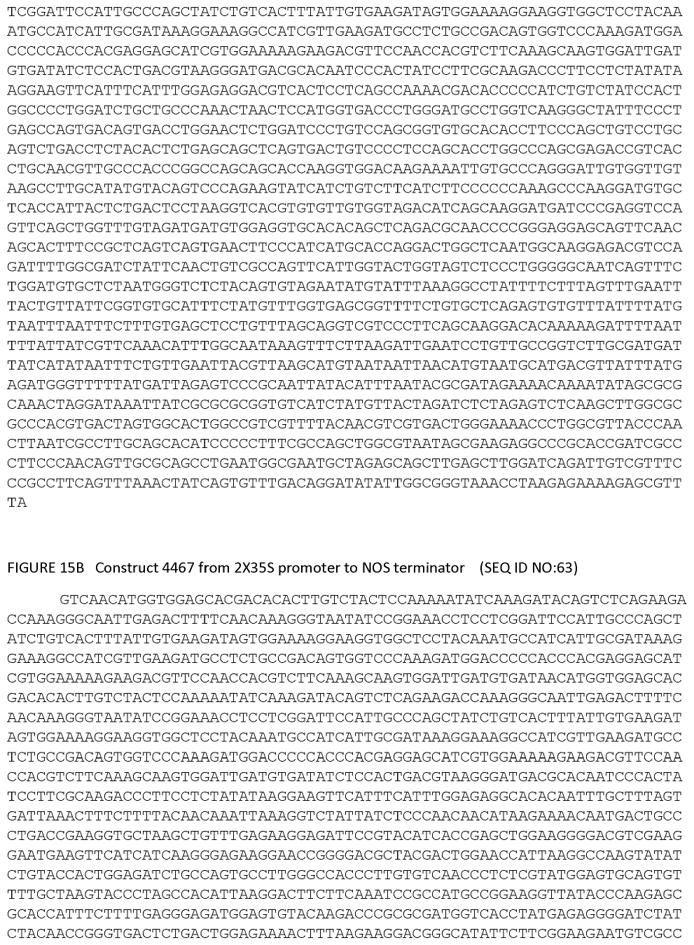

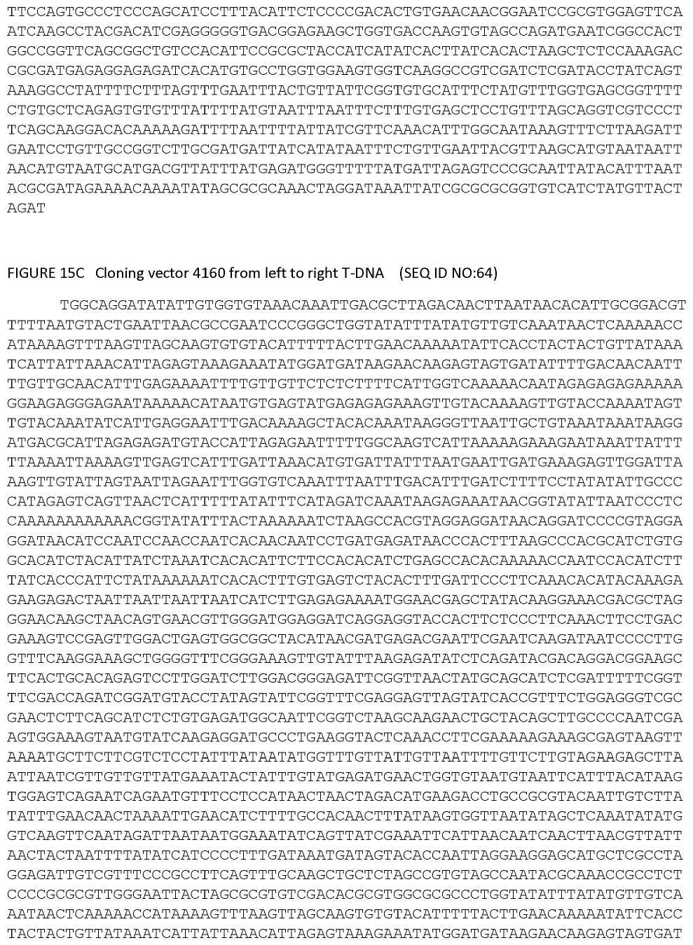

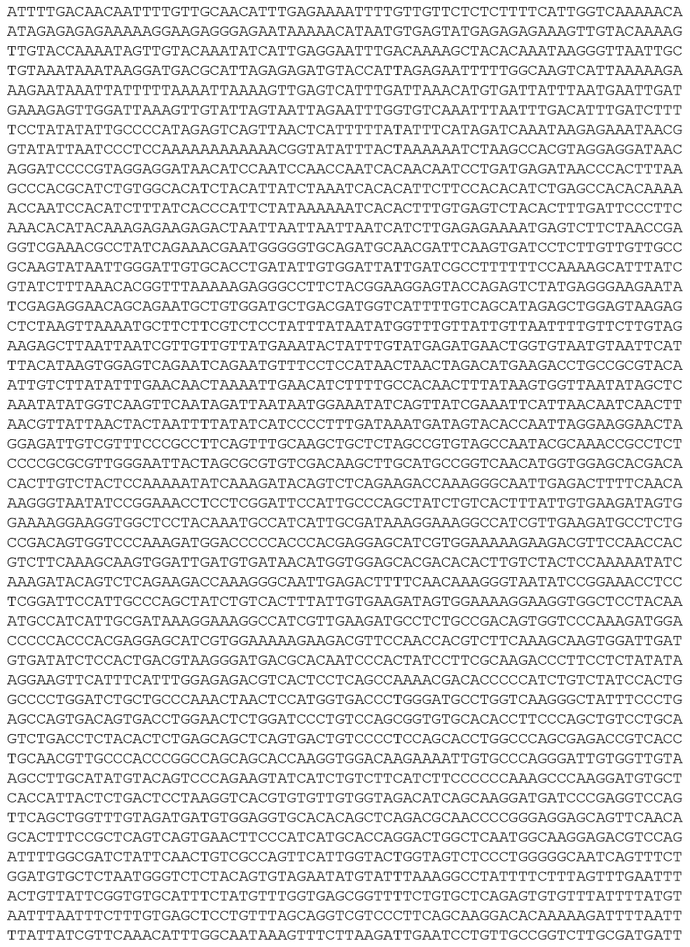

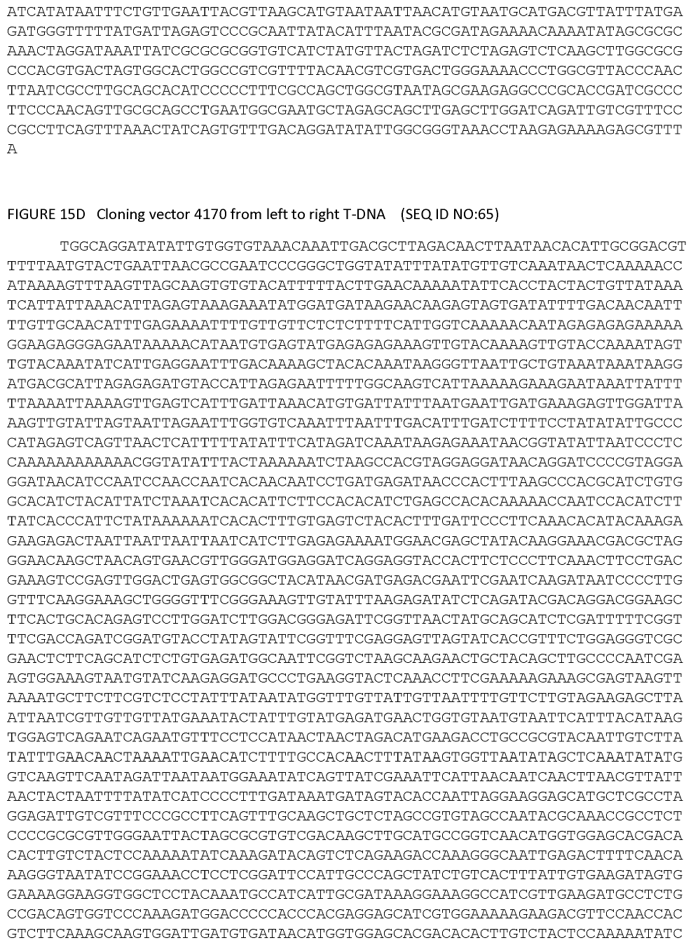

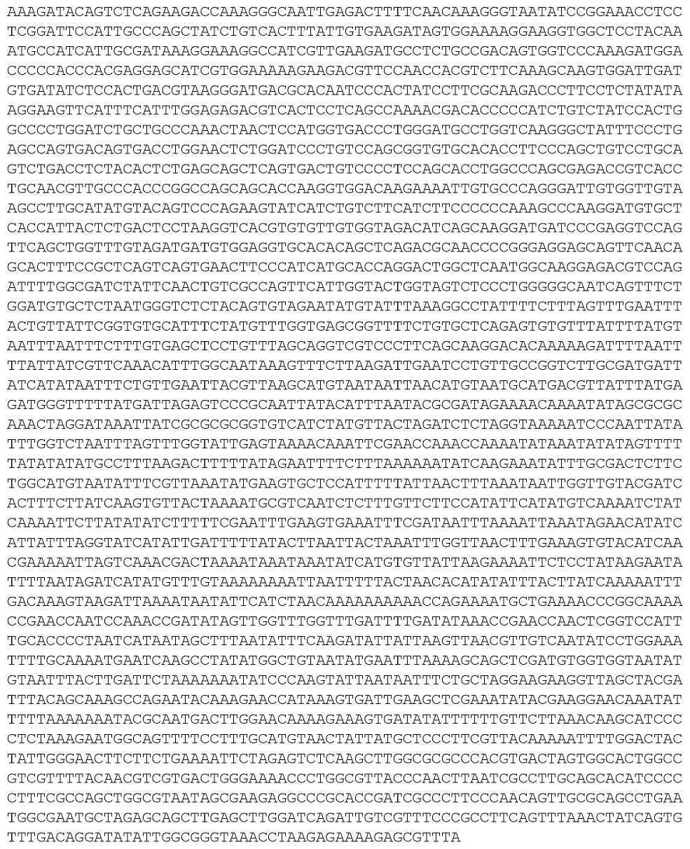

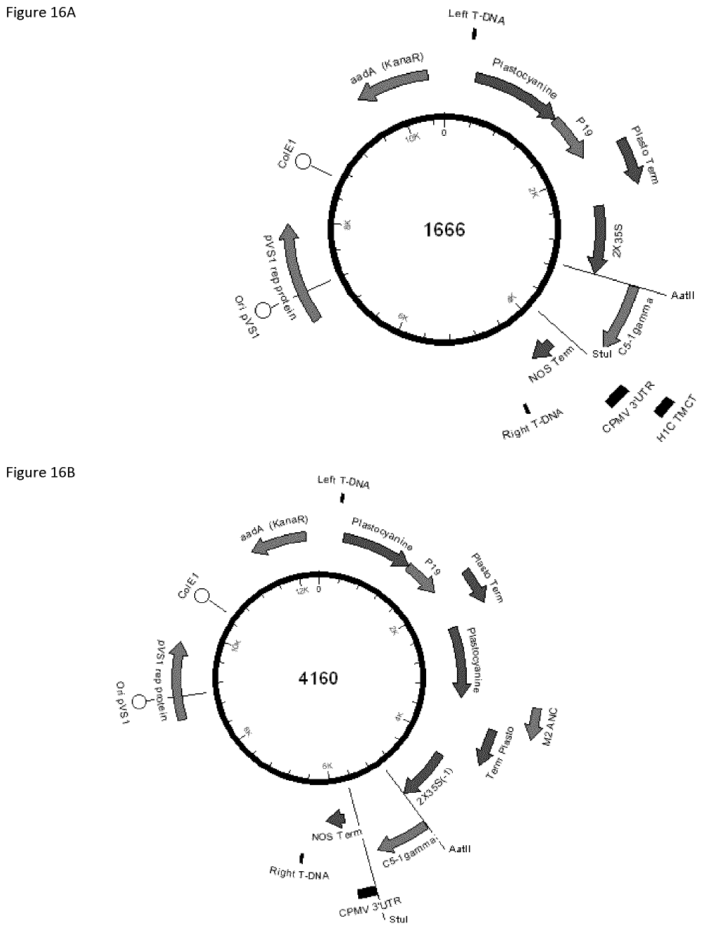

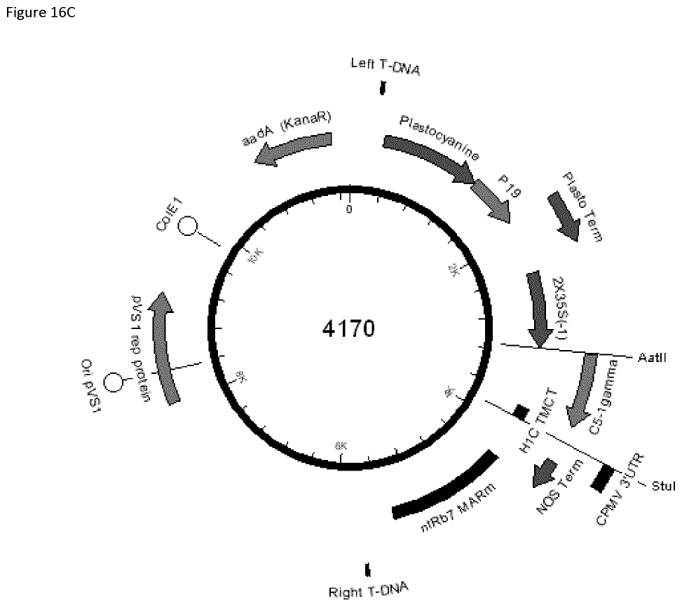

A sequence encoding Dasher fluorescent protein (Atum, Cat #FPB-27-609) fused to the nbMT78 5′UTR was cloned into 2X35S promoter+CPMV 3′UTR/NOS expression system using the following PCR-based method. In a first round of PCR, a fragment containing the Dasher fluorescent protein was amplified using primers nbMT78_Dasher.c (SEQ ID NO:15) and IF-Dasher (27-609).r (SEQ ID NO:10) using Dasher gene sequence (SEQ ID NO:11; FIG. 10C) as template. The PCR product from the first round of amplification (F1 in Table 2) was as used as template to add the atMT78 5′UTR sequence using IF-nbMT78.c (SEQ ID NO:14) and IF-Dasher (27-609).r (SEQ ID NO:10) as primers. The final PCR product (F2 in Table 2) was cloned in 2X35S promoter+CPMV 3′UTR/NOS expression system using In-Fusion cloning system (Clontech, Mountain View, CA). Construct number 1666 (FIG. 16A) was digested with AatII and StuI restriction enzymes and the linearized plasmid was used for the In-Fusion assembly reaction. Construct number 1666 is an acceptor plasmid intended for “In Fusion” cloning of genes of interest in a 2X35S promoter+CPMV 3′UTR/NOS-based expression cassette. It also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO:62 (FIG. 15A). The resulting construct was given number 4467 (SEQ ID NO:63; FIG. 15B). The amino acid sequence of Dasher fluorescent protein is presented in SEQ ID NO:13; FIG. 10E. A representation of construct 4467 is presented in FIG. 4B.

Primers, templates as well as nucleic acid and protein sequences for all constructs described herein, are presented in Table 2.

For influenza H1 construct, the same cloning approach described above for the Dasher fluorescent protein was used.

For Influenza H3 constructs, the cloning vector used, integrates an influenza M2 ion channel gene under the control of Alfalfa Plastocyanin promoter and terminator in addition to the 2X35S promoter+CPMV 3′UTR/NOS-based expression cassette. Plasmid number 4160 (SEQ ID NO:64; FIG. 15C) was digested with AatII and StuI restriction enzymes and used for the In-Fusion reaction in a similar cloning approach as described above for the Dasher fluorescent protein.

For Norovirus VP1 and Rituximab HC and LC constructs, the cloning vector integrates a Matrix attachment region (MAR) regulatory element from the tobacco RB7 gene after the NOS terminator in addition to the 2X35S promoter+CPMV 3′UTR/NOS-based expression cassette. Plasmid number 4170 (SEQ ID NO: 65; FIGS. 15D) was digested with AatII and StuI restriction enzymes and used for the In-Fusion reaction.

TABLE 2
Primers, templates and sequences of interest (SOI) for construct preparation

| | | | | SEQ ID NO: | | | | | | |
|---|---|---|---|---|---|---|---|---|---|---|
| Constructs | | | | | | | F1 | F2 | | |
| 5′UT R | Sp | SOI* | Const # | Primer 1* | Primer 2* | Primer 3* | Template | Template | SOI NA: | SOI AA |
| CPMV 160 | — | Dasher | 4460 | 9 | — | 10 | — | 11 | 12 | 13 |
| nbMT78 | — | Dasher | 4467 | 14 | 15 | 10 | 11 | F1 | 12 | 13 |
| atHSP69 | — | Dasher | 4472 | 17 | 16 | 10 | 11 | F1 | 12 | 13 |
| nbEPI42 | ′ | Dasher | 6380 | 19 | 18 | 10 | 11 | F1 | 12 | 13 |
| nbSNS46 | — | Dasher | 6381 | 21 | 20 | 10 | 11 | F1 | 12 | 13 |
| nbCSY65 | — | Dasher | 6382 | 23 | 22 | 10 | 11 | F1 | 12 | 13 |
| nbHEL40 | | Dasher | 6383 | 25 | 24 | 10 | 11 | F1 | 12 | 13 |
| nbSEP44 | — | Dasher | 6384 | 27 | 26 | 10 | 11 | F1 | 12 | 13 |
| CPMV 160 | | VP1 GII.4 Syd 12 | 4133 | 9 | — | 28 | — | 29 | 30 | 31 |
| nbMT78 | | VP1 GII.4 Syd 12 | 4163 | 14 | 32 | 28 | 29 | F1 | 30 | 31 |
| atHSP69 | | VP1 GII.4 Syd 12 | 4164 | 17 | 33 | 28 | 29 | F1 | 30 | 31 |
| nbEPI42 | | VP1 GII.4 Syd 12 | 6218 | 19 | 34 | 28 | 29 | F1 | 30 | 31 |
| nbSNS46 | | VP1 GII.4 Syd 12 | 6214 | 21 | 35 | 28 | 29 | F1 | 30 | 31 |
| nbCSY65 | | VP1 GII.4 Syd 12 | 6215 | 23 | 36 | 28 | 29 | F1 | 30 | 31 |
| nbHEL40 | | VP1 GII.4 Syd 12 | 6216 | 25 | 37 | 28 | 29 | F1 | 30 | 31 |
| nbSEP44 | | VP1 GII.4 Syd 12 | 6217 | 27 | 38 | 28 | 29 | F1 | 30 | 31 |
| CPMV 160 | PDI | HC Rituximab | 4641 | 9 | — | 39 | — | 40 | 41 | 42 |
| CPMV 160 | PDI | LC Rituximab | 4642 | 9 | — | 43 | — | 44 | 45 | 46 |
| nbMT78 | PDI | LC Rituximab | 6600 | 14 | 47 | 43 | 44 | F1 | 45 | 46 |
| nbMT78 | PDI | HC Rituximab | 6601 | 14 | 47 | 39 | 40 | F1 | 41 | 42 |
| atHSP69 | PDI | LC Rituximab | 6602 | 17 | 48 | 43 | 44 | F1 | 45 | 46 |
| atHSP69 | PDI | HC Rituximab | 6603 | 17 | 48 | 39 | 40 | F1 | 41 | 42 |
| nbEPI42 | PDI | LC Rituximab | 6604 | 19 | 49 | 43 | 44 | F1 | 45 | 46 |
| nbEPI42 | PDI | HC Rituximab | 6605 | 19 | 49 | 39 | 40 | F1 | 41 | 42 |
| nbSNS46 | PDI | LC Rituximab | 6606 | 21 | 50 | 43 | 44 | F1 | 45 | 46 |
| nbSNS46 | PDI | HC Rituximab | 6607 | 21 | 50 | 39 | 40 | F1 | 41 | 42 |
| nbCSY65 | PDI | LC Rituximab | 6608 | 23 | 51 | 43 | 44 | F1 | 45 | 46 |
| nbCSY65 | PDI | HC Rituximab | 6609 | 23 | 51 | 39 | 40 | F1 | 41 | 42 |
| nbHEL40 | PDI | LC Rituximab | 6610 | 25 | 52 | 43 | 44 | F1 | 45 | 46 |
| nbHEL40 | PDI | HC Rituximab | 6611 | 25 | 52 | 39 | 40 | F1 | 41 | 42 |
| nbSEP44 | PDI | LC Rituximab | 6612 | 27 | 53 | 43 | 44 | F1 | 45 | 46 |
| nbSEP44 | PDI | HC Rituximab | 6613 | 27 | 53 | 39 | 40 | F1 | 41 | 42 |
| CPMV 160 | PDI | H1 A-Mich-45-2015 | 3703 | 9 | — | 54 | — | 55 | 56 | 57 |
| nbMT78 | PDI | H1 A-Mich-45-2015 | 4703 | 14 | 47 | 54 | 55 | F1 | 56 | 57 |
| atHSP69 | PDI | H1 A-Mich-45-2015 | 4704 | 17 | 48 | 54 | 55 | F1 | 56 | 57 |
| nbEPI42 | PDI | H1 A-Mich-45-2015 | 6700 | 19 | 49 | 54 | 55 | F1 | 56 | 57 |
| nbSNS46 | PDI | H1 A-Mich-45-2015 | 6701 | 21 | 50 | 54 | 55 | F1 | 56 | 57 |
| nbCSY65 | PDI | H1 A-Mich-45-2015 | 6702 | 23 | 51 | 54 | 55 | F1 | 56 | 57 |
| nbHEL40 | PDI | H1 A-Mich-45-2015 | 6703 | 25 | 52 | 54 | 55 | F1 | 56 | 57 |
| nbSEP44 | PDI | H1 A-Mich-45-2015 | 6704 | 27 | 53 | 54 | 55 | F1 | 56 | 57 |
| CPMV 160 | PDI | H3 A-Sing-19-0019-16 | 4008 | 9 | — | 58 | — | 59 | 60 | 61 |
| nbMT78 | PDI | H3 A-Sing-19-0019-16 | 6134 | 14 | 47 | 58 | 59 | F1 | 60 | 61 |
| atHSP69 | PDI | H3 A-Sing-19-0019-16 | 6136 | 17 | 48 | 58 | 59 | F1 | 60 | 61 |
| nbEPI42 | PDI | H3 A-Sing-19-0019-16 | 6705 | 19 | 49 | 58 | 59 | F1 | 60 | 61 |
| nbSNS46 | PDI | H3 A-Sing-19-0019-16 | 6706 | 21 | 50 | 58 | 59 | F1 | 60 | 61 |
| nbCSY65 | PDI | H3 A-Sing-19-0019-16 | 6707 | 23 | 51 | 58 | 59 | F1 | 60 | 61 |
| nbHEL40 | PDI | H3 A-Sing-19-0019-16 | 6708 | 25 | 52 | 58 | 59 | F1 | 60 | 61 |
| nbSEP44 | PDI | H3 A-Sing-19-0019-16 | 6709 | 27 | 53 | 58 | 59 | F1 | 60 | 61 |

*SOI: sequence of interest;
Primer 1: Primer 1 (For In-fusion cloning);
Primer 2: Primer 2 (to create fragment no 1 to amplify GOI with primer 3):
Primer 3: Primer 3 (For In-fusion cloning)

### Example 2

### Methods

Agrobacterium tumefaciens Transfection

Agrobacterium tumefaciens strain AGL1 was transfected (transformed) by electroporation with the different expression vectors using the methods described by D'Aoust et al., 2008 (Plant Biotech. J. 6:930-40). Transfected Agrobacterium were grown in LB medium supplemented with 10 mM 2-(N-morpholino) ethanesulfonic acid (MES) and 50 μg/ml kanamycin pH5.6 to an OD₆₀₀ between 0.6 and 1.6 and frozen in 100 μl aliquots.

### Preparation of Plant Biomass, Inoculum and Agroinfiltration

N. benthamiana plants were grown from seeds in flats filled with a commercial peat moss substrate. The plants were allowed to grow in the greenhouse under a 16/8 photoperiod and a temperature regime of 25° C. day/20° C. night. Three weeks after seeding, individual plantlets were picked out, transplanted in pots and left to grow in the greenhouse for three additional weeks under the same environmental conditions

Agrobacteria transfected (transformed) with each expression vector were grown in a LB medium supplemented with 10 mM 2-(N-morpholino) ethanesulfonic acid (MES) and 50 μg/ml kanamycin pH5.6 until they reached an OD₆₀₀ between 0.6 and 1.6. Agrobacterium suspensions were centrifuged before use and resuspended in infiltration medium (10 mM MgCl₂ and 10 mM MES pH 5.6) and stored overnight at 4° C. On the day of infiltration, culture batches were diluted in 2.5 culture volumes and allowed to warm before use. Whole plants of N. benthamiana were placed upside down in the bacterial suspension in an air-tight stainless steel tank under a vacuum of 20-40 Torr for 2-min. Plants were returned to the greenhouse for a 6 or 9 day incubation period until harvest.

### Leaf Harvest and Total Protein Extraction

Following incubation, the aerial part of plants was harvested, frozen at −80° C. and crushed into pieces. Total soluble proteins were extracted by homogenizing (Polytron) each sample of frozen-crushed plant material in 2 volumes of cold 50 mM Tris buffer pH 8.0+500 mM NaCl, 0.4 μg/ml Metabisulfite and 1 mM phenylmethanesulfonyl fluoride. After homogenization, the slurries were centrifuged at 10,000 g for 10 min at 4° C. and these clarified crude extracts (supernatant) kept for analyses.

The total protein content of clarified crude extracts was determined by the Bradford assay (Bio-Rad, Hercules, California) using bovine serum albumin as the reference standard. Proteins were separated by SDS-PAGE under reducing conditions using Criterion™ TGX Stain-Free™ precast gels (Bio-Rad Laboratories, Hercules, CA). Proteins were visualized by staining the gels with Coomassie Brilliant Blue. Alternatively, proteins were visualized with Gel Doc™ EZ imaging system (Bio-Rad Laboratories, Hercules, CA) and electrotransferred onto polyvinylene difluoride (PVDF) membranes (Roche Diagnostics Corporation, Indianapolis, Indiana) for immunodetection. Prior to immunoblotting, the membranes were blocked with 5% skim milk and 0.1% Tween-20 in Tris-buffered saline (TBS-T) for 16-18 h at 4° C.

### Dasher Expression as Determined by Direct Fluorescence in Crude Extract

Dasher expression was quantified by direct measure of fluorescence in crude extracts. Frozen biomass was extracted using 50 mM Tris+150 mM NaCl pH 7.4 extraction buffer by mechanical extraction and centrifuged 10 minutes at 10000 g at 4° C. to remove insoluble debris. Clarified crude extracts were diluted 1/16, 1/48 and 1/144 in PBS and fluorescence was measured using a Fluoroskan (Ascent) instrument using 485 nm as excitation filter and 518 nm as emission filter.

### HA Expression as Determined Using Hemagglutination Assay (HA Titer)

Hemagglutination assay was based on a method described by Nayak and Reichl (2004, J. Virol. Methods 122:9-15). Serial double dilutions of the test samples (100 μL) were made in V-bottomed 96-well microtiter plates containing 100 μL PBS, leaving 100 μL of diluted sample per well. One hundred microliters of a 0.25% turkey red blood cells suspension (Bio Link Inc., Syracuse, NY; for all B strains, H1, H5 and H7) or 0.5% guinea pig red blood cells suspension (for H3) were added to each well, and plates were incubated for 2 h at room temperature. The reciprocal of the highest dilution showing complete hemagglutination was recorded as HA activity.

### Rituximab Expression as Determined by In-Gel Densitometry

For Rituximab expression analysis, crude protein extracts (2 g biomass/EU) were produced from leaves by mechanical extraction in 150 mM Tris, pH 7.4 buffer with 150 mM NaCl and the extracts were electrophoresed on SDS-PAGE under non-reducing conditions for in-gel densitometry quantification of the band corresponding to the fully assembled H₂L₂ form of the antibody. Protein electrophoresis was performed in Stain-Free gels from Bio-Rad and gel imaging system was carried-out using Gel Doc XR+ system, including the Image Lab software for image analyses and in-gel quantification.

### Analysis of VLP Formation/Iodixanol Gradients

Proteins were extracted from frozen biomass by mechanical extraction in a blender with 2 volumes of extraction buffer (100 mM phosphate buffer pH 7.2+150 mM NaCl). The slurry was filtered through a large pore nylon filter to remove large debris and centrifuged 5000 g for 5 min at 4° C. The supernatant was collected and centrifuged again at 5000 g for 30 min (4° C.) to remove additional debris. The supernatant is then loaded on a discontinuous iodixanol density gradient. Analytical density gradient centrifugation was performed as follows: 38 ml tubes containing discontinuous iodixanol density gradient in acetate buffer (1 ml at 45%, 2 ml at 35%, 2 ml at 33%, 2 ml at 31%, 2 ml at 29% and 5 ml at 25% of iodixanol) were prepared and overlaid with 25 ml of the extracts containing the virus-like particles. The gradients were centrifuged at 175 000 g for 4 hours (4° C.). After centrifugation, 1 ml fractions were collected from the bottom to the top and fractions were analyzed by SDS-PAGE combined with protein staining or Western blot.

### Example 3

### Protein Production in Plants

N. benthamiana leaves were, vacuum infiltrated, as described in Example 2, with Agrobacterium tumefaciens comprising expression vectors encoding the protein of interest operatively linked to the defined expression enhancer, to permit expression of the protein of interest, and the leaves examined for the protein of interest production. After 9 days post infiltration (DPI), total crude protein extracts were prepared from leaf homogenates, and hemagglutinin titer was determined as described above.

With reference to FIG. 2A, each of the expression enhancers, nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5), operatively linked to a nucleic acid sequence encoding Dasher was observed to result in the same or increased expression of the protein, when compared to the activity of the prior art expression enhancer sequence CMPV 160 (WO 2015/103704) operatively linked to the same nucleic acid sequence encoding the same proteins of interest, and expressed under similar conditions. The activity of these enhancer elements was the same or greater than the expression enhancers nbMT78 and atHSP69 (described in U.S. Provisional Application No. 62/643,053, filed Mar. 14, 2018, which is incorporated herein by reference).

With reference to FIGS. 3A and 3B, each of the expression enhancers, nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5), operatively linked to a nucleic acid sequence encoding a H3 A/Singapore, or a H1 Michigan/45/15, was observed to result in a similar, or slightly increased, expression of the protein when compared to the activity of the prior art expression enhancer sequence CMPV 160 (WO 2015/103704) operatively linked to the same nucleic acid sequence encoding the same proteins of interest, and expressed under similar conditions. The activity of these enhancer elements was also the same or greater than the expression enhancers nbMT78 and atHSP69 (described in U.S. Provisional Application No. 62/643,053, filed Mar. 14, 2018, which is incorporated herein by reference).

These results demonstrate the expression enhancer sequences described herein may be used for the expression of a protein of interest that is operatively linked to the expression enhancer, in a plant, portion of plant, or a plant cell.

### Example 4

### Norovirus VP1 Protein and VLP Production in Plants

N. benthamiana leaves were vacuum infiltrated, as described in Example 2, with Agrobacterium tumefaciens comprising expression vectors encoding norovirus VP1 from GII.4 genotype, and the leaves examined for VLP production. After 9 days post infiltration (DPI), total crude protein extracts were separated by SDS-PAGE, and stained with Coomassie (VP1 production), or separated using discontinuous iodixanol density gradients as described in Example 2, above (VLP production). Fractions from the density gradients were examined using Coomassie-stained SDS-PAGE. Norovirus VP1 proteins appear at an approximate 55-60 kDa band. The occurrence of the VP1 protein within a fraction of the density gradients is indicative of the fraction(s) to which the VLPs equilibrate during density gradient centrifugation. The yield of VLPs obtained from peak fractions after density gradient centrifugation was also determined.

As shown in FIG. 2B each of the expression enhancers, nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5), operatively linked to a nucleic acid sequence encoding norovirus GII.4/Syd/12 VP1 was observed to result in a similar, or slightly increased, expression of the protein when compared to the activity of the prior art expression enhancer sequence CMPV 160 (WO 2015/103704) operatively linked to the same nucleic acid sequence encoding the same proteins of interest, and expressed under similar conditions. The activity of these enhancer elements was also the same or greater than the expression enhancers nbMT78 and atHSP69 (described in U.S. Provisional Application No. 62/643,053, filed Mar. 14, 2018, which is incorporated herein by reference).

### Example 5

### Multimeric Protein Production in Plants

The expression enhancers described herein may also be used for the production of a multimeric protein of interest, for example an antibody. N. benthamiana leaves were, vacuum infiltrated, as described in Example 2, with Agrobacterium tumefaciens comprising expression vectors encoding the protein of interest operatively linked to the defined expression enhancer, to permit expression of the protein of interest, and the leaves examined for the protein of interest production. After 9 days post infiltration (DPI), total crude protein extracts were prepared from leaf homogenates were separated by SDS-PAGE and expression levels were determined by in-gel densitometry of complete IgG, as described above.

As shown with reference to FIG. 3C, co-expression of two nucleic acids, the first nucleic acid encoding the light chain (LC) of Rituximab operatively linked to one of the following expression enhancers, nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5), and the second nucleic acid encoding the heavy chain (HC) of rituximab operatively linked to one of the following expression enhancers, nbEPI42 (SEQ ID NO:1); nbSNS46 (SEQ ID NO:2); nbCSY65 (SEQ ID NO:3); nbHEL40 (SEQ ID NO:4); and nbSEP44 (SEQ ID NO:5), resulted in the same, or increased, yield of the multimeric protein in a plant, when compared to co-expression of first and second nucleic acids encoding the same HC and LC rituximab sequences, with each of the first and second nucleic acids operatively linked to the prior art expression enhancer CPMV 160. The activity of these enhancer elements was also the same or greater than the expression enhancers nbMT78 and atHSP69 (described in U.S. Provisional Application No. 62/643,053, filed Mar. 14, 2018, which is incorporated herein by reference).

These results demonstrate that the expression enhancers described herein may be used to produce multimeric proteins, such as an antibody, and that the same or different the expression enhancers as described herein may be operatively linked to each of the nucleic acid sequences used to encode the components of the multimeric protein.

All citations are hereby incorporated by reference.

The present invention has been described with regard to one or more embodiments. However, it will be apparent to persons skilled in the art that a number of variations and modifications can be made without departing from the scope of the invention as defined in the claims.

We claim:

1. A nucleic acid comprising an expression enhancer operatively linked with a heterologous nucleotide sequence encoding a protein of interest, the expression enhancer consisting of:
nbHEL40 (SEQ ID NO:4).

2. The nucleic acid of claim 1, where the heterologous nucleotide sequence encodes a viral protein or an antibody.

3. The nucleic acid of claim 2, wherein the viral protein is an influenza protein or a norovirus protein.

4. The nucleic acid of claim 3, wherein the influenza protein is a hemagglutinin protein selected from the group of H1, H2, H3, H4, H5, H6, H7, H8, H9, H10, H11, H12, H13, H14, H15, H16, and an influenza type B hemagglutinin.

5. The nucleic acid of claim 3, wherein the norovirus protein is a VP1, a VP2, or a combination thereof selected from the group of GI.1, GI.2, GI.3, GI.5, GI.7, GII.1, GII.2, GII.3, GII.4, GII.5, GII.6, GII.7, GII.12, GII.13, GII.14, GII.17 and GII.21.

6. A plant expression system comprising one or more than one of the nucleic acid of claim 1.

7. A plant expression system comprising one or more than one of nucleic acid of claim 2.

8. A plant expression system comprising one or more than one of the nucleic acid of claim 3.

9. A plant expression system comprising one or more than one of the nucleic acid of claim 4.

10. A plant expression system comprising one or more than one of the nucleic acid of claim 5.

11. The plant expression system of claim 6 further comprising a comovirus 3′ UTR.

12. A method of producing a protein of interest in a plant, a portion of a plant, or a plant cell, comprising,
introducing into the plant, the portion of a plant, or the plant cell, in a stable or transient manner, the plant expression system of claim 6 comprising the one or more than one nucleic acid, and
incubating the plant or the portion of a plant under conditions that permit expression of each of the heterologous nucleotide sequence encoding the protein of interest.

13. The method of claim 12, wherein the protein of interest is a viral protein.

14. The method of claim 13, wherein the viral protein is an influenza protein or a norovirus protein.

15. The method of claim 14, wherein the influenza protein is a hemagglutinin protein selected from the group of H1, H2, H3, H4, H5, H6, H7, H8, H9, H10, H11, H12, H13, H14, H15, H16, and an influenza type B hemagglutinin.

16. The method of claim 14, wherein the norovirus protein is a VP1, a VP2, or a combination thereof selected from the group of GI.1, GI.2, GI,3, GI.5, GI.7, GII.1, GII.2, GII.3, GII.4, GII.5, GII.6, GII.7, GII.12, GII.13, GII.14, GII.17 and GII.21

17. The method of claim 12, wherein the protein of interest is a multimeric protein, and the step of introducing involves co-expressing two or more than two of the heterologous nucleotide sequence, each of the two or more than two of the heterologous nucleotide sequence encoding a component of the multimeric protein.

18. A plant, portion of a plant, or a plant cell, transiently transformed or stably transformed with the plant expression system of claim 6.

19. The nucleic acid of claim 1, wherein the expression enhancer increases expression of the heterologous nucleotide sequence encoding the protein of interest in a Nicotiana benthamiana plant when compared to the level of expression of the same heterologous nucleotide sequence that is not operatively linked to the expression enhancer.


SEQUENCE LISTING (65 sequences)

SEQ ID NO: 1 - DNA, 42 bp, synthetic construct
  misc_feature 1..42: note = Expression enhancer nbEP142
  source 1..42: mol_type = other DNA
actttaatttgctgattttcaacaaaatcaagaatttcagca

SEQ ID NO: 2 - DNA, 46 bp, synthetic construct
  misc_feature 1..46: note = Expression enhancer nbSNS46
  source 1..46: mol_type = other DNA
attcagtgcttaactggttattgagtaagttatcaaaaagcaaaaa

SEQ ID NO: 3 - DNA, 65 bp, synthetic construct
  misc_feature 1..65: note = Expression enhancer nbCYS65
  source 1..65: mol_type = other DNA
acttttctaatcaatcatcaaacagaacgcagaaaatttcctaaaaacaaaaaaaaggcatacaa

SEQ ID NO: 4 - DNA, 40 bp, synthetic construct
  misc_feature 1..40: note = Expression enhancer nbHEL40
  source 1..40: mol_type = other DNA
actccatttgaatctatcaaaccaaaacacattgagcaaa

SEQ ID NO: 5 - DNA, 44 bp, synthetic construct
  misc_feature 1..44: note = Expression enhancer nbSEP44
  source 1..44: mol_type = other DNA
acttcaatcactccacactttattctctttcaaaacctacactc

SEQ ID NO: 6 - DNA, 160 bp, Cowpea Mosiac Virus
  source 1..160: mol_type = genomic DNA
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtaca

SEQ ID NO: 7 - DNA, 78 bp, synthetic construct
  misc_feature 1..78: note = Primer nbMT78
  source 1..78: mol_type = other DNA
acacaatttgctttagtgattaaactttcttttacaacaaattaaaggtctattatctcccaacaacataagaaaaca

SEQ ID NO: 8 - DNA, 69 bp, synthetic construct
  misc_feature 1..69: note = Primer atHSP69
  source 1..69: mol_type = other DNA
aaattcaaaatttaacacacaaacacaaacacacacaccaaaaaaaacacagaccttaaaaaaataaaa

SEQ ID NO: 9 - DNA, 48 bp, synthetic construct
  misc_feature 1..48: note = Primer IF-(2X35S+C)_CPMV160.c
  source 1..48: mol_type = other DNA
tttcatttggagaggctattaaaatcttaataggttttgataaaagcg

SEQ ID NO: 10 - DNA, 56 bp, synthetic construct
  misc_feature 1..56: note = Primer IF-Dasher(27-609).r
  source 1..56: mol_type = other DNA
actaaagaaaataggcctttactgataggtatcgagatcgacggccttgaccactt

SEQ ID NO: 11 - DNA, 871 bp, synthetic construct
  misc_feature 1..871: note = CPMV 160 5'UTR-Dasher DNA sequence
  source 1..871: mol_type = other DNA
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatgactgccctgaccgaaggtgctaagctgtttgagaaggagattccgtacatcaccgagctggaaggggacgtcgaaggaatgaagttcatcatcaagggagaaggaaccggggacgctacgactggaaccattaaggccaagtatatctgtaccactggagatctgccagtgccttgggccacccttgtgtcaaccctctcgtatggagtgcagtgttttgctaagtaccctagccacattaaggacttcttcaaatccgccatgccggaaggttatacccaagagcgcaccatttcttttgagggagatggagtgtacaagacccgcgcgatggtcacctatgagaggggatctatctacaaccgggtgactctgactggagaaaactttaagaaggacgggcatattcttcggaagaatgtcgccttccagtgccctcccagcatcctttacattctccccgacactgtgaacaacggaatccgcgtggagttcaatcaagcctacgacatcgagggggtgacggagaagctggtgaccaagtgtagccagatgaatcggccactggccggttcagcggctgtccacattccgcgctaccatcatatcacttatcacactaagctctccaaagaccgcgatgagaggagagatcacatgtgcctggtggaagtggtcaaggccgtcgatctcgatacctatcagtaa

SEQ ID NO: 12 - DNA, 711 bp, synthetic construct
  misc_feature 1..711: note = Dasher DNA sequence
  source 1..711: mol_type = other DNA
atgactgccctgaccgaaggtgctaagctgtttgagaaggagattccgtacatcaccgagctggaaggggacgtcgaaggaatgaagttcatcatcaagggagaaggaaccggggacgctacgactggaaccattaaggccaagtatatctgtaccactggagatctgccagtgccttgggccacccttgtgtcaaccctctcgtatggagtgcagtgttttgctaagtaccctagccacattaaggacttcttcaaatccgccatgccggaaggttatacccaagagcgcaccatttcttttgagggagatggagtgtacaagacccgcgcgatggtcacctatgagaggggatctatctacaaccgggtgactctgactggagaaaactttaagaaggacgggcatattcttcggaagaatgtcgccttccagtgccctcccagcatcctttacattctccccgacactgtgaacaacggaatccgcgtggagttcaatcaagcctacgacatcgagggggtgacggagaagctggtgaccaagtgtagccagatgaatcggccactggccggttcagcggctgtccacattccgcgctaccatcatatcacttatcacactaagctctccaaagaccgcgatgagaggagagatcacatgtgcctggtggaagtggtcaaggccgtcgatctcgatacctatcagtaa

SEQ ID NO: 13 - protein, 236 aa, synthetic construct
  REGION 1..236: note = Dasher protein sequence
MTALTEGAKLFEKEIPYITELEGDVEGMKFIIKGEGTGDATTGTIKAKYICTTGDLPVPWATLVSTLSYGVQCFAKYPSHIKDFFKSAMPEGYTQERTISFEGDGVYKTRAMVTYERGSIYNRVTLTGENFKKDGHILRKNVAFQCPPSILYILPDTVNNGIRVEFNQAYDIEGVTEKLVTKCSQMNRPLAGSAAVHIPRYHHITYHTKLSKDRDERRDHMCLVEVVKAVDLDTYQ

SEQ ID NO: 14 - DNA, 89 bp, synthetic construct
  misc_feature 1..89: note = Primer IF-nbMT78.c
  source 1..89: mol_type = other DNA
tttcatttggagaggcacacaatttgctttagtgattaaactttcttttacaacaaattaaaggtctattatctcccaacaacataaga

SEQ ID NO: 15 - DNA, 59 bp, synthetic construct
  misc_feature 1..59: note = Primer nbMT78_Dasher.c
  source 1..59: mol_type = other DNA
ttaaaggtctattatctcccaacaacataagaaaacaatgactgccctgaccgaaggtg

SEQ ID NO: 16 - DNA, 59 bp, synthetic construct
  misc_feature 1..59: note = Primer atHSP69_Dasher.c
  source 1..59: mol_type = other DNA
ccaaaaaaaacacagaccttaaaaaaataaaaatgactgccctgaccgaaggtgctaag

SEQ ID NO: 17 - DNA, 84 bp, synthetic construct
  misc_feature 1..84: note = Primer IF-atHSP69.c
  source 1..84: mol_type = other DNA
tttcatttggagaggcaaattcaaaatttaacacacaaacacaaacacacacaccaaaaaaaacacagaccttaaaaaaataaa

SEQ ID NO: 18 - DNA, 52 bp, synthetic construct
  misc_feature 1..52: note = Primer nbEPI42+Dasher.c
  source 1..52: mol_type = other DNA
tttcaacaaaatcaagaatttcagcaatgactgccctgaccgaaggtgctaa

SEQ ID NO: 19 - DNA, 59 bp, synthetic construct
  misc_feature 1..59: note = Primer IF-nbEPI42.c
  source 1..59: mol_type = other DNA
atttcatttggagaggcactttaatttgctgattttcaacaaaatcaagaatttcagca

SEQ ID NO: 20 - DNA, 52 bp, synthetic construct
  misc_feature 1..52: note = Primer nbSNS46+Dasher.c
  source 1..52: mol_type = other DNA
ttgagtaagttatcaaaaagcaaaaaatgactgccctgaccgaaggtgctaa

SEQ ID NO: 21 - DNA, 63 bp, synthetic construct
  misc_feature 1..63: note = Primer IF-nbSNS46.c
  source 1..63: mol_type = other DNA
atttcatttggagaggcattcagtgcttaactggttattgagtaagttatcaaaaagcaaaaa

SEQ ID NO: 22 - DNA, 52 bp, synthetic construct
  misc_feature 1..52: note = Primer nbCSY65+Dasher.c
  source 1..52: mol_type = other DNA
cctaaaaacaaaaaaaaggcatacaaatgactgccctgaccgaaggtgctaa

SEQ ID NO: 23 - DNA, 82 bp, synthetic construct
  misc_feature 1..82: note = Primer IF-nbCSY65.c
  source 1..82: mol_type = other DNA
atttcatttggagaggcacttttctaatcaatcatcaaacagaacgcagaaaatttcctaaaaacaaaaaaaaggcatacaa

SEQ ID NO: 24 - DNA, 53 bp, synthetic construct
  misc_feature 1..53: note = Primer nbHEL40+Dasher.c
  source 1..53: mol_type = other DNA
ctatcaaaccaaaacacattgagcaaaatgactgccctgaccgaaggtgctaa

SEQ ID NO: 25 - DNA, 56 bp, synthetic construct
  misc_feature 1..56: note = Primer IF-nbHEL40.c
  source 1..56: mol_type = other DNA
atttcatttggagaggcactccatttgaatctatcaaaccaaaacacattgagcaa

SEQ ID NO: 26 - DNA, 53 bp, synthetic construct
  misc_feature 1..53: note = Primer nbSEP44+Dasher.c
  source 1..53: mol_type = other DNA
tttattctctttcaaaacctacactcatgactgccctgaccgaaggtgctaag

SEQ ID NO: 27 - DNA, 61 bp, synthetic construct
  misc_feature 1..61: note = Primer IF-nbSEP44.c
  source 1..61: mol_type = other DNA
atttcatttggagaggcacttcaatcactccacactttattctctttcaaaacctacactc

SEQ ID NO: 28 - DNA, 48 bp, synthetic construct
  misc_feature 1..48: note = Primer IF-GII4Syd12VP1.r
  source 1..48: mol_type = other DNA
actaaagaaaataggccttcagacagccctgcgtctgccagtcccatt

SEQ ID NO: 29 - DNA, 1783 bp, synthetic construct
  misc_feature 1..1783: note = CPMV 160 5'UTR-VP1 (GII.4) DNA sequence
  source 1..1783: mol_type = other DNA
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatgaaaatggcctcgagtgacgctaaccctagtgacggcagcgccgccaatcttgtgcctgaggttaataatgaggtgatggccctggagcctgtggtgggcgcagccatagcagcgcccgtggccggtcagcagaatgtgattgacccgtggatacgcaacaattttgtccaagcccctggtggggagttcaccgttagcccgagaaatgcgccaggagaaatcctgtggtcggccagcttgggacccgatctgaacccctatttgtcacatctcgctcggatgtacaacgggtatgccggcggatttgaagtgcaggtgattctggctgggaacgcgttcactgctggcaaagtgatctttgcagcggtgcctcccaacttccccactgaaggactgtctccaagccaggtcacaatgtttccacacatcgtggtggacgtacggcagctagagcctgtcctgattcccctccctgatgtacgcaataatttctaccactacaatcaatccaatgatccgaccattaaactcatcgcgatgttgtacacccctctgcgcgctaacaatgctggagacgacgtattcaccgtgtcatgcagagtgctcaccagaccttcaccagactttgactttatcttcttagtgccccccactgttgagagccgaaccaagccctttagtgtccccgtactcacagtcgaggagatgacaaatagccgctttccaatcccccttgagaaactgttcacaggaccttcctcggcattcgtggttcagccacagaacggacgctgcacaactgacggcgtgctgctcggaaccacccagcttagccctgttaatatctgtacgtttagaggcgacgtaactcacataactggctcacggaactataccatgaatctggcatcacagaattggaatgactacgacccaaccgaagagattcccgcacctcttggaacccccgactttgtgggaaaaatacagggcgtcctgacacaaaccaccagaaccgatggctccacacggggacacaaggcaaccgtctacactggctctgccgattttgccccgaaactgggtagagtgcagtttgagaccgacactgaccgggactttgaagccaatcagaatactaagttcacacctgtaggagtgattcaggacgggggcaccactcaccggaacgagccgcaacaatgggtcctgccctcttatagcgggaggaatactcataatgtgcatttggctcctgcagtggctcccacgtttcccggggaacaactgctcttttttcgttcaaccatgcctggatgctccggatatcccaatatggatctcgattgcctgctcccacaggaatgggtgcagtatttttatcaagaggccgcaccagcccaatccgacgtcgcacttctgcggttcgtgaatccagacacaggccgcgtgttgtttgagtgcaaattgcacaaatcaggatacgttacagtggctcatactggacagcatgacctggtgatcccacccaacggatattttaggttcgactcctgggtgaatcagttttatacattagcccccatggggaatgggactggcagacgcagggctgtctga

SEQ ID NO: 30 - DNA, 1623 bp, synthetic construct
  misc_feature 1..1623: note = VP1 (GII.4) DNA sequence
  source 1..1623: mol_type = other DNA
atgaaaatggcctcgagtgacgctaaccctagtgacggcagcgccgccaatcttgtgcctgaggttaataatgaggtgatggccctggagcctgtggtgggcgcagccatagcagcgcccgtggccggtcagcagaatgtgattgacccgtggatacgcaacaattttgtccaagcccctggtggggagttcaccgttagcccgagaaatgcgccaggagaaatcctgtggtcggccagcttgggacccgatctgaacccctatttgtcacatctcgctcggatgtacaacgggtatgccggcggatttgaagtgcaggtgattctggctgggaacgcgttcactgctggcaaagtgatctttgcagcggtgcctcccaacttccccactgaaggactgtctccaagccaggtcacaatgtttccacacatcgtggtggacgtacggcagctagagcctgtcctgattcccctccctgatgtacgcaataatttctaccactacaatcaatccaatgatccgaccattaaactcatcgcgatgttgtacacccctctgcgcgctaacaatgctggagacgacgtattcaccgtgtcatgcagagtgctcaccagaccttcaccagactttgactttatcttcttagtgccccccactgttgagagccgaaccaagccctttagtgtccccgtactcacagtcgaggagatgacaaatagccgctttccaatcccccttgagaaactgttcacaggaccttcctcggcattcgtggttcagccacagaacggacgctgcacaactgacggcgtgctgctcggaaccacccagcttagccctgttaatatctgtacgtttagaggcgacgtaactcacataactggctcacggaactataccatgaatctggcatcacagaattggaatgactacgacccaaccgaagagattcccgcacctcttggaacccccgactttgtgggaaaaatacagggcgtcctgacacaaaccaccagaaccgatggctccacacggggacacaaggcaaccgtctacactggctctgccgattttgccccgaaactgggtagagtgcagtttgagaccgacactgaccgggactttgaagccaatcagaatactaagttcacacctgtaggagtgattcaggacgggggcaccactcaccggaacgagccgcaacaatgggtcctgccctcttatagcgggaggaatactcataatgtgcatttggctcctgcagtggctcccacgtttcccggggaacaactgctcttttttcgttcaaccatgcctggatgctccggatatcccaatatggatctcgattgcctgctcccacaggaatgggtgcagtatttttatcaagaggccgcaccagcccaatccgacgtcgcacttctgcggttcgtgaatccagacacaggccgcgtgttgtttgagtgcaaattgcacaaatcaggatacgttacagtggctcatactggacagcatgacctggtgatcccacccaacggatattttaggttcgactcctgggtgaatcagttttatacattagcccccatggggaatgggactggcagacgcagggctgtctga

SEQ ID NO: 31 - protein, 540 aa, synthetic construct
  REGION 1..540: note = VP1 (GII.4) protein sequence
MKMASSDANPSDGSAANLVPEVNNEVMALEPVVGAAIAAPVAGQQNVIDPWIRNNFVQAPGGEFTVSPRNAPGEILWSASLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNDYDPTEEIPAPLGTPDFVGKIQGVLTQTTRTDGSTRGHKATVYTGSADFAPKLGRVQFETDTDRDFEANQNTKFTPVGVIQDGGTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 32 - DNA, 59 bp, synthetic construct
  misc_feature 1..59: note = Primer nbMT78+GII4Syd12.c
  source 1..59: mol_type = other DNA
tctattatctcccaacaacataagaaaacaatgaaaatggcctcgagtgacgctaaccc

SEQ ID NO: 33 - DNA, 59 bp, synthetic construct
  misc_feature 1..59: note = Primer atHSP69+GII4Syd12.c
  source 1..59: mol_type = other DNA
aaacacagaccttaaaaaaataaaaatgaaaatggcctcgagtgacgctaaccctagtg

SEQ ID NO: 34 - DNA, 56 bp, synthetic construct
  misc_feature 1..56: note = Primer nbEPI42+GII4Syd12.c
  source 1..56: mol_type = other DNA
tgattttcaacaaaatcaagaatttcagcaatgaaaatggcctcgagtgacgctaa

SEQ ID NO: 35 - DNA, 52 bp, synthetic construct
  misc_feature 1..52: note = nbSNS46+GII4Syd12.c
  source 1..52: mol_type = other DNA
ttgagtaagttatcaaaaagcaaaaaatgaaaatggcctcgagtgacgctaa

SEQ ID NO: 36 - DNA, 53 bp, synthetic construct
  misc_feature 1..53: note = Primer nbCSY65+GII4Syd12.c
  source 1..53: mol_type = other DNA
tcctaaaaacaaaaaaaaggcatacaaatgaaaatggcctcgagtgacgctaa

SEQ ID NO: 37 - DNA, 56 bp, synthetic construct
  misc_feature 1..56: note = Primer nbHEL40+GII4Syd12.c
  source 1..56: mol_type = other DNA
tctatcaaaccaaaacacattgagcaaaatgaaaatggcctcgagtgacgctaacc

SEQ ID NO: 38 - DNA, 53 bp, synthetic construct
  misc_feature 1..53: note = Primer nbSEP44+GII4Syd12.c
  source 1..53: mol_type = other DNA
ctttattctctttcaaaacctacactcatgaaaatggcctcgagtgacgctaa

SEQ ID NO: 39 - DNA, 51 bp, synthetic construct
  misc_feature 1..51: note = Primer IF -HC(Ritux).s1-6r
  source 1..51: mol_type = other DNA
actaaagaaaataggccttcactttccaggagaaagagaaagggacttttg

SEQ ID NO: 40 - DNA, 1588 bp, synthetic construct
  misc_feature 1..1588: note = CPMV 160 5'UTR-PDI+Rituximab HC DNA sequence
  source 1..1588: mol_type = other DNA
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgcaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagcctagggaaccacaagtgtacactcttccaccatctagggatgagcttactaagaaccaagtttctcttacttgtcttgtgaagggattttatccatctgacatcgccgtggaatgggaatccaacggacaaccagagaacaattacaagactactccaccagttcttgattctgatggatccttctttctttattccaagcttactgttgataagtccagatggcagcaaggaaatgtgttctcttgttctgttatgcacgaagctcttcataatcattatactcaaaagtccctttctctttctcctggaaagtga

SEQ ID NO: 41 - DNA, 1428 bp, synthetic construct
  misc_feature 1..1428: note = PDI+Rituximab HC DNA sequence
  source 1..1428: mol_type = other DNA
atggcgaaaaacgttgcgattttcggcttattgttttctcttcttgtgttggttccttctcagatcttcgcgcaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagcctagggaaccacaagtgtacactcttccaccatctagggatgagcttactaagaaccaagtttctcttacttgtcttgtgaagggattttatccatctgacatcgccgtggaatgggaatccaacggacaaccagagaacaattacaagactactccaccagttcttgattctgatggatccttctttctttattccaagcttactgttgataagtccagatggcagcaaggaaatgtgttctcttgttctgttatgcacgaagctcttcataatcattatactcaaaagtccctttctctttctcctggaaagtga

SEQ ID NO: 42 - protein, 475 aa, synthetic construct
  REGION 1..475: note = PDI+Rituximab HC protein sequence
MAKNVAIFGLLFSLLVLVPSQIFAQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 51 bp, synthetic construct
  misc_feature 1..51: note = IF -LC(Ritux).s1-6r
  source 1..51: mol_type = other DNA
actaaagaaaataggccttcaacactctcccctgttgaagctctttgtgac

SEQ ID NO: 44 - DNA, 874 bp, synthetic construct
  misc_feature 1..874: note = CPMV 160 5'UTR-PDI+Rituximab LC DNA sequence
  source 1..874: mol_type = other DNA
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgcaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcacaatcagcagagtggaggctgaagatgctgccacttattactgccagcagtggactagtaacccacccacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 45 - DNA, 714 bp, synthetic construct
  misc_feature 1..714: note = PDI+Rituximab LC DNA sequence
  source 1..714: mol_type = other DNA
atggcgaaaaacgttgcgattttcggcttattgttttctcttcttgtgttggttccttctcagatcttcgcgcaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcacaatcagcagagtggaggctgaagatgctgccacttattactgccagcagtggactagtaacccacccacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 46 - protein, 237 aa, synthetic construct
  REGION 1..237: note = PDI+Rituximab LC protein sequence
MAKNVAIFGLLFSLLVLVPSQIFAQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - DNA, 59 bp, synthetic construct
  misc_feature 1..59: note = nbMT78_SpPDI.c
  source 1..59: mol_type = other DNA
tattatctcccaacaacataagaaaacaatggcgaaaaacgttgcgattttcggcttat

SEQ ID NO: 48 - DNA, 59 bp, synthetic construct
  misc_feature 1..59: note = atHSP69_SpPDI.c
  source 1..59: mol_type = other DNA
caaaaaaaacacagaccttaaaaaaataaaaatggcgaaaaacgttgcgattttcggct

SEQ ID NO: 49 - DNA, 54 bp, synthetic construct
  misc_feature 1..54: note = nbEPI42+PDI.c
  source 1..54: mol_type = other DNA
tttcaacaaaatcaagaatttcagcaatggcgaaaaacgttgcgattttcggct

SEQ ID NO: 50 - DNA, 53 bp, synthetic construct
  misc_feature 1..53: note = nbSNS46+PDI.c
  source 1..53: mol_type = other DNA
ttgagtaagttatcaaaaagcaaaaaatggcgaaaaacgttgcgattttcggc

SEQ ID NO: 51 - DNA, 53 bp, synthetic construct
  misc_feature 1..53: note = nbCSY65 plus PDI.c
  source 1..53: mol_type = other DNA
cctaaaaacaaaaaaaaggcatacaaatggcgaaaaacgttgcgattttcggc

SEQ ID NO: 52 - DNA, 55 bp, synthetic construct
  misc_feature 1..55: note = nbHEL40+PDI.c
  source 1..55: mol_type = other DNA
ctatcaaaccaaaacacattgagcaaaatggcgaaaaacgttgcgattttcggct

SEQ ID NO: 53 - DNA, 57 bp, synthetic construct
  misc_feature 1..57: note = nbSEP44+PDI.c
  source 1..57: mol_type = other DNA
tttattctctttcaaaacctacactcatggcgaaaaacgttgcgattttcggcttat

SEQ ID NO: 54 - DNA, 46 bp, synthetic construct
  misc_feature 1..46: note = IF-H1cTMCT.s1-4r
  source 1..46: mol_type = other DNA
actaaagaaaataggcctttaaatacatattctacactgtagagac

SEQ ID NO: 55 - DNA, 1882 bp, synthetic construct
  misc_feature 1..1882: note = CPMV 160 5'UTR-PDI+H1 Mich DNA sequence
  source 1..1882: mol_type = other DNA
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcggacacattatgtataggttatcatgcgaacaattcaacagacactgtagacacagtactagaaaagaatgtaacagtaacacactctgttaaccttctggaagacaagcataacggaaaactatgcaaactaagaggggtagccccattgcatttgggtaaatgtaacattgctggctggatcctgggaaatccagagtgtgaatcactctccacagcaagttcatggtcctacattgtggaaacatctaattcagacaatggaacgtgttacccaggagatttcatcaattatgaggagctaagagagcaattgagctcagtgtcatcatttgaaaggtttgagatattccccaagacaagttcatggcccaatcatgactcgaacaaaggtgtaacggcagcatgtcctcacgctggagcaaaaagcttctacaaaaacttgatatggctagttaaaaaaggaaattcatacccaaagcttaaccaatcctacattaatgataaagggaaagaagtcctcgtgctgtggggcattcaccatccatctactactgctgaccaacaaagtctctatcagaatgcagatgcatatgtttttgtggggacatcaagatacagcaagaagttcaagccggaaatagcaacaagacccaaagtgagggatcaagaagggagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacacccgagggtgctataaacaccagcctcccatttcagaatatacatccgatcacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacaggacacagcagtgggtaaagagttcaaccacctggaaaaaagaatagagaatctaaataaaaaagttgatgatggtttcctggacatttggacttacaatgccgaactgttggttctaatggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaa

SEQ ID NO: 56 - DNA, 1722 bp, synthetic construct
  misc_feature 1..1722: note = PDI+H1 Mich DNA sequence
  source 1..1722: mol_type = other DNA
atggcgaaaaacgttgcgattttcggcttattgttttctcttcttgtgttggttccttctcagatcttcgcggacacattatgtataggttatcatgcgaacaattcaacagacactgtagacacagtactagaaaagaatgtaacagtaacacactctgttaaccttctggaagacaagcataacggaaaactatgcaaactaagaggggtagccccattgcatttgggtaaatgtaacattgctggctggatcctgggaaatccagagtgtgaatcactctccacagcaagttcatggtcctacattgtggaaacatctaattcagacaatggaacgtgttacccaggagatttcatcaattatgaggagctaagagagcaattgagctcagtgtcatcatttgaaaggtttgagatattccccaagacaagttcatggcccaatcatgactcgaacaaaggtgtaacggcagcatgtcctcacgctggagcaaaaagcttctacaaaaacttgatatggctagttaaaaaaggaaattcatacccaaagcttaaccaatcctacattaatgataaagggaaagaagtcctcgtgctgtggggcattcaccatccatctactactgctgaccaacaaagtctctatcagaatgcagatgcatatgtttttgtggggacatcaagatacagcaagaagttcaagccggaaatagcaacaagacccaaagtgagggatcaagaagggagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacacccgagggtgctataaacaccagcctcccatttcagaatatacatccgatcacaattggaaaatgtccaaagtatgtaaaaagcacaaaattgagactggccacaggattgaggaatgttccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaagattactaacaaagtaaattctgttattgaaaagatgaatacacaggacacagcagtgggtaaagagttcaaccacctggaaaaaagaatagagaatctaaataaaaaagttgatgatggtttcctggacatttggacttacaatgccgaactgttggttctaatggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaaaaaatagatggggtaaagctggaatcaacaaggatttaccagattttggcgatctattcaactgtcgccagttcattggtactggtagtctccctgggggcaatcagcttctggatgtgctctaatgggtctctacagtgtagaatatgtatttaa

SEQ ID NO: 57 - protein, 573 aa, synthetic construct
  REGION 1..573: note = PDI plus H1 Mich protein sequence
MAKNVAIFGLLFSLLVLVPSQIFADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQDTAVGKEFNHLEKRIENLNKKVDDGFLDIWTYNAELLVLMENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 58 - DNA, 55 bp, synthetic construct
  misc_feature 1..55: note = IF-H3Minn15.r
  source 1..55: mol_type = other DNA
actaaagaaaataggccttcaaatgcaaatgttgcatctaatgttgcccttttgg

SEQ ID NO: 59 - DNA, 1885 bp, synthetic construct
  misc_feature 1..1885: note = CPMV 160 5'UTR-PDI+H3 sing DNA sequence
  source 1..1885: mol_type = other DNA
tattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaatggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaaaaatgaaagcttcaattggactggagtcactcaaaacggaacaagttctgcttgcataaggggctctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctggctcgggtgatcgggaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattagatgcaacatttgcatttga

SEQ ID NO: 60 - DNA, 1725 bp, synthetic construct
  misc_feature 1..1725: note = DNA
  source 1..1725: mol_type = other DNA
atggcgaaaaacgttgcgattttcggcttattgttttctcttcttgtgttggttccttctcagatcttcgcgcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtgaaatatgcgacagtcctcatcagatccttgatggagagaactgcacactaatagatgctctattgggagaccctcagtgtgatggctttcaaaataagaaatgggacctttttgttgaacgaagcaaagcctacagcaactgttacccttatgatgtgccggattatgcctcccttaggtcactagttgcctcatccggcacactggagtttaaaaatgaaagcttcaattggactggagtcactcaaaacggaacaagttctgcttgcataaggggctctagtagtagtttctttagtagattaaattggttgacccacttaaactacacatatccagcattgaacgtgactatgccaaacaaggaacaatttgacaaattgtacatttggggggttcaccacccgggtacggacaaggaccaaatcttcctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtaaacaggatcacatacggggcctgtcccagatatgttaagcatagcactctgaaattggcaacaggaatgcgaaatgtaccagagaaacaaactagaggcatatttggcgcaatagcgggtttcatagaaaatggttgggagggaatggtggatggttggtacggtttcaggcatcaaaattctgagggaagaggacaagcagcagatctcaaaagcactcaagcagcaatcgatcaaatcaatgggaagctggctcgggtgatcgggaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaatgaaacttatgaccacaatgtgtacagggatgaagcattaaacaaccggttccagatcaagggagttgagctgaagtcagggtacaaagattggatcctatggatttcctttgccatatcatcccttgtactgttagttgctttgttggggttcatcatgtgggcctgccaaaagggcaacattagatgcaacatttgcatttga

SEQ ID NO: 61 - protein, 574 aa, synthetic construct
  REGION 1..574: note = PDI+H3 sing protein sequence
MAKNVAIFGLLFSLLVLVPSQIFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLARVIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISSLVLLVALLGFIMWACQKGNIRCNICI

SEQ ID NO: 62 - DNA, 4337 bp, synthetic construct
  misc_feature 1..4337: note = Cloning vector 1666 from left to right T-DNA
  source 1..4337: mol_type = other DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcactcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagacgtccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 63 - DNA, 1959 bp, synthetic construct
  misc_feature 1..1959: note = Construct 4467 from 2X35S promoter to NOS terminator
  source 1..1959: mol_type = other DNA
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggcacacaatttgctttagtgattaaactttcttttacaacaaattaaaggtctattatctcccaacaacataagaaaacaatgactgccctgaccgaaggtgctaagctgtttgagaaggagattccgtacatcaccgagctggaaggggacgtcgaaggaatgaagttcatcatcaagggagaaggaaccggggacgctacgactggaaccattaaggccaagtatatctgtaccactggagatctgccagtgccttgggccacccttgtgtcaaccctctcgtatggagtgcagtgttttgctaagtaccctagccacattaaggacttcttcaaatccgccatgccggaaggttatacccaagagcgcaccatttcttttgagggagatggagtgtacaagacccgcgcgatggtcacctatgagaggggatctatctacaaccgggtgactctgactggagaaaactttaagaaggacgggcatattcttcggaagaatgtcgccttccagtgccctcccagcatcctttacattctccccgacactgtgaacaacggaatccgcgtggagttcaatcaagcctacgacatcgagggggtgacggagaagctggtgaccaagtgtagccagatgaatcggccactggccggttcagcggctgtccacattccgcgctaccatcatatcacttatcacactaagctctccaaagaccgcgatgagaggagagatcacatgtgcctggtggaagtggtcaaggccgtcgatctcgatacctatcagtaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 64 - DNA, 6156 bp, synthetic construct
  misc_feature 1..6156: note = Cloning vector 4160 from left to right T-DNA
  source 1..6156: mol_type = other DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacacgcgtggcgcgccctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatgagtcttctaaccgaggtcgaaacgcctatcagaaacgaatgggggtgcagatgcaacgattcaagtgatcctcttgttgttgccgcaagtataattgggattgtgcacctgatattgtggattattgatcgccttttttccaaaagcatttatcgtatctttaaacacggtttaaaaagagggccttctacggaaggagtaccagagtctatgagggaagaatatcgagaggaacagcagaatgctgtggatgctgacgatggtcattttgtcagcatagagctggagtaagagctctaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggaactaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagacgtcactcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagacgtccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 65 - DNA, 5502 bp, synthetic construct
  misc_feature 1..5502: note = Cloning vector 4170 from left to right T-DNA
  source 1..5502: mol_type = other DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagacgtcactcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagacgtccagattttggcgatctattcaactgtcgccagttcattggtactggtagtctccctgggggcaatcagtttctggatgtgctctaatgggtctctacagtgtagaatatgtatttaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctaggtaaaaatcccaattatatttggtctaatttagtttggtattgagtaaaacaaattcgaaccaaaccaaaatataaatatatagtttttatatatatgcctttaagactttttatagaattttctttaaaaaatatcaagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaatctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaaagaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaattctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta